(19) United States
(12) Patent Application Publication
(10) Pub. No.: US 2024/0076361 A1
(43) Pub. Date: Mar. 7, 2024

(54) ANTI-PT217 TAU ANTIBODY

(71) Applicant: EISAI R&D MANAGEMENT CO., LTD., Tokyo (JP)

(72) Inventors: Sonia CIEZAREK, Hatfield Herts (GB); Malcolm Ian ROBERTS, Hatfield Herts (GB); James Martin STADDON, Hatfield Herts (GB); Takashi OBARA, Tsukuba-shi, Ibaraki (JP); Yohei MUKAI, Kobe-shi, Hyogo (JP); Lal Kishan AGARWALA, Tsukuba-shi, Ibaraki (JP)

(21) Appl. No.: 18/273,355

(22) PCT Filed: Feb. 17, 2022
(86) PCT No.: PCT/JP2022/006458

§ 371 Date: Jul. 20, 2023

Related U.S. Application Data
(60) Provisional application No. 63/151,254, filed on Feb. 19, 2021.

(51) Int. Cl. C07K 16/18; G01N 33/68

(52) U.S. Cl. CPC C07K 16/18; G01N 33/6896; C07K 2317/34; C07K 2317/92; G01N 2333/4709; G01N 2800/2821

(57) ABSTRACT

Disclosed herein an anti-pT217 Tau antibody or a pT217 Tau-binding fragment thereof.

### TECHNICAL FIELD

The present invention generally relates to an anti-pT217 Tau antibody or a pT217 Tau-binding fragment thereof.

### BACKGROUND ART

Alzheimer's disease (AD) is the most common neurodegenerative disorder affecting 1 in 9 elderly individuals and accounting for dementia in more than 30 million people worldwide. Currently, there is no cure or therapeutic approach to prevent or manage this devastating disease. Histologically, AD is characterized in postmortem brain by the presence of extracellular plaques containing amyloid beta peptide (Aβ) and intracellular neurofibrillary tangles that consist of aggregated Tau protein. The precise relationship between Aβ and Tau is not well understood but it is well established that pathological Tau burden correlates with disease severity. It has been proposed that prevention or slowing the progression of Tau pathology is a promising strategy to therapcutically intervene in the disease and provide a significant benefit to patients and their caregivers. In elderly populations, mild cognitive impairment is loss of cognitive ability greater than expected for that stage of life (Gauthier et al., “Mild cognitive impairment”, Lancet, 367(9518): 1262-1270). It may persist at the same level of impairment, reverse even or, adding to the burden on sufferer, carers and others, be a precursor of dementia, including Alzheimer's disease. Limiting conversion of mild cognitive decline to Alzheimer's disease or other dementias is an important clinical aim. Identifying mild cognitive impairment sufferers at risk of such conversion requires diagnostic methods.

To enable new and effective medicines for AD to be tested in the clinic it is also important to unambiguously diagnose the disease with precision. Imaging methods such as positron emission tomography (PET) and biomarker measurements from cerebrospinal fluid (CSF) are routinely used in the clinic but a less invasive and low-cost diagnostic for AD would be extremely useful. Of particular importance is ability to diagnose the disease and follow progression when symptoms are mild or even at the pre-symptomatic stage. Recently, a form of the Tau protein phosphorylated on threonine (T) residue at amino acid position 217 (pT217 Tau) was proven to be superior to a currently used fluid-based biomarker (pT181 Tau) in cerebrospinal fluid (CSF) to report on disease severity (Barthelemy et al., “Cerebrospinal fluid phosphotau T217 outperforms T181 as a biomarker for the differential diagnosis of Alzheimer's disease and PET amyloid-positive patient identification”, Alzheimer's Res. Ther., 2020 Vol. 12:26). In a follow-up study, pT217 Tau was also detected in blood plasma by mass spectrometry and found to specifically report on amyloid plaque load in the brain (Barthelemy et al., “Blood plasma phosphorylated-tau isoforms track CNS change in Alzheimer's disease”, J. Exp. Med., 2020 Vol. 217, No. 11, e20200861). Development of more accessible, cost-effective and user-friendly immunoassays that can sensitively and accurately measure pT217 Tau in human biofluids would therefore be highly desirable to enable diagnosis of AD and monitor the disease course in patients. To achieve this aim, new monoclonal antibodies that specifically recognize the pT217 Tau epitope were generated and are described herein.

### SUMMARY OF INVENTION

### Technical Problem

The object of the present invention is to provide antibodies, or antigen-binding fragments thereof, that specifically bind to pT217 Tau.

### Solution to Problem

The present invention provides the following inventions:
(1) A monoclonal antibody, or an antigen-binding fragment thereof, that specifically binds pT217 Tau, wherein the antibody comprises a heavy chain and a light chain, wherein:
the heavy chain comprises a heavy chain complementarity determining region 1 (HCDR1), a heavy chain complementarity determining region 2 (HCDR2), and a heavy chain complementarity determining region 3 (HCDR3) as set forth in SEQ ID NO: 4, and the light chain comprises a light chain complementarity determining region 1 (LCDR1), a light chain complementarity determining region 2 (LCDR2), and a light chain complementarity determining region 3 (LCDR3) as set forth in in SEQ ID NO: 9;
the heavy chain comprises a HCDR1, a HCDR2, and a HCDR3 as set forth in SEQ ID NO: 12, and the light chain comprises a LCDR1, a LCDR2, and a LCDR3 as set forth in SEQ ID NO: 15;
the heavy chain comprises a HCDR1, a HCDR2, and a HCDR3 as set forth in SEQ ID NO: 20, and the light chain comprises a LCDR1, a LCDR2, and a LCDR3 as set forth in SEQ ID NO: 23;
the heavy chain comprises a HCDR1, a HCDR2, and a HCDR3 as set forth in SEQ ID NO: 26, and the light chain comprises a LCDR1, a LCDR2, and a LCDR3 as set forth in SEQ ID NO: 30;
the heavy chain comprises a HCDR1, a HCDR2, and a HCDR3 as set forth in SEQ ID NO: 33, and the light chain comprises a LCDR1, a LCDR2, and a LCDR3 as set forth in SEQ ID NO: 35; or
the heavy chain comprises a variable domain consisting of the amino acid sequence shown in SEQ ID NO: 12, and the light chain comprises a variable domain consisting of the amino acid sequence shown in SEQ ID NO: 63.
(2) An anti-pT217 Tau antibody or a pT217 Tau-binding fragment thereof, wherein the anti-pT217 Tau antibody comprises a heavy chain and a light chain, further wherein:
(a) the heavy chain comprises a heavy chain CDR1 consisting of the amino acid sequence shown in SEQ ID NO: 1; a heavy chain CDR2 consisting of the amino acid sequence shown in SEQ ID NO: 2; and a heavy chain CDR3 consisting of the amino acid sequence shown in SEQ ID NO: 3; and the light chain comprises a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 8;
(b) the heavy chain comprises a heavy chain CDR1 consisting of the amino acid sequence shown in SEQ ID NO: 1; a heavy chain CDR2 consisting of the amino acid sequence shown in SEQ ID NO: 11; and a heavy chain CDR3 consisting of the amino acid sequence shown in SEQ ID NO: 3; and the light chain comprises a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 14;
(c) the heavy chain comprises a heavy chain CDR1 consisting of the amino acid sequence shown in SEQ ID NO: 17; a heavy chain CDR2 consisting of the amino acid sequence shown in SEQ ID NO: 18; and a heavy chain CDR3 consisting of the amino acid sequence shown in SEQ ID NO: 19; and the light chain comprises a light chain CDR1 consisting of the amino acid sequence shown in SEQ ID NO: 6; a light chain CDR2 consisting of the amino acid sequence shown in SEQ ID NO: 22; and a light chain CDR3 consisting of the amino acid sequence shown in SEQ ID NO: 8;
(d) the heavy chain comprises a heavy chain CDR 1 consisting of the amino acid sequence shown in SEQ ID NO: 1; a heavy chain CDR2 consisting of the amino acid sequence shown in SEQ ID NO: 25; and a heavy chain CDR3 consisting of the amino acid sequence shown in SEQ ID NO: 19; and the light chain comprises a light chain CDR1 consisting of the amino acid sequence shown in SEQ ID NO: 28; a light chain CDR2 consisting of the amino acid sequence shown in SEQ ID NO: 29; and a light chain CDR3 consisting of the amino acid sequence shown in SEQ ID NO: 8; or
(e) the heavy chain comprises a heavy chain CDR1 consisting of the amino acid sequence shown in SEQ ID NO: 1; a heavy chain CDR2 consisting of the amino acid sequence shown in SEQ ID NO: 32; and a heavy chain CDR3 consisting of the amino acid sequence shown in SEQ ID NO: 19; and the light chain comprises a light chain CDR1 consisting of the amino acid sequence shown in SEQ ID NO: 6; a light chain CDR2 consisting of the amino acid sequence shown in SEQ ID NO: 29; and a light chain CDR3 consisting of the amino acid sequence shown in SEQ ID NO: 8.
(3) The anti-pT217 Tau antibody or the pT217 Tau-binding fragment thereof according to (1) or (2), wherein the heavy chain comprises a heavy chain CDR1 consisting of the amino acid sequence shown in SEQ ID NO: 1; a heavy chain CDR2 consisting of the amino acid sequence shown in SEQ ID NO: 2; and a heavy chain CDR3 consisting of the amino acid sequence shown in SEQ ID NO: 3; and the light chain comprises a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 8.
(4) The anti-pT217 Tau antibody or the pT217 Tau-binding fragment thereof according to (1) or (2), wherein the heavy chain comprises a heavy chain CDR1 consisting of the amino acid sequence shown in SEQ ID NO: 1; a heavy chain CDR2 consisting of the amino acid sequence shown in SEQ ID NO: 11; and a heavy chain CDR3 consisting of the amino acid sequence shown in SEQ ID NO: 3; and the light chain comprises a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 14.
(5) The anti-pT217 Tau antibody or the pT217 Tau-binding fragment thereof according to (1) or (2), wherein the heavy chain comprises a heavy chain CDR1 consisting of the amino acid sequence shown in SEQ ID NO: 17; a heavy chain CDR2 consisting of the amino acid sequence shown in SEQ ID NO: 18; and a heavy chain CDR3 consisting of the amino acid sequence shown in SEQ ID NO: 19; and the light chain comprises a light chain CDR1 consisting of the amino acid sequence shown in SEQ ID NO: 6; a light chain CDR2 consisting of the amino acid sequence shown in SEQ ID NO: 22; and a light chain CDR3 consisting of the amino acid sequence shown in SEQ ID NO: 8.
(6) The anti-pT217 Tau antibody or the pT217 Tau-binding fragment thereof according to (1) or (2), wherein the heavy chain comprises a heavy chain CDR1 consisting of the amino acid sequence shown in SEQ ID NO: 1; a heavy chain CDR2 consisting of the amino acid sequence shown in SEQ ID NO: 25; and a heavy chain CDR3 consisting of the amino acid sequence shown in SEQ ID NO: 19; and the light chain comprises a light chain CDR1 consisting of the amino acid sequence shown in SEQ ID NO: 28; a light chain CDR2 consisting of the amino acid sequence shown in SEQ ID NO: 29; and a light chain CDR3 consisting of the amino acid sequence shown in SEQ ID NO: 8.
(7) The anti-pT217 Tau antibody or the pT217 Tau-binding fragment thereof according to (1) or (2), wherein the heavy chain comprises a heavy chain CDR1 consisting of the amino acid sequence shown in SEQ ID NO: 1; a heavy chain CDR2 consisting of the amino acid sequence shown in SEQ ID NO: 32; and a heavy chain CDR3 consisting of the amino acid sequence shown in SEQ ID NO: 19; and the light chain comprises a light chain CDR I consisting of the amino acid sequence shown in SEQ ID NO: 6; a light chain CDR2 consisting of the amino acid sequence shown in SEQ ID NO: 29; and a light chain CDR3 consisting of the amino acid sequence shown in SEQ ID NO: 8.
(8) The anti-pT217 Tau antibody or the pT217 Tau-binding fragment thereof according to (1) or (2), wherein the anti-pT217 Tau antibody comprises:
(f) a heavy chain variable domain consisting of the amino acid sequence shown in SEQ ID NO: 4, and a light chain variable domain consisting of the amino acid sequence shown in SEQ ID NO: 9;
(g) a heavy chain variable domain consisting of the amino acid sequence shown in SEQ ID NO: 12, and a light chain variable domain consisting of the amino acid sequence shown in SEQ ID NO: 15;
(h) a heavy chain variable domain consisting of the amino acid sequence shown in SEQ ID NO: 20, and a light chain variable domain consisting of the amino acid sequence shown in SEQ ID NO: 23;
(i) a heavy chain variable domain consisting of the amino acid sequence shown in SEQ ID NO: 26, and a light chain variable domain consisting of the amino acid sequence shown in SEQ ID NO: 30;
(j) a heavy chain variable domain consisting of the amino acid sequence shown in SEQ ID NO: 33, and a light chain variable domain consisting of the amino acid sequence shown in SEQ ID NO: 35; or
(k) a heavy chain variable domain consisting of the amino acid sequence shown in SEQ ID NO: 12, and a light chain variable domain consisting of the amino acid sequence shown in SEQ ID NO: 63.
(9) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 4, and a light chain variable domain consisting of the amino acid sequence shown in SEQ ID NO: 9.
(10) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 12, and a light chain variable domain consisting of the amino acid sequence shown in SEQ ID NO: 15.
(11) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 20, and a light chain variable domain consisting of the amino acid sequence shown in SEQ ID NO: 23.
(12) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 26, and a light chain variable domain consisting of the amino acid sequence shown in SEQ ID NO: 30.
(13) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 33, and a light chain variable domain consisting of the amino acid sequence shown in SEQ ID NO: 35.
(14) The anti-pT217 Tau antibody or the pT217 Tau-binding fragment thereof according to (8), wherein the anti-pT217 Tau antibody comprises a heavy chain variable domain consisting of the amino acid sequence shown in SEQ ID NO: 12, and a light chain variable domain consisting of the amino acid sequence shown in SEQ ID NO: 63.
(15) The anti-pT217 Tau antibody or the pT217 Tau-binding fragment thereof according to (1) or (2), wherein the anti-pT217 Tau antibody comprises:
(1) a heavy chain comprising the amino acid sequence shown in SEQ ID NO: 5, and a light chain comprising the amino acid sequence shown in SEQ ID NO: 10;
(m) a heavy chain comprising the amino acid sequence shown in SEQ ID NO: 13, and a light chain comprising the amino acid sequence shown in SEQ ID NO: 16;
(n) a heavy chain comprising the amino acid sequence shown in SEQ ID NO: 21, and a light chain comprising the amino acid sequence shown in SEQ ID NO: 24;
(o) a heavy chain comprising the amino acid sequence shown in SEQ ID NO: 27, and a light chain comprising the amino acid sequence shown in SEQ ID NO: 31;
(p) a heavy chain comprising the amino acid sequence shown in SEQ ID NO: 34, and a light chain comprising the amino acid sequence shown in SEQ ID NO: 36; or
(q) a heavy chain comprising the amino acid sequence shown in SEQ ID NO: 13, and a light chain comprising the amino acid sequence shown in SEQ ID NO: 64.
(16) The anti-pT217 Tau antibody or the pT217 Tau-binding fragment thereof according to (15), wherein the heavy chain comprises the amino acid sequence shown in SEQ ID NO: 5, and the light chain comprises the amino acid sequence shown in SEQ ID NO: 10.
(17) The anti-pT217 Tau antibody or the pT217 Tau-binding fragment thereof according to (15), wherein the heavy chain comprises the amino acid sequence shown in SEQ ID NO: 13, and the light chain comprises the amino acid sequence shown in SEQ ID NO: 16.
(18) The anti-pT217 Tau antibody or the pT217 Tau-binding fragment thereof according to (15), wherein the heavy chain comprises the amino acid sequence shown in SEQ ID NO: 21, and the light chain comprises the amino acid sequence shown in SEQ ID NO: 24.
(19) The anti-pT217 Tau antibody or the pT217 Tau-binding fragment thereof according to (15), wherein the heavy chain comprises the amino acid sequence shown in SEQ ID NO: 27, and the light chain comprises the amino acid sequence shown in SEQ ID NO: 31.
(20) The anti-pT217 Tau antibody or the pT217 Tau-binding fragment thereof according to (15), wherein the heavy chain comprises the amino acid sequence shown in SEQ ID NO: 34, and the light chain comprises the amino acid sequence shown in SEQ ID NO: 36.
(21) The anti-pT217 Tau antibody or the pT217 Tau-binding fragment thereof according to (15), wherein the heavy chain comprises the amino acid sequence shown in SEQ ID NO: 13, and the light chain comprises the amino acid sequence shown in SEQ ID NO: 64.
(22) An isolated nucleic acid encoding the anti-pT217 Tau antibody or the pT217 Tau-binding fragment thereof according to any one of (1) to (21).
(23) An expression vector comprising the nucleic acid according to (22).
(24) A host cell comprising the expression vector according to (23).
(25) A method of producing an anti-pT217 Tau antibody or a pT217 Tau-binding fragment thereof, comprising a step of culturing the host cell according to (24).
(26) A method for detecting the presence or amount of pT217 Tau in a biological sample, comprising:
contacting the biological sample with the anti-pT217 Tau antibody or the pT217 Tau-binding fragment thereof according to any one of (1) to (21); and detecting the presence or amount of pT217 Tau in the biological sample.
(27) A method for detecting the presence or amount of pT217 Tau in a biological sample, comprising:
contacting the biological sample with the anti-pT217 Tau antibody or the pT217 Tau-binding fragment thereof according to any one of (1) to (21); and detecting the presence or amount of complex formed between the anti-pT217 Tau antibody or pT217 Tau-binding fragment and pT217 Tau in the biological sample.
(28) The method according to (26) or (27), wherein the biological sample is blood, serum, plasma, or cerebral spinal fluid.
(29) The method according to any one of (26) to (28) wherein the biological sample is obtained from a subject that suffers from or is at risk of suffering from Alzheimer's disease or mild cognitive impairment due to Alzheimer's disease.

The present invention also provides the following inventions.
(30) A method for diagnosing Alzheimer's disease or mild cognitive impairment due to Alzheimer's disease in a subject, comprising contacting a biological sample from the subject with the anti-pT217 Tau antibody or the pT217 Tau-binding fragment thereof according to any one of (1) to (21); and detecting the presence or amount of pT217 Tau in the biological sample.
(31) The method according to (30), wherein the subject is diagnosed as having Alzheimer's disease or mild cognitive impairment due to Alzheimer's disease when the presence or certain amount of pT217 Tau in the biological sample is detected.
(32) A method for diagnosing Alzheimer's disease or mild cognitive impairment due to Alzheimer's disease in a subject, comprising contacting a biological sample from the subject with the anti-pT217 Tau antibody or the pT217 Tau-binding fragment thereof according to any one of (1) to (21); and detecting the presence or amount of complex formed between the anti-pT217 Tau antibody or pT217 Tau-binding fragment and pT217 Tau in the biological sample.
(33) The method according to (32), wherein the subject is diagnosed as having Alzheimer's disease or mild cognitive impairment due to Alzheimer's disease when the presence or certain amount of complex formed between the anti-pT217 Tau antibody or pT217 Tau-binding fragment and pT217 Tau in the biological sample is detected.
(34) A method for aiding diagnosis of Alzheimer's disease or mild cognitive impairment due to Alzheimer's disease in a subject, comprising contacting a biological sample from the subject with the anti-pT217 Tau antibody or the pT217 Tau-binding fragment thereof according to any one of (1) to (21); and detecting the presence or amount of pT217 Tau in the biological sample.
(35) The method according to (34), wherein the subject is diagnosed as having Alzheimer's disease or mild cognitive impairment due to Alzheimer's disease when the present or certain amount of pT217 Tau in the biological sample is detected.
(36) A method for aiding diagnosis of Alzheimer's disease or mild cognitive impairment due to Alzheimer's disease in a subject, comprising contacting a biological sample from the subject with the anti-pT217 Tau antibody or the pT217 Tau-binding fragment thereof according to any one of (1) to (21); and detecting the presence or amount of complex formed between the anti-pT217 Tau antibody or pT217 Tau-binding fragment and pT217 Tau in the biological sample.
(37) The method according to (36), wherein the subject is diagnosed as having Alzheimer's disease or mild cognitive impairment due to Alzheimer's disease when the present or certain amount of complex formed between the anti-pT217 Tau antibody or pT217 Tau-binding fragment and pT217 Tau in the biological sample is detected.
(38) A method for detecting the presence or amount of pT217 Tau for diagnosing Alzheimer's disease or mild cognitive impairment due to Alzheimer's disease in a subject, comprising:
contacting a biological sample from the subject with the anti-pT217 Tau antibody or the pT217 Tau-binding fragment thereof according to any one of (1) to (21); and detecting the presence or amount of pT217 Tau in the biological sample.
(39) The method according to (38), wherein the subject is diagnosed as having Alzheimer's disease or mild cognitive impairment due to Alzheimer's disease when the present or certain amount of pT217 Tau in the biological sample is detected.
(40) A method for detecting the presence or amount of pT217 Tau for diagnosing Alzheimer's disease or mild cognitive impairment due to Alzheimer's disease in a subject, comprising:
contacting a biological sample from the subject with the anti-pT217 Tau antibody or the pT217 Tau-binding fragment thereof according to any one of (1) to (21); and detecting the presence or amount of complex formed between the anti-pT217 Tau antibody or pT217 Tau-binding fragment and pT217 Tau in the biological sample.
(41) The method according to (40), wherein the subject is diagnosed as having Alzheimer's disease or mild cognitive impairment due to Alzheimer's disease when the present or certain amount of complex formed between the anti-pT217 Tau antibody or pT217 Tau-binding fragment and pT217 Tau in the biological sample is detected.
(42) A method for evaluating amyloid beta accumulation in a subject, comprising: contacting a biological sample from the subject with the anti-pT217 Tau antibody or the pT217 Tau-binding fragment thereof according to any one of (1) to (21); and detecting the presence or amount of pT217 Tau in the biological sample.
(43) The method according to (42), wherein the subject is determined as having amyloid beta accumulation when the presence or certain amount of pT217 Tau in the biological sample is detected.
(44) A method for evaluating amyloid beta accumulation in a subject, comprising: contacting a biological sample from the subject with the anti-pT217 Tau antibody or the pT217 Tau-binding fragment thereof according to any one of (1) to (21); and detecting the presence or amount of complex formed between the anti-pT217 Tau antibody or pT217 Tau-binding fragment and pT217 Tau in the biological sample.
(45) The method according to (44), wherein the method further comprises comparing the amount of complex in the biological sample to an amount of complex formed in a control biological sample.
(46) The method according to (44) or (45), wherein the subject is determined as having amyloid beta accumulation when the presence or certain amount of complex formed between the anti-pT217 Tau antibody or pT217 Tau-binding fragment and pT217 Tau in the biological sample is detected.
(47) The method according to any one of (30) to (45), wherein the biological sample is blood, serum, plasma, or cerebral spinal fluid.
(48) The method according to any one of (30) to (46), wherein the subject suffers from or is at risk of suffering from Alzheimer's disease or mild cognitive impairment due to Alzheimer's disease.
(49) A kit for detecting the presence or amount of pT217 Tau, comprising the antipT217 Tau antibody or the pT217 Tau-binding fragment thereof according to any one of (1) to (21).
(50) A kit for diagnosing Alzheimer's disease or mild cognitive impairment due to Alzheimer's disease, comprising the anti-pT217 Tau antibody or the pT217 Tau-binding fragment thereof according to any one of (1) to (21).
(51) A kit for evaluating amyloid beta accumulation, comprising the anti-pT217 Tau antibody or the pT217 Tau-binding fragment thereof according to any one of (1) to (21).

### BRIEF DESCRIPTION OF DRAWINGS

FIG. 1 shows the results of inhibition ELISA using p-Tau protein in Example 2.

FIG. 2 shows the results of inhibition ELISA using p-Tau protein in Example 2.

FIG. 3 shows the results of inhibition ELISA using p-Tau protein in Example 2.

FIG. 4 shows the results of inhibition ELISA using p-Tau protein in Example 2.

FIG. 5 shows the results of inhibition ELISA using Alzheimer's Disease brain lysate in Example 3.

FIG. 6 shows the results of inhibition ELISA using Alzheimer's Disease brain lysate in Example 3.

FIG. 7 shows the results of inhibition ELISA using Alzheimer's Disease brain lysate in Example 3.

FIG. 8 shows the results of inhibition ELISA using Alzheimer's Disease brain lysate in Example 3.

FIG. 9 shows the results of Western blot analysis in Example 4.

FIG. 10 shows the results of Western blot analysis in Example 4.

FIG. 11 shows the results of Western blot analysis in Example 4.

FIG. 12 shows the results of reactivity to pT217 Tau peptide of sandwich ELISA in Example 7.

FIG. 13 shows the results of selectivity to pT217 Tau peptide of sandwich ELISA in Example 8.

FIG. 14 shows the results of selectivity to pT217 Tau peptide of sandwich ELISA in Example 8.

FIG. 15 shows the results of selectivity to pT217 Tau peptide of sandwich ELISA in Example 8.

FIG. 16 shows the results of selectivity to pT217 Tau peptide of sandwich ELISA in Example 8.

FIG. 17 shows the results of selectivity to pT217 Tau peptide of sandwich ELISA in Example 8.

FIG. 18 shows the results of selectivity to pT217 Tau peptide of sandwich ELISA in Example 8.

FIG. 19 shows the results of selectivity to pT217 Tau peptide of inhibition test in sandwich ELISA in Example 9.

FIG. 20 shows the results of selectivity to pT217 Tau peptide of inhibition test in sandwich ELISA in Example 9.

FIG. 21 shows the results of selectivity to pT217 Tau peptide of inhibition test in sandwich ELISA in Example 9.

FIG. 22 shows the results of selectivity to pT217 Tau peptide of inhibition test in sandwich ELISA in Example 9.

FIG. 23 shows the results of selectivity to pT217 Tau peptide of inhibition test in sandwich ELISA in Example 9.

FIG. 24 shows the results of selectivity to pT217 Tau peptide of inhibition test in sandwich ELISA in Example 9.

FIG. 25 shows the results of selectivity to pT217 Tau peptide of inhibition test in sandwich ELISA in Example 9.

FIG. 26 shows the results of selectivity to pT217 Tau peptide of inhibition test in sandwich ELISA in Example 9.

FIG. 27 shows biotinylated non-phosphorylated and phosphorylated tau peptides used in Example 11.

FIG. 28 shows pT217 Tau level in human CSF (AD samples) in Example 12.

FIG. 29 shows pT217 Tau level in human CSF (MCI samples) in Example 12.

FIG. 30 shows pT217 Tau level in human CSF (control samples) in Example 12.

FIG. 31 shows pT217 Tau level in human CSF (all samples) in Example 12.

FIG. 32 shows pT181 Tau level in human CSF (all samples) in Example 12.

FIG. 33 shows correlation between pT217 Tau and pT181 Tau levels in human CSF (all samples) in Example 12.

FIG. 34 shows pT217 Tau level in human plasma (AD and control samples) in Example 13.

FIGS. 35-38 show temporal accumulation of sarkosyl-insoluble total tau and pT217 tau in an in vivo model of tau seeding and transmission in Example 14. Insoluble fraction from AD brain containing tau seeds was injected into the left side (ipsilateral) hippocampi of either hTau mice or their KO littermate controls. Animals were sacrificed post seed-injection at either 1, 6, or 12 weeks (as indicated) and the sarkosyl-insoluble fraction extracted from the ipsilateral (FIGS. 35 and 36) and contralateral (FIGS. 37 and 38) hippocampus. Total tau (FIGS. 35 and 36) and pT217 tau (FIGS. 37 and 38) were quantified by western blotting using the K9JA and 5F6-4D1 antibodies respectively.

FIGS. 35-38 show temporal accumulation of sarkosyl-insoluble total tau and pT217 tau in an in vivo model of tau seeding and transmission in Example 14. Insoluble fraction from AD brain containing tau seeds was injected into the left side (ipsilateral) hippocampi of either hTau mice or their KO littermate controls. Animals were sacrificed post seed-injection at either 1, 6, or 12 weeks (as indicated) and the sarkosyl-insoluble fraction extracted from the ipsilateral (FIGS. 35 and 36) and contralateral (FIGS. 37 and 38) hippocampus. Total tau (FIGS. 35 and 36) and pT217 tau (FIGS. 37 and 38) were quantified by western blotting using the K9JA and 5F6-4D1 antibodies respectively.

FIGS. 35-38 show temporal accumulation of sarkosyl-insoluble total tau and pT217 tau in an in vivo model of tau seeding and transmission in Example 14. Insoluble fraction from AD brain containing tau seeds was injected into the left side (ipsilateral) hippocampi of either hTau mice or their KO littermate controls. Animals were sacrificed post seed-injection at either 1, 6, or 12 weeks (as indicated) and the sarkosyl-insoluble fraction extracted from the ipsilateral (FIGS. 35 and 36) and contralateral (FIGS. 37 and 38) hippocampus. Total tau (FIGS. 35 and 36) and pT217 tau (FIGS. 37 and 38) were quantified by western blotting using the K9JA and 5F6-4D1 antibodies respectively.

FIGS. 35-38 show temporal accumulation of sarkosyl-insoluble total tau and pT217 tau in an in vivo model of tau seeding and transmission in Example 14. Insoluble fraction from AD brain containing tau seeds was injected into the left side (ipsilateral) hippocampi of either hTau mice or their KO littermate controls. Animals were sacrificed post seed-injection at either 1, 6, or 12 weeks (as indicated) and the sarkosyl-insoluble fraction extracted from the ipsilateral (FIGS. 35 and 36) and contralateral (FIGS. 37 and 38) hippocampus. Total tau (FIGS. 35 and 36) and pT217 tau (FIGS. 37 and 38) were quantified by western blotting using the K9JA and 5F6-4D1 antibodies respectively.

### DESCRIPTION OF EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a plurality of such cells, reference to “a heavy chain” includes a plurality of such heavy chains, and reference to “a light chain” includes a plurality of such light chains, and the like.

The term “about” as used herein when referring to a measurable value such as an amount, a temporal duration, and the like, is meant to encompass variations of up to +10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

“Isolated” means a biological component (such as a nucleic acid, peptide or protein) has been substantially separated, produced apart from, or purified away from other biological components of the organism in which the component naturally occurs, i.c., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins that can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

“Polynucleotide,” synonymously referred to as “nucleic acid molecule” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single-and double-stranded regions. “Polynucleotide” also embraces relatively short nucleic acid chains, often referred to as oligonucleotides.

A “vector” is a replicon, such as plasmid, phage, cosmid, or virus in which another nucleic acid segment may be operably inserted so as to bring about the replication or expression of the segment.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression or production of an antibody or antigen-binding fragment thereof may be within the cytoplasm of the cell, or into the extracellular milieu such as the growth medium of a cell culture.

The term “antibody” as used herein is meant in a broad sense and includes immunoglobulin or antibody molecules including polyclonal antibodies, monoclonal antibodies including murine, human, human-adapted, humanized and chimeric monoclonal antibodies and antibody fragments. In general, antibodies are proteins or peptide chains that exhibit binding specificity to a specific antigen. Intact antibodies are heterotetrameric glycoproteins, composed of two identical light chains and two identical heavy chains. Typically, cach light chain is linked to a heavy chain by onc covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (variable region) (VH) followed by a number of constant domains (constant regions). Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains.

Immunoglobulins can be assigned to five major classes or isotypes, depending upon the type of constant domain possessed by its heavy chain, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. The heavy chain constant domains that correspond to the different classes of immunoglobulins are called alpha, delta, epsilon, gamma, and mu, respectively.

An immunoglobulin light chain variable region or heavy chain variable region consists of a “framework” region interrupted by three “antigen-binding sites”. The antigen-binding sites are defined using various terms as follows: (i) the term Complementarity Determining Regions (CDRs) is based on sequence variability (Wu and Kabat, J. Exp. Med. 132:211-250, 1970). Generally, the antigen-binding site has six CDRs; three in the VH (HCDR1, HCDR2, HCDR3), and three in the VL (LCDR1, LCDR2, LCDR3) (Kabat et al., Sequences of Proteins of Immunological Interest, 5th Ed. Public Health Service, National Institutes of Health, Bethesda, Md., 1991). The “IMGT-CDRs” as proposed by Lefranc (Lefranc et al., Dev. Comparat. Immunol. 27:55-77, 2003) are based on the comparison of V domains from immunoglobulins and T-cell receptors. The International ImMunoGeneTics (IMGT) database (http://www_imgt_org) provides a standardized numbering and definition of these regions. The correspondence between CDRs and IMGT delineations is described in Lefranc et al., Dev. Comparat. Immunol. 27:55-77, 2003.

Antigen-binding fragments are any proteinaceous structure that may exhibit binding affinity for a particular antigen. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)₂, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, and the like. All antibody isotypes may be used to produce antigen-binding fragments. Additionally, antigen-binding fragments may include non-antibody proteinaccous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase.

“Specific binding to pT217 Tau” or “specifically binds to pT217 Tau” refers to the binding of an antibody or antigen-binding fragment to Tau at epitope comprising a phosphorylated threonine 217. In some embodiments, the anti-pT217 Tau antibody or the pT217 Tau-binding fragment thereof specifically binds to pT217 Tau with greater affinity than for non-phosphorylated Tau.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

Human Tau 2N4R (also referred to as Tau441) is
set forth herein as SEQ ID NO: 67:
MAEPRQEFEVMEDHAGTYGLGDRKDQGGYTMHQDQEGDTDAGLKESPLQ
TPTEDGSEEPGSETSDAKSTPTAEDVTAPLVDEGAPGKQAAAQPHTEIP
EGTTAEEAGIGDTPSLEDEAAGHVTQARMVSKSKDGTGSDDKKAKGADG
KTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQT
APVPMPDLKNVKSKIGSTENLKHQPGGGKVQIINKKLDLSNVQSKCGSK
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAEI
VYKSPVVSGDTSPRHLSNVSSTGSIDMVDSPQLATLADEVSASLAKQG
L.

Human Tau also refers to Tau variants, for example, naturally occurring allelic variants, including 2N4R (UniProt Acc. No. P10636-8); 1N4R (UniProt Acc. No. P10636-7); ON4R (UniProt Acc. No. P10636-6); 2N3R (UniProt Acc. No. P10636-5); 1N3R (UniProt Acc. No. P10636-4); and ON3R (UniProt Acc. No. P10636-2), or sequences containing at least one amino acid substitution relative thereto.

pT217 Tau is Tau 2N4R phosphorylated at threonine 217 and also includes 1N3R phosphorylated at threonine 188, ON4R phosphorylated at threonine 159, 2N3R phosphorylated at threonine 217, 1N3R phosphorylated at threonine 188, and ON3R phosphorylated at threonine 157.

In some embodiments, the anti-pT217 Tau antibodies or the pT217 Tau-binding fragments thereof are murine IgG, or derivatives thereof. While the anti-pT217 Tau antibodies or the pT217 Tau-binding fragments thereof may be human, humanized, or chimeric, the anti-pT217 Tau antibodies or the pT217 Tau-binding fragments thereof exemplified herein are murine antibodies.

In some embodiments described herein, the heavy chain constant domain of the anti-pT217 Tau antibody or the pT217 Tau-binding fragment is IgG2. In certain embodiments, the heavy chain constant domain of the anti-pT217 Tau antibody or the pT217 Tau-binding fragment is mouse IgG2b, and the light chain constant domain of the anti-pT217 Tau antibody or the pT217 Tau-binding fragment is mouse kappa. The anti-pT217 Tau antibodies or the pT217 Tau-binding fragments thereof as disclosed in the examples section are derived from mice. Similar antibodies may be derived from any species by recombinant means. For example, the anti-pT217 Tau antibodies or the pT217 Tau-binding fragments thereof may be chimeric rat, goat, horse, swine, bovine, chicken, rabbit, camelid, donkey, human, and the like.

In some embodiments, the anti-pT217 Tau antibodies or the pT217 Tau-binding fragments thereof are chimeric. As used herein, the term “chimeric” refers to an antibody, or antigen-binding fragment thercof, having at least some portion of at least one variable domain derived from the antibody amino acid sequence of a non-human mammal, a rodent, or a reptile, while the remaining portions of the antibody, or antigen-binding fragment thereof, are derived from a human. For example, a chimeric antibody may comprise a mouse antigen binding domain with a human Fc or other such structural domain.

In some embodiments, the anti-pT217 Tau antibodies or the pT217 Tau-binding fragments thereof arc humanized antibodies or fragments. Humanized antibodies may be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin.

The anti-pT217 Tau antibodies or the pT217 Tau-binding fragments thereof described herein can occur in a variety of forms, but will include one or more of the antibody variable domain segments or CDRs shown in Tables 2-4 in Examples.

In some embodiments, the anti-pT217 Tau antibodies or the pT217 Tau-binding fragments thereof comprise heavy and light chains, and comprise:
(a) a heavy chain CDR1 consisting of the amino acid sequence shown in SEQ ID
NO: 1; a heavy chain CDR2 consisting of the amino acid sequence shown in SEQ ID NO: 2; a heavy chain CDR3 consisting of the amino acid sequence shown in SEQ ID NO: 3; a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 8;
(b) a heavy chain CDR1 consisting of the amino acid sequence shown in SEQ ID NO: 1; a heavy chain CDR2 consisting of the amino acid sequence shown in SEQ ID NO: 11; a heavy chain CDR3 consisting of the amino acid sequence shown in SEQ ID NO: 3; a light chain CDR1 consisting of the amino acid sequence shown in SEQ ID NO: 6; a light chain CDR2 consisting of the amino acid sequence shown in SEQ ID NO: 7; and a light chain CDR3 consisting of the amino acid sequence shown in SEQ ID NO: 14;
(c) a heavy chain CDR1 consisting of the amino acid sequence shown in SEQ ID NO: 17; a heavy chain CDR2 consisting of the amino acid sequence shown in SEQ ID NO: 18; a heavy chain CDR3 consisting of the amino acid sequence shown in SEQ ID NO: 19; a light chain CDR I consisting of the amino acid sequence shown in SEQ ID NO: 6; a light chain CDR2 consisting of the amino acid sequence shown in SEQ ID NO: 22; and a light chain CDR3 consisting of the amino acid sequence shown in SEQ ID NO: 8;
(d) a heavy chain CDR1 consisting of the amino acid sequence shown in SEQ ID NO: 1; a heavy chain CDR2 consisting of the amino acid sequence shown in SEQ ID NO: 25; a heavy chain CDR3 consisting of the amino acid sequence shown in SEQ ID NO: 19; a light chain CDR1 consisting of the amino acid sequence shown in SEQ ID NO: 28; a light chain CDR2 consisting of the amino acid sequence shown in SEQ ID NO: 29; and a light chain CDR3 consisting of the amino acid sequence shown in SEQ ID NO: 8; or
(e) a heavy chain CDR1 consisting of the amino acid sequence shown in SEQ ID NO: 1; a heavy chain CDR2 consisting of the amino acid sequence shown in SEQ ID NO: 32; a heavy chain CDR3 consisting of the amino acid sequence shown in SEQ ID NO: 19; a light chain CDR1 consisting of the amino acid sequence shown in SEQ ID NO: 6; a light chain CDR2 consisting of the amino acid sequence shown in SEQ ID NO: 29; and a light chain CDR3 consisting of the amino acid sequence shown in SEQ ID NO: 8.

In some embodiments, the anti-pT217 Tau antibodies or the pT217 Tau-binding fragments thereof comprise heavy and light chains, and comprise:
(f) a heavy chain variable domain consisting of the amino acid sequence shown in SEQ ID NO: 4, and a light chain variable domain consisting of the amino acid sequence shown in SEQ ID NO: 9;
(g) a heavy chain variable domain consisting of the amino acid sequence shown in SEQ ID NO: 12, and a light chain variable domain consisting of the amino acid sequence shown in SEQ ID NO: 15;
(h) a heavy chain variable domain consisting of the amino acid sequence shown in SEQ ID NO: 20, and a light chain variable domain consisting of the amino acid sequence shown in SEQ ID NO: 23;
(i) a heavy chain variable domain consisting of the amino acid sequence shown in SEQ ID NO: 26, and a light chain variable domain consisting of the amino acid sequence shown in SEQ ID NO: 30;
(j) a heavy chain variable domain consisting of the amino acid sequence shown in SEQ ID NO: 33, and a light chain variable domain consisting of the amino acid sequence shown in SEQ ID NO: 35; or
(k) a heavy chain variable domain consisting of the amino acid sequence shown in SEQ ID NO: 12, and a light chain variable domain consisting of the amino acid sequence shown in SEQ ID NO: 63.

In some embodiments, the anti-pT217 Tau antibody or the pT217 Tau-binding fragment thereof comprise heavy and light chains, and comprise:
(l) a heavy chain comprising the amino acid sequence shown in SEQ ID NO: 5, and a light chain comprising the amino acid sequence shown in SEQ ID NO: 10;
(m) a heavy chain comprising the amino acid sequence shown in SEQ ID NO: 13, and a light chain comprising the amino acid sequence shown in SEQ ID NO: 16;
(n) a heavy chain comprising the amino acid sequence shown in SEQ ID NO: 21, and a light chain comprising the amino acid sequence shown in SEQ ID NO: 24;
(o) a heavy chain comprising the amino acid sequence shown in SEQ ID NO: 27, and a light chain comprising the amino acid sequence shown in SEQ ID NO: 31;
(p) a heavy chain comprising the amino acid sequence shown in SEQ ID NO: 34, and a light chain comprising the amino acid sequence shown in SEQ ID NO: 36; or
(q) a heavy chain comprising the amino acid sequence shown in SEQ ID NO: 13, and a light chain comprising the amino acid sequence shown in SEQ ID NO: 64.

The anti-pT217 Tau antibodies or the pT217 Tau-binding fragments thereof include variants having single or multiple amino acid substitutions, deletions, or additions that retain the biological properties (e.g., binding affinity or immune effector activity) of the described antibodies or antigen-binding fragments. The skilled person may produce variants having single or multiple amino acid substitutions, delctions, or additions. These variants may include: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidinc sequence, a biotin moiety and the like. The anti-pT217 Tau antibodies or the pT217 Tau-binding fragments thereof may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (suppressions, deletions, mutations, etc.), chemical, and enzymatic techniques, are known to the person having ordinary skill in the art.

In certain embodiments, labelled anti-pT217 Tau antibodies or pT217 Tau-binding fragments thereof are provided. Labels include, but are not limited to, labels or moieties that are detected directly (e.g., fluorescent, chromophoric, electron-dense, chemiluminescent, and radioactive labels) and labels and moieties (e.g., enzymes or ligands) that are detected indirectly (e.g., through enzymatic reaction or molecular interaction). Exemplary labels include but are not limited to radiolabels (e.g., ³²P, ¹¹C, ¹⁴C, ¹¹¹I, ¹²⁵I, ³H, ¹³¹I, ¹⁸F), fluorescent labels (such as DyLight™ 649), epitope tags, biotin, chromophore labels, ECL labels, or enzymes. More specifically, the described labels include ruthenium, ¹¹¹In-DOTA, ¹¹¹In-diethylenetriaminepentaacetic acid (DTPA), horseradish peroxidase, alkaline phosphatase and beta-galactosidase, polyhistidine (HIS tag), acridine dyes, cyanine dyes, fluorone dyes, oxazin dyes, phenanthridine dyes, rhodamine dyes, Alexafluor™dyes, and the like.

Also disclosed are isolated nucleic acids that encode the anti-pT217 Tau antibodies or the pT217 Tau-binding fragments thereof. In some embodiments, the isolated nucleic acids encode the anti-pT217 Tau antibodies or the pT217 Tau-binding fragments thereof according to any one of (1) to (21) above.

The isolated nucleic acid provided herein refers to one or more nucleic acid molecules encoding the heavy chain and/or light chain of the anti-pT217 Tau antibody or the pT217 Tau-binding fragment thereof. In some embodiments, the isolated nucleic acid encodes the heavy chain of the anti-pT217 Tau antibody or the pT217 Tau-binding fragment thereof; the light chain of the anti-pT217 Tau antibody or the pT217 Tau-binding fragment thereof; or the heavy and light chains of the anti-pT217 Tau antibody or the pT217 Tau-binding fragment thereof. In certain embodiments, the isolated nucleic acid comprises a first nucleic acid molecule encoding the heavy chain of the anti-pT217 Tau antibody or the pT217 Tau-binding fragment thereof and a second nucleic acid molecule encoding the light chain of the anti-pT217 Tau antibody or the pT217 Tau-binding fragment thereof.

The nucleic acids capable of encoding the variable domain segments provided herein may be included on the same, or different, vectors to produce the anti-pT217 Tau antibodies or the pT217 Tau-binding fragments thereof. Nucleic acids encoding engineered antigen-binding proteins also are within the scope of the disclosure. In some embodiments, the nucleic acids described (and the peptides they encode) include a leader sequence. Any leader sequence known in the art may be employed. The leader sequence may include, but is not limited to, a restriction site or a translation start site. Also provided are vectors comprising the nucleic acids described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors such as retroviral, lentiviral, adenoviral, adeno-associated viral, and herpes simplex viral vector.

The vector provided herein refers to one or more vectors comprising the isolated nucleic acid encoding the anti-pT217 Tau antibody or the pT217 Tau-binding fragment thereof. In some embodiments, the vector is a vector comprising the nucleic acid encoding the heavy chain of the anti-pT217 Tau antibody or the pT217 Tau-binding fragment thereof and the nucleic acid encoding the light chain of the anti-pT217 Tau antibody or the pT217 Tau-binding fragment thereof, or a vector comprising the nucleic acid encoding the heavy and light chains of the anti-pT217 Tau antibody or the pT217 Tau-binding fragment thereof. In certain embodiments, the vector comprises a first vector comprising the nucleic acid encoding the heavy chain of the anti-pT217 Tau antibody or the pT217 Tau-binding fragment thereof and a second vector comprising the nucleic acid encoding the light chain of the anti-pT217 Tau antibody or the pT217 Tau-binding fragment thereof.

Recombinant expression vectors within the scope of the description include synthetic, genomic, or cDNA-derived nucleic acid fragments that encode at least one recombinant protein which may be operably linked to suitable regulatory elements. Such regulatory clements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the nucleic acid encoding the anti-pT217 Tau antibody or the pT217 Tau-binding fragment thereof is placed under control of a powerful constitutive promoter, such as the promoter for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate carly promoter, the carly and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the nucleic acid encoding the anti-pT217 Tau antibody or the pT217 Tau-binding fragment thereof is placed under control of an inducible promoter such as the metallothionein promoter, tetracycline-inducible promoter, doxycycline-inducible promoter, promoter that contains one or more interferon-stimulated response elements (ISRE) such as protein kinase R 2′,5′-oligoadenylate synthetases, Mx genes, ADARI, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate antibody or antigen-binding fragment-producing cells. Thus, another aspect features host cells transformed with vectors comprising a nucleic acid sequence encoding the anti-pT217 Tau antibodies or the pT217 Tau-binding fragments thereof.

Numerous techniques are known in the art for the introduction of foreign genes into cells and may be used to construct the recombinant cells for purposes of carrying out the described methods in accordance with the various embodiments described and examplified herein. The technique used should provide for the stable transfer of the hetcrologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the anti-pT217 Tau antibodies or the pT217 Tau-binding fragments thereof are preferably eukaryotic cells, more preferably cells of plant, rodent, or human origin, for example but not limited to NS0, CHO, CHOK1, perC.6, Tk-ts13, BHK, HEK293 cells, COS-7, T98G, CV-1/EBNA, L cells, C127, 3T3, HeLa, NS1, and Sp2/0 myeloma cells cell lines, among others. In addition, expression of antibodies may be accomplished using hybridoma cells. Methods for producing hybridomas are well established in the art.

Cells transformed with expression vectors described herein may be selected or screened for recombinant expression of the anti-pT217 Tau antibodies or the pT217 Tau-binding fragments thereof. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

In certain embodiments, an isolated cell line expressing any of the anti-pT217 Tau antibodies or the pT217 Tau-binding fragments thereof is provided. In one embodiment, the isolated cell line is a hybridoma.

Cells that express the anti-pT217 Tau antibodies or the pT217 Tau-binding fragments thereof may be employed in methods of producing the anti-pT217 Tau antibodies or the pT217 Tau-binding fragments thereof by culturing the cells under conditions suitable for expression of the respective anti-pT217 Tau antibodies or pT217 Tau-binding fragments thereof. In some embodiments, the anti-pT217 Tau antibodies or the pT217 Tau-binding fragments thereof is recovered from the culture medium.

Also provided herein are methods of preparing a sample, wherein the method comprises contacting a biological sample with the anti-pT217 Tau antibody or the pT217 Tau-binding fragment under conditions permissive for binding of the antibody or antigen-binding fragment to pT217 Tau. In some embodiments of these methods, the anti-pT217 Tau antibody or anti-pT217 Tau-binding fragment is detectably labelled. The method may be an in vitro or in vivo method. In some embodiments, the complex formed between the anti-pT217 Tau antibody or the pT217 Tau-binding fragment and pT217 Tau is isolated.

In certain embodiments, any of the anti-pT217 Tau antibodies or the pT217 Tau-binding fragments thereof is useful for detecting the presence or amount of pT217 Tau in a biological sample from a subject. The term “detecting” as used herein encompasses quantitative or qualitative detection. Methods of detecting the presence or amount of protein are well known in the art. The methods of detecting the presence or amount of pT217 Tau include, but are not limited to, immunocytochemistry, enzyme-linked immunosorbent assay (ELISA), immunohistochemistry, radioimmunoassay, western blot or dot blot analysis, enzyme-linked immunosorbent spot (ELISPOT), nuclear medicine imaging (e.g., SPECT, PET), other in-vivo imaging, etc. In certain embodiments, the biological sample may be derived from a cell or tissue of the body fluid, such as blood, serum, plasma, cerebral spinal fluid, urine, saliva, lacrimal fluid or sweat, or a cell or tissue of the brain (e.g., cortex or hippocampus), a histological preparation, and the like. In certain embodiments, the body fluid may be blood, serum, plasma, or cerebral spinal. In some embodiments, the subject suffers from or is at risk of suffering from Alzheimer's disease or mild cognitive impairment due to Alzheimer's disease (MCI due to AD). In some embodiments the described methods include detecting the presence or amount of pT217 Tau by contacting the biological sample with the anti-pT217 Tau antibodies or pT217 Tau-binding fragments thereof according to any one or more of (1) to (21) above.

In certain embodiments, the method comprises contacting the biological sample with the anti-pT217 Tau antibody or the pT217 Tau-binding fragment thereof under conditions permissive for binding of the antibody or antigen-binding fragment to pT217 Tau, and detecting whether a complex is formed between the anti-pT217 Tau antibody or anti-pT217 Tau-binding fragment and pT217 Tau. In some embodiments of these methods, the anti-pT217 Tau antibody or anti-pT217 Tau-binding fragment is detectably labelled. The method may be an in vitro or in vivo method. The complex formed between the anti-pT217 Tau antibody or anti-pT217 Tau-binding fragment and pT217 Tau in a test biological sample can be compared to the complex formed in a control biological sample (e.g., a biological sample from a healthy subject). The amount of the complex formed between anti-pT217 Tau antibody or anti-pT217 Tau-binding fragment and pT217 Tau in a test biological sample can also be quantified and compared to the amount of the complex formed in a control biological sample or to the average amount of the complex known to be formed in healthy subjects. The healthy subject may be an amyloid-negative subject not having amyloid beta accumulation. The amount of the complex formed between the anti-pT217 Tau antibody or anti-pT217 Tau-binding fragment and pT217 Tau in a test biological sample can also be quantified and compared to a control. In certain embodiments, the control is a predetermined cut-off value.

In certain embodiments, any of the anti-pT217 Tau antibodies or anti-pT217 Tau-binding fragments is useful for diagnosing Alzheimer's disease or mild cognitive impairment due to Alzheimer's disease in a subject, aiding diagnosis of Alzheimer's disease or mild cognitive impairment due to Alzheimer's disease in a subject, detecting the presence or amount of pT217 Tau for diagnosing Alzheimer's disease or mild cognitive impairment due to Alzheimer's disease in a subject, and evaluating amyloid beta accumulation. In certain embodiments, the biological sample may be derived from a cell or tissue, such as blood, serum, plasma, or cerebral spinal fluid, or a cell or tissue of the brain (e.g., cortex or hippocampus), a histological preparation, and the like. In some embodiments, the subject suffers from or is at risk of suffering from Alzheimer's disease or mild cognitive impairment due to Alzheimer's disease. In some embodiments the described methods include detecting the presence or amount of pT217 Tau by contacting the biological sample with the anti-pT217 Tau antibody or anti-pT217 Tau-binding fragment of any one or more of (1) to (21) above.

In certain embodiments, the methods comprise contacting the biological sample with the anti-pT217 Tau antibody or anti-pT217 Tau-binding fragment under conditions permissive for binding of the anti-pT217 Tau antibody or anti-pT217 Tau-binding fragment to pT217 Tau, and detecting whether a complex is formed between anti-pT217 Tau antibody or anti-pT217 Tau-binding fragment and pT217 Tau. In some embodiments of these methods, the anti-pT217 Tau antibody or anti-pT217 Tau-binding fragment is detectably labelled. The methods may be in vitro or in vivo methods. The complex formed between the anti-pT217 Tau antibody or anti-pT217 Tau-binding fragment and pT217 Tau in a test biological sample can be compared to the complex formed in a control biological sample (e.g., a biological sample from a healthy subject). The amount of the complex formed between the anti-pT217 Tau antibody or anti-pT217 Tau-binding fragment and pT217 Tau in a test biological sample can also be quantified and compared to the amount of the complex formed in a control biological sample or to the average amount of the complex known to be formed in healthy subjects. The healthy subject may be an amyloid-negative subject not having amyloid beta accumulation.

In a method for diagnosing Alzheimer's disease or mild cognitive impairment due to Alzheimer's disease, the subject is diagnosed as having Alzheimer's disease or mild cognitive impairment due to Alzheimer's disease when the presence or certain amount of pT217 Tau in the biological sample is detected. In a method for aiding diagnosis of Alzheimer's disease or mild cognitive impairment due to Alzheimer's disease, the subject is diagnosed as having Alzheimer's disease or mild cognitive impairment duc to Alzheimer's disease when the presence or certain amount of pT217 Tau in the biological sample is detected. In a method for evaluating amyloid beta accumulation, the subject is determined as having amyloid beta accumulation when the presence or certain amount of pT217 Tau in the biological sample is detected. In some embodiments, a method for evaluating amyloid beta accumulation in a subject means determining whether the accumulation of amyloid beta in the subject is greater than control. In some embodiments, the control means the accumulation of amyloid beta in healthy subjects. The healthy subject may be an amyloid-negative subject not having amyloid beta accumulation. In some embodiments, the certain amount of pT217 Tau in the test biological sample means that the amount of the complex formed between the anti-pT217 Tau antibody or anti-pT217 Tau-binding fragment and pT217 Tau in a test biological sample is greater than the amount of the complex formed in a control biological sample or the average amount of the complex known to be formed in healthy subjects.

In certain embodiments, any of the anti-pT217 Tau antibodies or anti-pT217 Tau-binding fragments is useful a kit for detecting the presence or amount of pT217 Tau, a kit for diagnosing Alzheimer's disease or mild cognitive impairment due to Alzheimer's disease, a kit for evaluating amyloid beta accumulation. In some embodiments, these kits further comprise a detectable label. In some embodiments, the anti-pT217 Tau antibodies or the pT217 Tau-binding fragments thereof are detectably labelled. In some embodiments, these kits further comprise other reagent, such as substrate buffer, washing solution, stop solution, calibrator (pT217 Tau or its peptide containing phosphorylated Thr217 such as TPSLP(PT)PPTREC (SEQ ID NO: 68)), and indicator reagent, or a package insert with instructions as to how the kits are to be utilized. The package insert may be a paper insert or electronic medium such as a CD, DVD, or floppy disk. In some embodiments, theses kits further comprise at least one microplate (e.g., a 96 well plate). The microplate can be provided with its corresponding plate cover. The microplate can be polystyrene or of any other suitable material. The microplate can have the anti-pT217 Tau antibodies or the pT217 Tau-binding fragments thereof coated inside each well. In some embodiments, theses kits further comprise a software package for analyzing the results.

### EXAMPLES

### Example 1: Generation of Monoclonal Antibodies

To generate anti-pT217 Tau antibodies, first, the peptide sequence TPSLP(PT)PPTREC (SEQ ID NO: 68) was synthesized. This sequence corresponds to residues 212-222 in full length 2N4R Tau and incorporates a phosphorylated threonine (pT) residue to correspond to the phospho-site on Tau at amino acid 217. An additional cysteine residue was added to the peptide at the C-terminus for coupling purposes. For immunization, the peptide antigen was coupled to the Keyhole Limpet Hemocyanin (KLH) carrier protein. The final immunogen was prepared by mixing the peptide antigen-conjugated KLH with Freund's complete adjuvant (1:2 (v/v)). Eight-weck-old, female, B6D2F1/Slc mice were immunized with 0.08 mL per mouse of a 2.5 mg/ml (as carrier protein) immunogen preparation. Approximately 3 weeks following the initial injection, the mice received a booster immunization with peptide antigen-conjugated KLH, now without adjuvant, at 0.05 mL per mouse at the same protein concentration as before.

Once mice with a high antibody titer had been identified, cells were isolated from the medial iliac lymph nodes and fused using polyethylene glycol with mouse myeloma SP2 cells to generate hybridomas. Fused cells were seeded into 96-well plates and cultured in hypoxanthinc-aminopterin-thymidine (HAT) selection medium. Hybridomas were selected based on culture supernatant immunoreactivity in ELISA assays. Briefly, 25 ng of the phospho-peptide antigen TPSLP(PT)PPTREC (SEQ ID NO: 68) conjugated to BSA was used to coat cach well of a 96-well plate (Costar cat. no. 2797) in 10 mM phosphate buffer, pH 7.0 at 37&#8304;C for 1 hour. Plates were blocked in blocking buffer (1% BSA diluted in PBS) at room temperature for 30 minutes. Blocking buffer was removed and various dilutions of hybridoma culture supernatant in the same buffer were added to the plate for 1 hour at room temperature. The plate was washed several times with PBS prior to the addition of an HRP-labelled anti-mouse IgG antibody for 30 minutes at room temperature. Following additional wash steps, antibody binding was detected by addition of 3,3′,5,5′-Tetramethylbenzidine (TMB) substrate. The enzymatic reaction was stopped with an equal volume of 2M H₂SO₄ and the optical density of each well was determined using a plate reader set at wavelength 450 nm. To assess initial phospho-peptide selectivity for each hybridoma culture supernatant an identical ELISA was performed using a non-phosphorylated version of the peptide. Hybridoma cultures were selected based on strong immunoreactivity to the phospho-peptide and low reactivity to the non-phosphorylated peptide. Single cell clones were confirmed by serial dilution and microscopy. The isotype of each antibody of interest was determined using a mouse immunoglobulin isotyping kit (BD Pharmingen). All antibodies were found to be of an IgG2b, kappa isotype. All final hybridomas of interest were cryopreserved in serum-free medium and stored in liquid nitrogen.

Antibody Purification from Hybridomas

Hybridomas were grown in Hybridoma-SFM (Life Technologies) media containing 10% FBS, 1 ng/mL human IL-6 (R&D Systems) and Penicillin/Streptomycin. Cultures were scaled up to 400 mL and supernatant was harvested when cells reached high density. Antibody was captured on Protein A columns equilibrated in 1× SSC (saline sodium citrate) buffer pH7 (Invitrogen), and eluted gently over a gradient to 100% elution buffer: 1× SSC/HCI pH2, and immediately neutralized. Purified antibody was dialyzed in 25 mM sodium phosphate (pH6.5) and 150 mM NaCl, aliquoted, and stored at −80 &#8304;C.

Eight hybridoma clones generating antibodies that recognized the original immunizating phospho-peptide were selected (Table 1).

TABLE 1
Hybridoma clones generated

| Clone name | Mouse Isotype |
|---|---|
| 2D6-2A2 | IgG2b, kappa |
| 2H8-1D5 | IgG2b, kappa |
| 2H8-1G7 | IgG2b, kappa |
| 5F6-4B4 | IgG2b, kappa |
| 5F6-4D1 | IgG2b, kappa |
| 7D11-1C1 | IgG2b, kappa |
| 7D11-1D10 | IgG2b, kappa |
| 7G5-4B8 | IgG2b, kappa |

### Production of the PT3 Comparator Antibody

The PT3 comparator antibody was produced recombinantly. cDNA encoding the variable heavy and variable light domains of the PT3 antibody (SEQ ID NO: 25 and 30 described in WO2018/170351) were cloned in-frame with a leader sequence and the mouse IgG2a kappa backbones in the pCMV6 expression vector (Genscript). Antibody was expressed using the ExpiCHO expression system (ThermoScientific) in accordance with the manufacturer's instructions. PT3 antibody was then purified using the same procedure as described above for the hybridomas.

### Example 2: Inhibition ELISA using Recombinant p-Tau Protein

Preparation of Recombinant p-Tau ELISA Capture Plates

Recombinant full length 2N4R Tau phosphorylated by DYRKIA (Signal Chem) was diluted to a concentration of 0.2 microgram/mL in 100 mM sodium bicarbonate and 33 mM sodium carbonate. One hundred microliters of the diluted solution of recombinant full length 2N4R Tau phosphorylated by DYRKIA was dispensed into a 96-well Nunc Maxisorp plate (ThermoFisher) and incubated at 4° C. overnight. The following day, the phosphorylated Tau was removed, and the plate was washed twice in PBS. Wells were then blocked in 0.25 mL of blocking buffer: 1% BSA (Sigma,) in PBS, and left for 2 hours at room temperature. The blocking buffer was discarded, and the plate was then washed 4 times in PBS containing 0.05% Tween®-20 (Biorad) prior to use.

Preparation of p-Tau Blocking Peptide And Test Antibodies

The p-Tau peptide TPSLP(pT)PPTREC (SEQ ID NO: 68) used as a blocking reagent was identical to that described in Example 1. A ImM master stock of the phosphopeptide was prepared in distilled water. The master stock was then diluted further to a working stock of 3 microM in antibody diluent buffer: 0.1% BSA (Sigma) in PBS. Four microliters of the working stock was then dispensed into a 96-well v-bottom plate prior to addition of the test antibodies.

Purified antibodies were first quantified using a mouse IgG ELISA kit in accordance with the manufacturer's instructions (ThermoFisher). Stock pT217 Tau antibodies of interest were diluted to a concentration of 0.1 microgram/mL (0.67 nM) and scrially diluted 3-fold for a further 9 concentration points in a 96 deep-well plate. For antibodies treated with the blocking peptide (TPSLP(PT)PPTREC; SEQ ID NO: 68), 200 microliters of diluted antibody was added to the v-bottom plate described above and was incubated for 10 minutes at room temperature. The final concentration of blocking peptide in the ELISA assay was approximately 60 nM.

p-Tau ELISA Assay

Antibodies treated with or without the blocking peptide TPSLP(PT)PPTREC (SEQ ID NO: 68) were added to the ELISA capture plate in a final volume of 0.2 mL per well and were incubated for 2 hours at room temperature. Antibody solutions were removed, and the plate was washed with PBS containing 0.05% Tween®-20. Then, 0.1 mL of an anti-mouse-HRP secondary antibody (Jackson) diluted 1:5000 in the same antibody diluent buffer was added to cach well and incubated for 1 hr at room temperature. Secondary antibody was removed, and the plate was washed again prior to addition of 100 microliters TMB substrate (ThermoFisher) to each well. Colour was left to develop for 10 minutes at room temperature in the dark before stopping the reaction by further addition of 100 microliters stop solution (ThermoFisher). Plates were read by measuring absorbance at 450 nm using a PheraStar (BMG Labtech) plate reader.

### Data Analysis

Background absorbance (wells not containing antibody) was subtracted and antibody concentration curves were generated using GraphPad Prism version 7.02 (GraphPad Software). A single data point comparison chart selecting 25 pM of antibody was also generated in excel (Microsoft® 365).

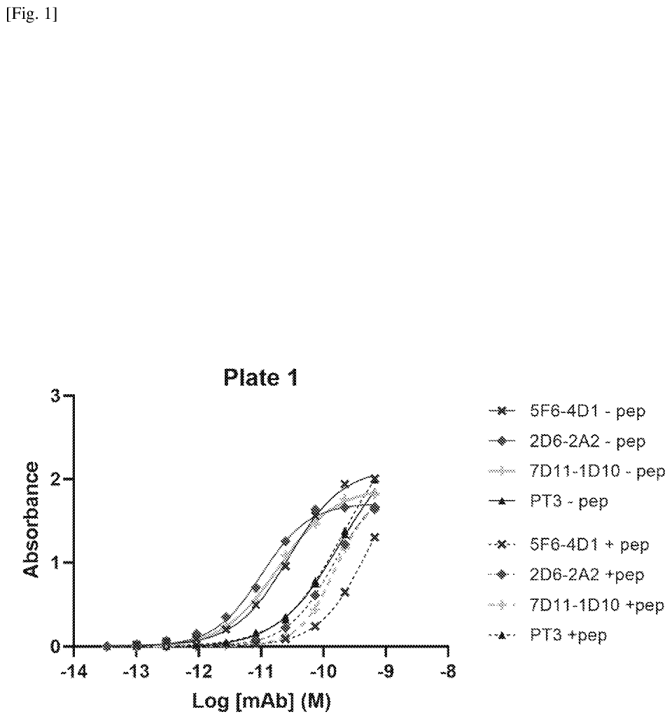

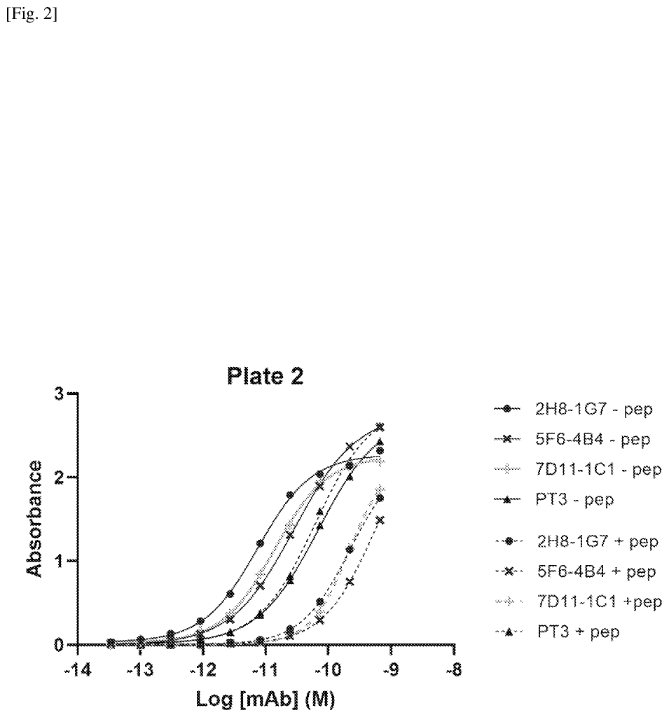

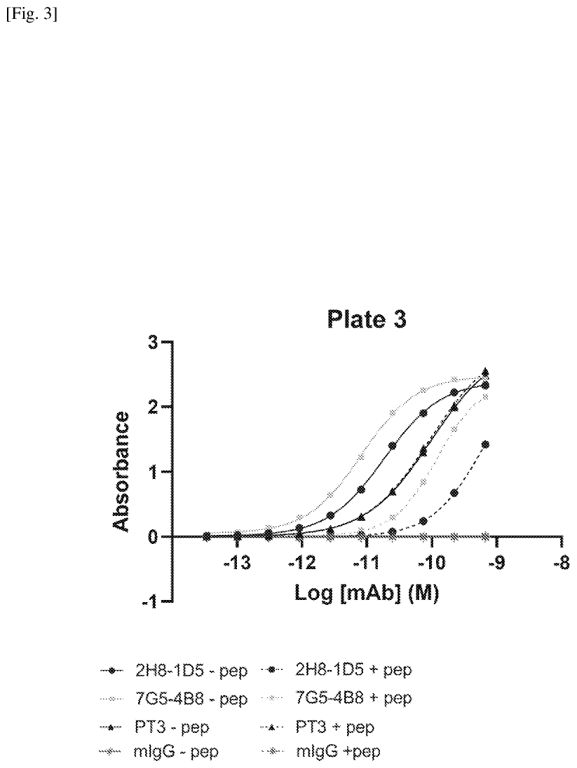

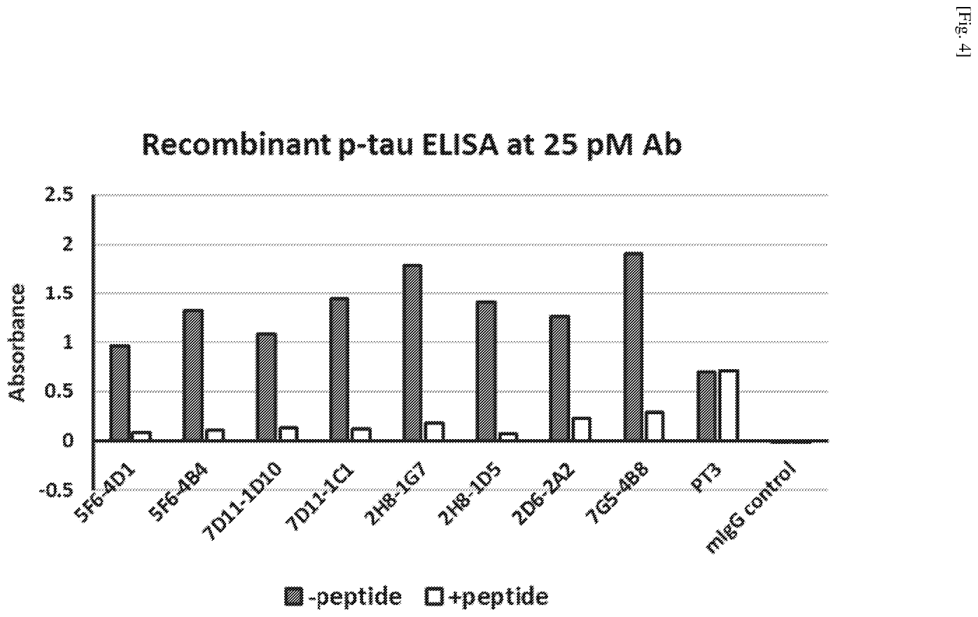

The results are shown in FIGS. 1 to 4. All test antibodies showed strong immunoreactivity for p-Tau in this ELISA assay. In addition, antibodies from the hybridomas showed substantially less reactivity when the blocking peptide was added to the assay. This contrasts with the PT3 comparator antibody where selectivity for pT217 Tau could not be shown.

### Example 3: Inhibition ELISA using Alzheimer's Disease Brain Lysate

### Alzheimer's Disease Brain Lysate Preparation

Human brain tissue provided by Queen Square Brain Bank, London was crushed to a powder in liquid nitrogen and weighed. Powdered tissue was then homogenised using a teflon-glass dounce homogeniser in PBS containing 1× HALT™ protease and phosphatase inhibitor cocktail (ThermoScientific). Crude material was centrifuged at 5,250 g for 20 minutes at 4° C. and the supernatant retained as whole homogenate, which was aliquoted, snap frozen at stored at −80° C. until further use.

In this experiment, stored aliquots of human Braak stage VI Alzheimer's Disease (AD) brain whole homogenates were used. Frozen aliquots were thawed and centrifuged at 21,000 g for 30 minutes at 4° C. and the supernatant was retained for the ELISA assay. Clarified lysate was then diluted to a final 50 microgram/mL protein concentration in antibody diluent (0.1% BSA (Sigma) in PBS) before use.

### Preparation of ELISA Capture Plates

Full length human anti-Tau antibody 7G6-HCzu25/7G6-LCzu15 (described in WO2019/077500) recognizing the Tau MTBR domain was diluted to a concentration of 2 microgram/mL in 100 mM sodium bicarbonate and 33 mM sodium carbonate. 100 microliters of the human antibody solution was then dispensed into a 96-well Nunc Maxisorp plate (ThermoFisher) and incubated at 4° C. overnight. The following day, the capture antibody solution was removed from the plate and the plate was washed twice in PBS. 250 microliters per well of blocking buffer

(1% BSA (Sigma) in PBS) was added to each well of the plate and left for 2 hours at room temperature. The blocking buffer was then discarded, and the plate washed a further 4 times in wash buffer (PBS containing 0.05% tween®-20 (Biorad)) prior to use in the ELISA assay.

Preparation of p-Tau Blocking Peptide and Test Antibodies

Preparation of the blocking peptide and test antibodies was performed as described in Example 2.

### ELISA Assay

100 microliters per well of the clarified Alzheimer's disease brain lysate was added to the Tau capture plate coated with human anti-Tau antibody and left for 2 hours at room temperature. The lysate was then aspirated, and the plate washed 4 times in the same wash buffer (PBS containing 0.05% tween®-20 (Biorad)) as described above. The remainder of the ELISA assay and data analysis was performed exactly as described in Example 2.

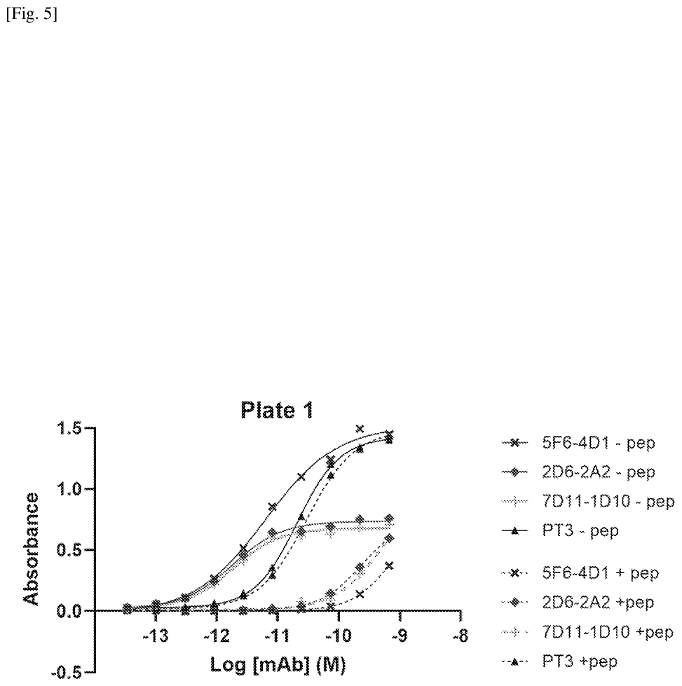

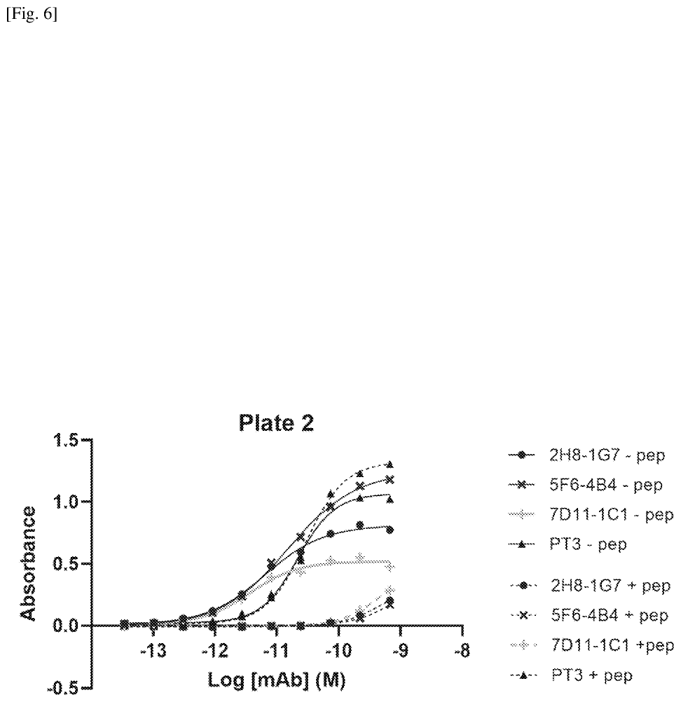

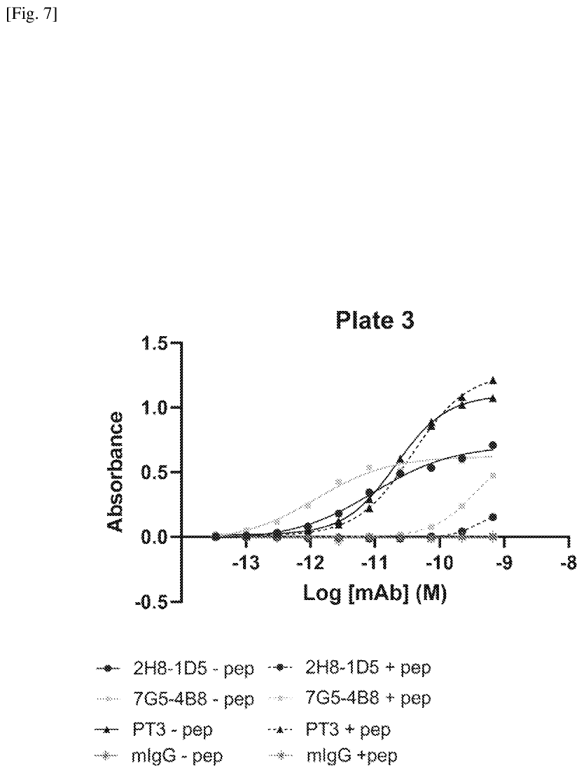

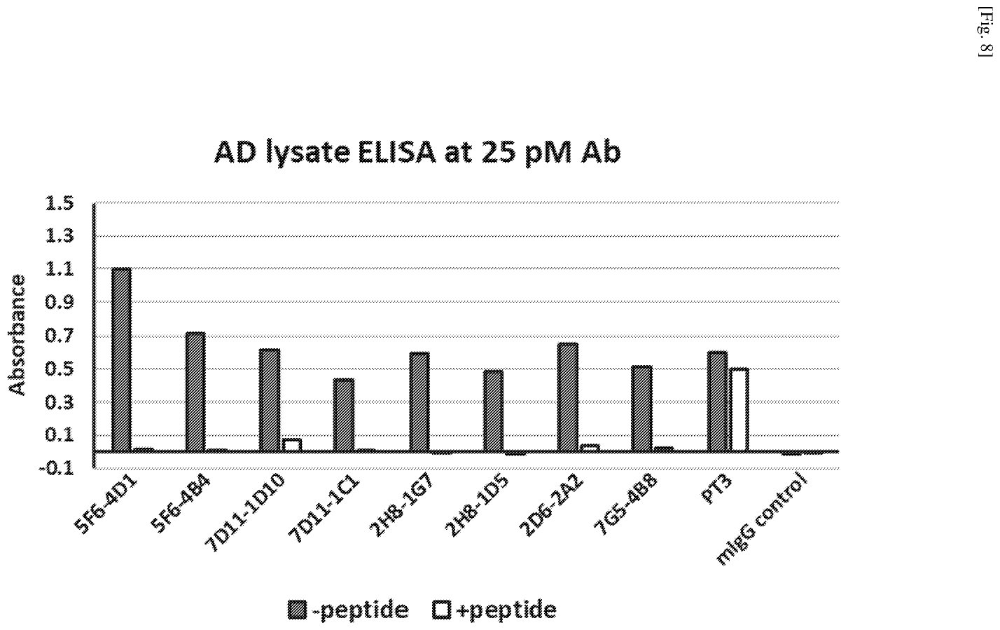

The results are shown in FIGS. 5 to 8. All antibodies tested showed immunoreactivity to the immuno-captured Tau from AD lysates. The antibodies generated from the hybridomas in Example I also showed a high level of selectivity for pT217 Tau as shown by the decrease in reactivity following pre-incubation with the blocking phosphopeptide TPSLP(PT)PPTREC (SEQ ID NO: 68). This contrasts with the PT3 antibody that showed strong immunoreactivity but little selectivity in that immunoreactivity was not greatly affected following incubation with the blocking peptide TPSLP(PT)PPTREC (SEQ ID NO: 68).

Example 4: Western Blot Analysis of Purified Test pT217 Tau Antibodies

### Sample Preparation

Stored frozen aliquots of human brain whole homogenate samples (as described in Example 3) for control brain (Braak stage I), and Alzheimer's Disease brain (Braak stage VI) were used for this experiment. Whole homogenates were centrifuged at 21,000 g for 30 minutes at 4° C. The supernatant was retained and used for western blot analysis. To generate the denatured lysates, 500 microliters of the clarified supernatant was diluted to a protein concentration of 1 mg/mL in LDS sample buffer (LICOR) containing 1× sample reducing agent (Invitrogen). In the case of recombinant Tau and p-Tau proteins, a concentrated stock solution was diluted to 5 microgram/mL in LDS sample buffer. All samples were heated to 100° C. for 5 minutes to denature protein.

### Western Blotting

To detect Tau proteins, 10 micrograms of each brain lysate and 50 ng of each recombinant protein were resolved on 4-12% Bis-Tris Novex gels (Invitrogen) and transferred to Hybond nitrocellulose membranes (GE healthcare). Blots were blocked for 1 hour at room temperature in blocking buffer: a 1:1 mixture of Odyssey blocking buffer (LI-COR) and Tris-buffered saline containing 0.1% Tween®-20 (TBS-T). After blocking, blots were probed with the test antibodies: 2H8-1G7, 5F6-4B4, 5F6-4D1, 7D11-1C1, 2H8-1D5, 7G5-4B8, 7D11-1D10, 2D6-2A2, and comparator antibodies: PT3, and pT217 Tau rabbit polyclonal antibody (ThermoScientific catalogue no. 44-744), at 0.5 microgram/mL in blocking buffer and incubated overnight at 4° C. An additional murine control antibody, 7G6, was also included at the same concentration. 7G6 recognises all forms of Tau containing the MTBR domain (WO2019/077500). Blots were washed three times in TBS-T for 15 minutes cach wash before incubation with an appropriate IRDye 700RD goat anti-mouse or anti-rabbit secondary antibody (LI-COR) diluted at 1:5000 in blocking buffer for 1 hour at room temperature. Secondary antibodies were removed, and blots washed a further 4 times in TBS-T and then once in TBS without Tween®-20. Blots were then scanned, and fluorescent images acquired using an Odyssey LI-COR CLx scanner using Image Studio software (LI-COR).

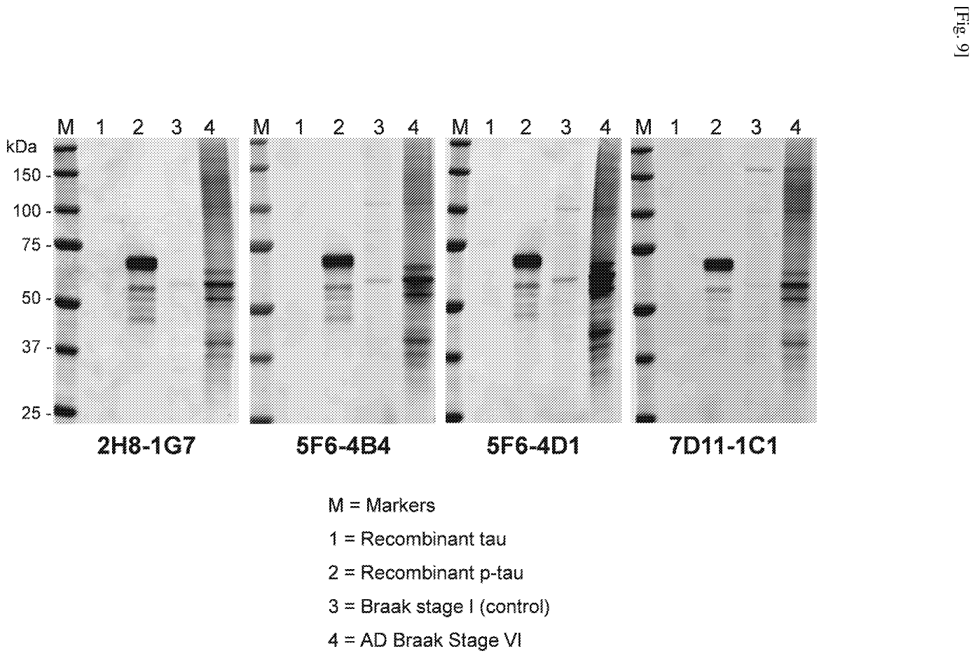

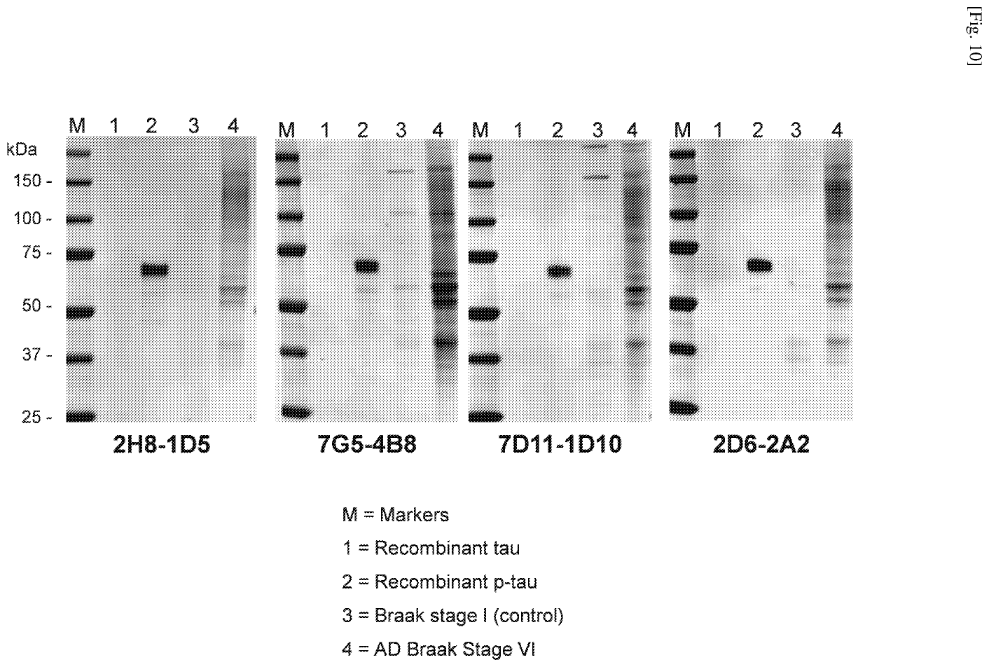

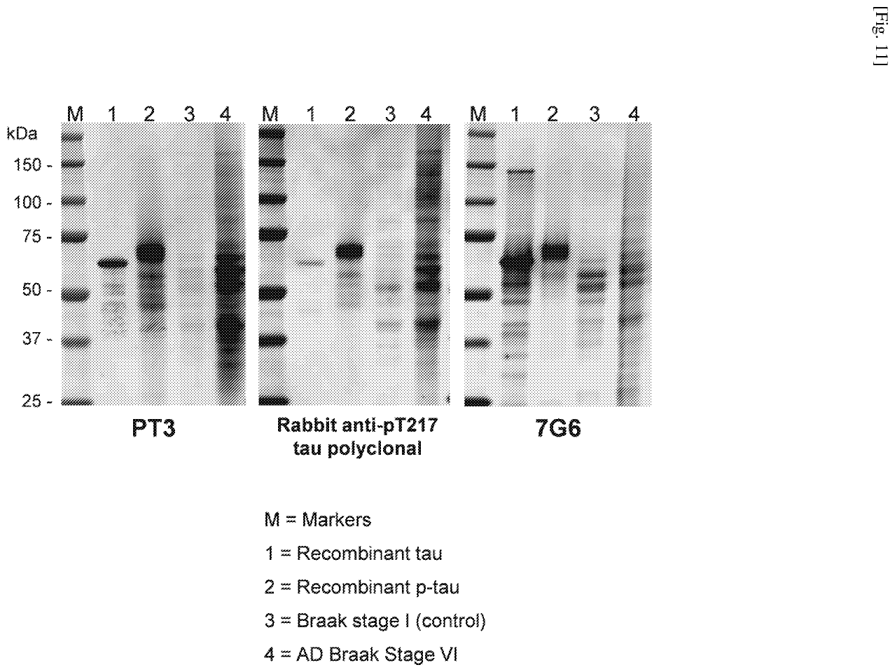

Results are shown in FIGS. 9 to 11. All of the murine test antibodies in Example 1 bound recombinant p-Tau protein but did not display immunoreactivity for the non-phosphorylated form of the protein. In contrast, both PT3 and the control rabbit polyclonal antibody displayed immunoreactivity to the non-phosphorylated Tau. These data indicate that the monoclonal antibodies in Example 1 have superior selectivity for Tau specifically phosphorylated at residue threonine 217 when compared to the comparator antibodies tested herein. All antibodies tested showed greater immunoreactivity for Tau in the extracts of the AD Braak stage VI compared with Tau from extracts of non-AD control brain. The multiple Tau bands observed likely reflect a hetcrogeneity of different truncated and post-translationally modified Tau species observed in human brains (Roberts et al., “Pre-clinical characterisation of E2814, a high-affinity antibody targeting the microtubule-binding repeat domain of tau for passive immunotherapy in Alzheimer's disease” Acta Neuropath Comm. 2020 8(13)). However, stronger immunoreactivity and more bands were observed in the extracts of the control brain when blots were probed with PT3 and the rabbit polyclonal comparator antibodies compared to the murine test antibodies described in Example 1. This again suggests the murine test antibodies in Example 1 have superior selectivity for Tau phosphorylated at residue threonine 217 as observed in AD brains.

### Example 5: Hybridoma Sequencing

Antibody sequencing using a whole transcriptome shotgun sequencing (RNA-Seq) approach for each of the hybridoma clones was performed by Fusion Antibodies P.L.C. (Belfast, Northern Ireland). Briefly, cach hybridoma clone was expanded sufficiently to provide frozen cell pellets containing 1×10⁷ cells in each pellet. RNA was extracted from each pellet and a barcoded cDNA library was generated by RT-PCR using random hexamers. Next Generation Sequencing was then conducted on an

Illumina HiSeq sequencer. All data was mined to identify viable antibody sequences. The variable heavy and variable light domains were identified separately. The Complementarity Determining Regions (CDRs) were determined by the Kabat numbering system. Sequences containing stop codons and known aberrant antibody genes sometimes present in hybridomas were removed from the analysis. From the 8 original hybridomas, 5 unique antibody sequences were identified (Table 2). Clones 5F6-4D1 and 5F6-4B4 had the same antibody sequence. Clones 7D11-1D10 and 7D11-1C1 had the same antibody sequence. Likewise, 2H8-1G7 and 2H8-1D5 were also identical in sequence.

TABLE 2
Antibody sequences of hybridomas

| | | SEQ ID | |
|---|---|---|---|
| Clone Name | | NO | Amino Acid Sequence |
| 2D6-2A2 | Heavy Chain | 1 | DYGMH |
| | CDR1 | | |
| | Heavy Chain | 2 | YMSSGSSTIYYVDTVKG |
| | CDR2 | | |
| | Heavy Chain | 3 | RLSI |
| | CDR3 | | |
| | Heavy Chain | 4 | EVQLVESGGGLVKPGGSLKLSCAASGF |
| | Variable Domain | | TLSDYGMHWVRQAPEKGLEWIAYMSS |
| | | | GSSTIYYVDTVKGRFTISRDNAKNTLFL |
| | | | QMTSLRSEDTAMYYCARRLSIWGTGTT |
| | | | VTVSS |
| | Heavy Chain | 5 | EVQLVESGGGLVKPGGSLKLSCAASGF |
| | | | TLSDYGMHWVRQAPEKGLEWIAYMSS |
| | | | GSSTIYYVDTVKGRFTISRDNAKNTLFL |
| | | | QMTSLRSEDTAMYYCARRLSIWGTGTT |
| | | | VTVSSAKTTPPSVYPLAPGCGDTTGSS |
| | | | VTLGCLVKGYFPESVTVTWNSGSLSSS |
| | | | VHTFPALLQSGLYTMSSSVTVPSSTWPS |
| | | | QTVTCSVAHPASSTTVDKKLEPSGPISTI |
| | | | NPCPPCKECHKCPAPNLEGGPSVFIFPP |
| | | | NIKDVLMISLTPKVTCVVVDVSEDDPD |
| | | | VRISWFVNNVEVHTAQTQTHREDYNST |
| | | | IRVVSALPIQHIQDWMSGKEFKCKVNN |
| | | | KDLPSPIERTISKIKGLVRAPQVYILPPPA |
| | | | EQLSRKDVSLTCLVVGFNPGDISVEWTS |
| | | | NGHTEENYKDTAPVLDSDGSYFIYSKL |
| | | | DIKTSKWEKTDSFSCNVRHEGLKNYYL |
| | | | KKTISRSPFK |
| | Light Chain | 6 | SASSSVSSMY |
| | CDR1 | | |
| | Light Chain | 7 | DTSNLAS |
| | CDR2 | | |
| | Light Chain | 8 | QQWSSYPLT |
| | CDR3 | | |
| | Light Chain | 9 | QIVLTQSPAIMSASPGEKVTMTCSASSS |
| | Variable Domain | | VSSMYWYHQKPGSSPRLLIYDTSNLAS |
| | | | GVPVRFSGSGSGTSYSLTISRMEAEDAA |
| | | | TYYCQQWSSYPLTFGAGTKLELK |
| | Light Chain | 10 | QIVLTQSPAIMSASPGEKVTMTCSASSS |
| | | | VSSMYWYHQKPGSSPRLLIYDTSNLAS |
| | | | GVPVRFSGSGSGTSYSLTISRMEAEDAA |
| | | | TYYCQQWSSYPLTFGAGTKLELKRAD |
| | | | AAPTVSIFPPSSEQLTSGGASVVCFLNN |
| | | | FYPKDINVKWKIDGSERQNGVLNSWT |
| | | | DQDSKDSTYSMSSTLTLTKDEYERHNS |
| | | | YTCEATHKISTSPIVKSENRNEC |
| 2H8-1G7 | Heavy Chain | 1 | DYGMH |
| (2H8-1D5) | CDR1 | | |
| | Heavy Chain | 11 | YISSGSSTIYYVDTVKG |
| | CDR2 | | |
| | Heavy Chain | 3 | RLSI |
| | CDR3 | | |
| | Heavy Chain | 12 | EVQLVESGGGLVKPGGSLKLSCAASGF |
| | Variable Domain | | TVSDYGMHWVRQAPEKGLEWIAYISS |
| | | | GSSTIYYVDTVKGRFTISRDNAKNTLFL |
| | | | QMTSLRSEDTAMYYCARRLSIWGTGTT |
| | | | VTVSS |
| | Heavy Chain | 13 | EVQLVESGGGLVKPGGSLKLSCAASGF |
| | | | TVSDYGMHWVRQAPEKGLEWIAYISS |
| | | | GSSTIYYVDTVKGRFTISRDNAKNTLFL |
| | | | QMTSLRSEDTAMYYCARRLSIWGTGTT |
| | | | VTVSSAKTTPPSVYPLAPGCGDTTGSS |
| | | | VTLGCLVKGYFPESVTVTWNSGSLSSS |
| | | | VHTFPALLQSGLYTMSSSVTVPSSTWPS |
| | | | QTVTCSVAHPASSTTVDKKLEPSGPISTI |
| | | | NPCPPCKECHKCPAPNLEGGPSVFIFPP |
| | | | NIKDVLMISLTPKVTCVVVDVSEDDPD |
| | | | VRISWFVNNVEVHTAQTQTHREDYNST |
| | | | IRVVSALPIQHQDWMSGKEFKCKVNN |
| | | | KDLPSPIERTISKIKGLVRAPQVYILPPPA |
| | | | EQLSRKDVSLTCLVVGFNPGDISVEWTS |
| | | | NGHTEENYKDTAPVLDSDGSYFIYSKL |
| | | | DIKTSKWEKTDSFSCNVRHEGLKNYYL |
| | | | KKTISRSPGK |
| | Light Chain | 6 | SASSSVSSMY |
| | CDR1 | | |
| | Light Chain | 7 | DTSNLAS |
| | CDR2 | | |
| | Light Chain | 14 | LQWSSYPLT |
| | CDR3 | | |
| | Light Chain | 15 | QIVLTQSPAIMSACPGEKVTMTCSASSS |
| | Variable Domain | | VSSMYWYHQKPGSSPRLLIYDTSNLAS |
| | | | GVPVRFSGSGSGTSYSLTISRMEAEDAA |
| | | | TYYCLQWSSYPLTFGAGTKLELK |
| | Light Chain | 16 | QIVLTQSPAIMSACPGEKVTMTCSASSS |
| | | | VSSMYWYHQKPGSSPRLLIYDISNLAS |
| | | | GVPVRFSGSGSGTSYSLTISRMEAEDAA |
| | | | TYYCLQWSSYPLTFGAGTKLELKRADA |
| | | | APTVSIFPPSSEQLTSGGASVVCFLNNF |
| | | | YPKDINVKWKIDGSERQNGVLNSWTD |
| | | | QDSKDSTYSMSSTLTLTKDEYERHNSY |
| | | | TCEATHKTSTSPIVKSFNRNEC |
| 5F6-4D1 | Heavy Chain | 17 | DYGIH |
| (5F6-4B4) | CDR1 | | |
| | Heavy Chain | 18 | YISSGSNTIYYIDTVKG |
| | CDR2 | | |
| | Heavy Chain | 19 | RMAY |
| | CDR3 | | |
| | Heavy Chain | 20 | EVQLVESGGGLVKPGGSLKLSCVASGF |
| | Variable Domain | | SVSDYGIHWYRQAPEKGLEWVAYISSG |
| | | | SNTIYYIDTVKGRFTISRDNAKNTLFLQ |
| | | | MTSLRSEDTAMYYCARRMAYWGQGT |
| | | | LVTVSA |
| | Heavy Chain | 21 | EVQLVESGGGLVKPGGSLKLSCVASGF |
| | | | SVSDYGIHWVRQAPEKGLEWVAYISSG |
| | | | SNTIYYIDTVKGRFTISRDNAKNTLFLQ |
| | | | MTSLRSEDTAMYYCARRMAYWGQGT |
| | | | LVTVSAAKTTPPSVYPLAPGCGDTTGSS |
| | | | VTLGCLVKGYFPESYVTVTWNSGSLSSS |
| | | | VHTFPALLQSGLYTMSSSVTVPSSTWPS |
| | | | QTVTCSVAHPASSTTVDKKLEPSGPISTI |
| | | | NPCPPCKECHKCPAPNLEGGPSVFIFPP |
| | | | NIKDVLMISLTPKVTCVVVDVSEDDPD |
| | | | VRISWFVNNVEVHTAQTQTHREDYNST |
| | | | IRVVSALPIQHQDWMSGKEFKCKVNN |
| | | | KDLPSPIERTISKIKGLVRAPQVYILPPPA |
| | | | EQLSRKDVSLTCLVVGFNPGDISVEWTS |
| | | | NGHTEENYKDTAPVLDSDGSYFIYSKL |
| | | | DIKTSKWEKTDSFSCNVRHEGLKNYYL |
| | | | KKTISRSPGK |
| | Light Chain | 6 | SASSSVSSMY |
| | CDR1 | | |
| | Light Chain | 22 | ATFNLAS |
| | CDR2 | | |
| | Light Chain | 8 | QQWSSYPLT |
| | CDR3 | | |
| | Light Chain | 23 | QIVLTQSPAIMSTSPGEKVTMTCSASSS |
| | Variable Domain | | VSSMYWYQQKPGSSPRLLIYATFNLAS |
| | | | GVPVRFSGSGSGTSYSLTISRMEAEDAA |
| | | | TYYCQQWSSYPLTFGAGTKLEVK |
| | Light Chain | 24 | QIVLTQSPAIMSTSPGEKVTMTCSASSS |
| | | | VSSMYWYQQKPGSSPRLLIYATFNLAS |
| | | | GVPVRFSGSGSGTSYSLTISRMEAEDAA |
| | | | TYYCQQWSSYPLTFGAGTKLEVKRAD |
| | | | AAPTVSIFPPSSEQLTSGGASVVCFLNN |
| | | | FYPKDINVKWKIDGSERQNGVLNSWT |
| | | | DQDSKDSTYSMSSTLTLTKDEYERHNS |
| | | | YTCEATHKTSTSPIVKSFNRNEC |
| 7D11-1D10 | Heavy Chain | 1 | DYGMH |
| (7D11-1C1) | CDR1 | | |
| | Heavy Chain | 25 | YISSGDSTIYYADTVKG |
| | CDR2 | | |
| | Heavy Chain | 19 | RMAY |
| | CDR3 | | |
| | Heavy Chain | 26 | EVQLVESGGGLVKPGGSLKLSCAASGF |
| | Variable Domain | | TLSDYGMHWVRQAPEKGLEWFAYISS |
| | | | GDSTIYYADTVKGRFTISRDNAKNTLFL |
| | | | QMTSLRSEDTAMYYCARRMAYWGQG |
| | | | TLVTVSA |
| | Heavy Chain | 27 | EVQLVESGGGLVKPGGSLKLSCAASGF |
| | | | TLSDYGMHWVRQAPEKGLEWFAYISS |
| | | | GDSTIYYADTVKGRFTISRDNAKNTLFL |
| | | | QMTSLRSEDTAMYYCARRMAYWGQG |
| | | | TLVTVSAAKTTPPSVYPLAPGCGDTTG |
| | | | SSVTLGCLVKGYFPESVTVTWNSGSLS |
| | | | SSVHTFPALLQSGLYTMSSSVTVPSSTW |
| | | | PSQTVTCSVAHPASSTTVDKKLEPSGPIS |
| | | | TINPCPPCKECHKCPAPNLEGGPSVFIFP |
| | | | PNIKDVLMISLIPKVTCVVVDVSEDDP |
| | | | DVRISWFVNNVEVHTAQTQTHREDYN |
| | | | STIRVVSALPIQHQDWMSGKEFKCKVN |
| | | | NKDLPSPIERTISKIKGLVRAPQVYILPPP |
| | | | AEQLSRKDVSLTCLVVGFNPGDISVEW |
| | | | TSNGHTEENYKDTAPVLDSDGSYFIYS |
| | | | KLDIKTSKWEKTDSFSCNVRHEGLKNY |
| | | | YLKKTISRSPGK |
| | Light Chain | 28 | SASSSVTYMY |
| | CDR1 | | |
| | Light Chain | 29 | ATSNLAS |
| | CDR2 | | |
| | Light Chain | 8 | QQWSSYPLT |
| | CDR3 | | |
| | Light Chain | 30 | QIVLTQSPAIMSASPGEKVTMTCSASSS |
| | Variable Domain | | VTYMYWYQQKPGSSPRLLIYATSNLAS |
| | | | GVPVRFSGSGSGTSYSLTISRMEAEDAA |
| | | | TYYCQQWSSYPLTFGAGTKLELK |
| | Light Chain | 31 | QIVLTQSPAIMSASPGEKVTMTCSASSS |
| | | | VTYMYWYQQKPGSSPRLLIYATSNLAS |
| | | | GVPVRFSGSGSGTSYSLTISRMEAEDAA |
| | | | TYYCQQWSSYPLTFGAGTKLELKRAD |
| | | | AAPTVSIFPPSSEQLTSGGASVVCFLNN |
| | | | FYPKDINVKWKIDGSERQNGVLNSWT |
| | | | DQDSKDSTYSMSSTLTLTKDEYERHNS |
| | | | YTCEATHKTSTSPIVKSFNRNEC |
| 7G5-4B8 | Heavy Chain | 1 | DYGMH |
| | CDR1 | | |
| | Heavy Chain | 32 | YISSGSSTTYYADTVKG |
| | CDR2 | | |
| | Heavy Chain | 19 | RMAY |
| | CDR3 | | |
| | Heavy Chain | 33 | EVQLVESGGGLVKPGGSLKLSCSASGF |
| | Variable Domain | | TISDYGMHWVRQAPEKGLEWFAYISSG |
| | | | SSTIYYADTVKGRFTISRDNAKNTLFLQ |
| | | | MTSLRSEDTAMYYCARRMAYWGQGT |
| | | | LVTVSA |
| | Heavy Chain | 34 | EVQLVESGGGLVKPGGSLKLSCSASGF |
| | | | TISDYGMHWVRQAPEKGLEWFAYISSG |
| | | | SSTIYYADTVKGRFTISRDNAKNTLFLQ |
| | | | MTSLRSEDTAMYYCARRMAYWGQGT |
| | | | LVTVSAAKTTPPSVYPLAPGCGDTTGSS |
| | | | VTLGCLVKGYFPESVTVTWNSGSLSSS |
| | | | VHTFPALLQSGLYTMSSSVTVPSSTWPS |
| | | | QTVTCSVAHPASSTTVDKKLEPSGPISTI |
| | | | NPCPPCKECHKCPAPNLEGGPSVFIFPP |
| | | | NIKDVLMISLTPKVTCVVVDVSEDDPD |
| | | | VRISWFVNNVEVHTAQTQTHREDYNST |
| | | | IRVVSALPIQHQDWMSGKEFKCKVNN |
| | | | KDLPSPIERTISKIKGLVRAPQVYILPPPA |
| | | | EQLSRKDVSLTCLVVGFNPGDISVEWTS |
| | | | NGHTEENYKDTAPVLDSDGSYFIYSKL |
| | | | DIKTSKWEKTDSFSCNVRHEGLKNYYL |
| | | | KKTISRSPGK |
| | Light Chain | 6 | SASSSVSSMY |
| | CDR1 | | |
| | Light Chain | 29 | ATSNLAS |
| | CDR2 | | |
| | Light Chain | 8 | QQWSSYPLT |
| | CDR3 | | |
| | Light Chain | 35 | QIVLTQSPAIMSASPGEKVTMTCSASSS |
| | Variable Domain | | VSSMYWYQQKPGSSPRLLIYATSNLAS |
| | | | GVPVRFSGSGSGTSYSTISRMEAEDAA |
| | | | TYYCQQWSSYPLTFGAGTKLELK |
| | Light Chain | | QIVLTQSPAIMSASPGEKVTMTCSASSS |
| | | | VSSMYWYQQKPGSSPRLLIYATSNLAS |
| | | | GVPVRFSGSGSGTSYSLTISRMEAEDAA |
| | | | TYYCQQWSSYPLTFGAGTKLELKRAD |
| | | | AAPTVSIFPPSSEQLTSGGASVVCFLNN |
| | | | FYPKDINVKWKIDGSERQNGVLNSWT |
| | | | DQDSKDSTYSMSSTLTLTKDEYERHNS |
| | | | YTCEATHKTSTSPIVKSFNRNEC |

### Example 6: Expression and Purification of Recombinant Anti-pT217 Tau Antibodies

Genes encoding heavy chains of antibody 5F6-4D1 (SEQ ID NO: 34). 7G5-4B8 (SEQ ID NO: 21) and 2H8-1G7 (SEQ ID NO: 13), and genes encoding light chains of antibody 5F6-4D1 (SEQ ID NO: 24), 7G5-4B8 (SEQ ID NO: 36) and 2H8-1G7 (SEQ ID NO: 16) were fused by gene encoding N-terminal signal sequence of heavy chain (amino acid: MEWSWVFLFFLSVTTGVHS; SEQ ID NO: 69; nucleotide: ATG-GAGTGGAGCTGGGTGTTCCTGTTCTTTCTGAGCGTGACCACAGGCGTGCAC TCC; SEQ ID NO: 70) and gene encoding N-terminal signal sequence of light chain (amino acid: MSVPTQVLGLLLLWLTDARC; SEQ ID NO: 71; nucleotide: ATGTC-CGTGCCTACACAGGTGCTGGGACTGCTGCTGCTGTGGCTGACCGACGCCAG ATGC; SEQ ID NO: 72), respectively. Fusion genes were synthesized and cloned into pcDNA3.4 expression vector (Thermo Fisher Scientific). An abnormal cysteine residue at Kabat numbering position 14 on the light chain of 2H8-1G7 was substituted with serine residue, to give antibody 2H8-1G7(KC14S) with a mutated light chain (SEQ ID NO: 64). Heavy chain constant regions and light chain constant regions of these antibodies were mouse IgG2b and mouse Ig kappa, respectively. 5F6-4D1, 7G5-4B8, 2H8-1G7 and 2H8-1G7(KC14S) were expressed by transfecting genes to Expi293F cells (Thermo Fisher Scientific), then the antibodies were purified from supernatants by using Mab Select (Cytiva).

TABLE 3
Nucleotide sequences of 2H8-1G7, 5F6-4D1 and 7G5-4B8

| | | SEQ ID | |
|---|---|---|---|
| Clone Name | | NO | Nucleotide Sequence |
| 2H8-1G7 | Heavy Chain | 37 | GACTACGGCATGCAC |
| | CDR1 | | |
| | Heavy Chain | 38 | TATATCAGCTCCGGCTCTAGCACCATC |
| | CDR2 | | TACTATGTGGACACAGTGAAGGGC |
| | Heavy Chain | 39 | AGACTGTCCATC |
| | CDR3 | | |
| | Heavy Chain | 40 | GAGGTGCAGCTGGTGGAGTCCGGAG |
| | Variable Domain | | GAGGACTGGTGAAGCCAGGAGGCTC |
| | | | CCTGAAGCTGTCTTGCGCCGCCAGCG |
| | | | GCTTTACCGTGAGCGACTACGGCATG |
| | | | CACTGGGTGAGGCAGGCACCTGAGA |
| | | | AGGGACTGGAGTGGATCGCCTATATC |
| | | | AGCTCCGGCTCTAGCACCATCTACTAT |
| | | | GTGGACACAGTGAAGGGCAGGTTCA |
| | | | CCATCTCCCGCGATAACGCCAAGAAT |
| | | | ACACTGTTTCTGCAGATGACCAGCCT |
| | | | GCGCTCCGAGGACACAGCCATGTACT |
| | | | ATTGTGCACGGAGACTGTCCATCTGG |
| | | | GGAACCGGAACCACAGTGACAGTGT |
| | | | CCTCT |
| | Heavy Chain | 41 | GAGGTGCAGCTGGTGGAGTCCGGAG |
| | | | GAGGACTGGTGAAGCCAGGAGGCTC |
| | | | CCTGAAGCTGTCTTGCGCCGCCAGCG |
| | | | GCTTTACCGTGAGCGACTACGGCATG |
| | | | CACTGGGTGAGGCAGGCACCTGAGA |
| | | | AGGGACTGGAGTGGATCGCCTATATC |
| | | | AGCTCCGGCTCTAGCACCATCTACTAT |
| | | | GTGGACACAGTGAAGGGCAGGTTCA |
| | | | CCATCTCCCGCGATAACGCCAAGAAT |
| | | | ACACTGTTTCTGCAGATGACCAGCCT |
| | | | GCGCTCCGAGGACACAGCCATGTACT |
| | | | ATTGTGCACGGAGACTGTCCATCTGG |
| | | | GGAACCGGAACCACAGTGACAGTGT |
| | | | CCTCTGCCAAGACCACACCACCTAGC |
| | | | GTGTACCCTCTGGCACCAGGATGCGG |
| | | | CGATACCACAGGAAGCTCCGTGACCC |
| | | | TGGGCTGTCTGGTGAAGGGCTATTTC |
| | | | CCAGAGAGCGTGACCGTGACATGGA |
| | | | ACAGCGGCTCCCTGTCTAGCTCCGTG |
| | | | CACACATTTCCCGCCCTGCTGCAGTC |
| | | | CGGACTGTACACCATGTCTAGCTCCGT |
| | | | GACCGTGCCATCTAGCACATGGCCCT |
| | | | CCCAGACCGTGACATGCTCTGTGGCC |
| | | | CACCCTGCCTCCTCTACCACAGTGGA |
| | | | CAAGAAGCTGGAGCCCTCCGGCCCTA |
| | | | TCTCTACCATCAATCCCTGCCCACCCT |
| | | | GTAAGGAGTGCCACAAGTGTCCAGCA |
| | | | CCTAACCTGGAGGGAGGACCTAGCGT |
| | | | GTTCATCTTTCCTCCAAATATCAAGGA |
| | | | TGTGCTGATGATCAGCCTGACCCCTAA |
| | | | GGTGACATGCGTGGTGGTGGACGTGA |
| | | | GCGAGGACGATCCAGATGTGCGGATC |
| | | | TCCTGGTTCGTGAACAATGTGGAGGT |
| | | | GCACACAGCCCAGACCCAGACACAC |
| | | | CGGGAGGACTACAACAGCACCATCAG |
| | | | AGTGGTGTCCGCCCTGCCAATCCAGC |
| | | | ACCAGGACTGGATGTCCGGCAAGGA |
| | | | GTTTAAGTGCAAGGTGAACAATAAGG |
| | | | ATCTGCCATCTCCCATCGAGAGGACC |
| | | | ATCAGCAAGATCAAGGGACTGGTGCG |
| | | | CGCACCACAGGTGTACATCCTGCCAG |
| | | | CTCCAGCAGAGCAGCTGAGCAGAAA |
| | | | GGACGTGTCCCTGACATGTCTGGTGG |
| | | | TGGGCTTCAATCCTGGCGATATCTCTG |
| | | | TGGGAGTGGACCAGCAACGGCCACAC |
| | | | AGAGGAGAATTATAAGGATACCGCCC |
| | | | CAGTGCTGGACTCTGATGGCAGCTAC |
| | | | TTCATCTATTCCAAGCTGGACATCAAG |
| | | | ACATCTAAGTGGGAGAAGACCGATTC |
| | | | TTTTAGCTGCAACGTGCGGCACGAGG |
| | | | GCCTGAAGAATTACTATCTGAAGAAG |
| | | | ACCATCTCCAGATCTCCCGGCAAG |
| | Light Chain | 42 | AGCGCCAGCTCCTCTGTGAGCTCCAT |
| | CDR1 | | GTAC |
| | Light Chain | 43 | GATACATCTAACCTGGCAAGC |
| | CDR2 | | |
| | Light Chain | 44 | CTGCAGTGGTCCTCTTACCCCCTGAC |
| | CDR3 | | A |
| | Light Chain | 45 | CAGATCGTGCTGACCCAGAGCCCTGC |
| | Variable Domain | | AATCATGTCCGCCTGCCCAGGAGAGA |
| | | | AGGTGACCATGACATGTAGCGCCAGC |
| | | | TCCTCTGTGAGCTCCATGTACTGGTAT |
| | | | CACCAGAAGCCAGGCTCTAGCCCCCG |
| | | | GCTGCTGATCTACGATACATCTAACCT |
| | | | GGCAAGCGGAGTGCCCGTGAGATTCT |
| | | | CCGGCTCTGGCAGCGGCACATCCTATT |
| | | | CTCTGACCATCTCCAGGATGGAGGCA |
| | | | GAGGACGCAGCAACCTACTATTGCCT |
| | | | GCAGTGGTCCTCTTACCCCCTGACATT |
| | | | CGGCCCCGGCACCAAGCTGGAGCTG |
| | | | AAG |
| | Light Chain | 46 | CAGATCGTGCTGACCCAGAGCCCTGC |
| | | | AATCATGTCCGCCTGCCCAGGAGAGA |
| | | | AGGTGACCATGACATGTAGCGCCAGC |
| | | | TCCTCTGTGAGCTCCATGTACTGGTAT |
| | | | CACCAGAAGCCAGGCTCTAGCCCCCG |
| | | | GCTGCTGATCTACGATACATCTAACCT |
| | | | GGCAAGCGGAGTGCCCGTGAGATTCT |
| | | | CCGGCTCTGGCAGCGGCACATCCTATT |
| | | | CTCTGACCATCTCCAGGATGGAGGCA |
| | | | GAGGACGCAGCAACCTACTATTGCCT |
| | | | GCAGTGGTCCTCTTACCCCCTGACATT |
| | | | CGGCGCCGGCACCAAGCTGGAGCTG |
| | | | AAGAGAGCCGATGCCGCCCCTACAGT |
| | | | GAGCATCTTTCCCCCTAGCTCCGAGC |
| | | | AGCTGACCTCCGGAGGAGCATCTGTG |
| | | | GTGTGCTTCCTGAACAACTTCTACCC |
| | | | AAAGGACATCAACGTGAAGTGGAAG |
| | | | ATCGATGGCTCTGAGAGGCAGAACGG |
| | | | CGTGCTGAATAGCTGGACAGACCAGG |
| | | | ATTCTAAGGACAGCACCTATTCCATGT |
| | | | CTAGCACCCTGACACTGACCAAGGAT |
| | | | GAGTACGAGCGCCACAATTCCTATAC |
| | | | ATGCGAGGCCACCCACAAGACAAGC |
| | | | ACCTCCCCCATCGTGAAGTCTTTTAAC |
| | | | CGGAATGAGTGT |
| 5F6-4D1 | Heavy Chain | 47 | GACTACGGCATCCAC |
| | CDR1 | | |
| | Heavy Chain | 48 | TATATCAGCTCCGGCAGCAACACCATC |
| | CDR2 | | TACTATATCGACAGAGTGAAGGGC |
| | Heavy Chain | 49 | AGAATGGCATAC |
| | CDR3 | | |
| | Heavy Chain | 50 | GAGGTGCAGCTGGTGGAGTCTGGAG |
| | Variable Domain | | GAGGACTGGTGAAGCCAGGAGGCTC |
| | | | TCTGAAGCTGAGCTGCGTGGCCTCCG |
| | | | GCTTTTCTGTGAGCGACTACGGCATCC |
| | | | ACTGGGTGAGGCAGGCACCTGAGAA |
| | | | GGGACTGGAGTGGGTGGCCTATATCA |
| | | | GCTCCGGCAGCAACACCATCTACTATA |
| | | | TCGACACAGTGAAGGGCAGGTTCACC |
| | | | ATCTCCCGCGATAACGCCAAGAATAC |
| | | | ACTGTTTCTGCAGATGACCTCCCTGC |
| | | | GCTCTGAGGACACAGCCATGTACTATT |
| | | | GTGCAAGGAGAATGGCATACTGGGGA |
| | | | CAGGGCACCCTGGTGACAGTGTCCGC |
| | | | C |
| | Heavy Chain | 51 | GAGGTCCAGCTGGTGGAGTCTGGAG |
| | | | GAGGACTGGTGAAGCCAGGAGGCTC |
| | | | TCTGAAGCTGAGCTGCGTGGCCTCCG |
| | | | GCTTTTCTGTGAGCGACTACGGCATCC |
| | | | ACTGGGTGAGGCAGGCACCTGAGAA |
| | | | GGGACTGGAGTGGGTGGCCTATATCA |
| | | | GCTCCGGCAGCAACACCATCTACTATA |
| | | | TCGACACAGTGAAGGGCAGGTTCACC |
| | | | ATCTCCCGCGATAACGCCAAGAATAC |
| | | | ACTGTTTCTGCAGATGACCTCCCTGC |
| | | | GCTCTGAGGACACAGCCATGTACTATT |
| | | | GTGCAAGGAGAATGGCATACTGGGGA |
| | | | CAGGGCACCCTGGTGACAGTGTCCGC |
| | | | CGCCAAGACCACACCACCTAGCGTGT |
| | | | ACCCTCTGGCACCAGGATGCGGCGAT |
| | | | ACCACAGGATCTAGCTGACCCTGGG |
| | | | CTGTCTGGTGAAGGGCTACTTCCCAG |
| | | | AGAGCGTGACCGTGACATGGAACTCC |
| | | | GGCTCTCTGTCCTCTAGCGTGCACAC |
| | | | ATTTCCCGCCCTGCTGCAGTCCGGCCT |
| | | | GTATACCATGTCCTCTAGCGTGACCGT |
| | | | GCCATCCTCTACATGGCCCTCTCAGAC |
| | | | CGTGACATGCAGCGTGGCCCACCCTG |
| | | | CCAGCTCCACCACAGTGGACAAGAA |
| | | | GCTGGAGCCCTCTGGCCCTATCAGCA |
| | | | CCATCAATCCCTGCCCACCCTGTAAGG |
| | | | AGTGCCACAAGTGTCCAGCACCTAAC |
| | | | CTGGAGGGAGGACCTAGCGTGTTCAT |
| | | | CTTTCCTCCAAATATCAAGGATGTGCT |
| | | | GATGATCTCCCTGACCCCTAAGGTGA |
| | | | CATGCGTGGTGGTGGACGTGAGCGAG |
| | | | GACGATCCAGATGTGCGGATCTCCTG |
| | | | GTTCGTGAACAATGTGGAGGTGCACA |
| | | | CAGCCCAGACCCAGACACACCGGGA |
| | | | GGACTACAACTCCACCATCAGAGTGG |
| | | | TGTCTGCCCTGCCAATCCAGCACCAG |
| | | | GACTGGATGAGCGGCAAGGAGTTTAA |
| | | | GTGCAAGGTGAACAATAAGGATCTGC |
| | | | CAAGCCCCATCGAGAGGACCATCTCC |
| | | | AAGATCAAGGGACTGGTGCGCGCACC |
| | | | ACAGGTGTACATCCTGCCACCTCCAG |
| | | | CAGAGCAGCTGTCCAGAAAGGACGT |
| | | | GTCTCTGACATGTCTGGTGGTGGGCTT |
| | | | CAATCCTGGCGATATCAGCGTGGAGT |
| | | | GGACCTCCAACGGCCACACAGAGGA |
| | | | GAATTATAAGGATACCGCCCCAGTGCT |
| | | | GGACAGCGATGGCTCCTACTTCATCTA |
| | | | TTCTAAGCTGGACATCAAGACAAGCA |
| | | | AGTGGGAGAAGACCGATAGCTTTTCC |
| | | | TGCAACGTGCGGCACGAGGGCCTGA |
| | | | AGAATTACTATCTGAAGAAGACCATCT |
| | | | CTAGAAGCCCCGGCAAG |
| | Light Chain | 42 | AGCGCCAGCTCCTCTGTGAGCTCCAT |
| | CDR1 | | GTAC |
| | Light Chain | 52 | GCCACATTCAACCTGGCATCC |
| | CDR2 | | |
| | Light Chain | 53 | CAGCAGTGGTCCTCTTACCCCCTGAC |
| | CDR3 | | A |
| | Light Chain | 54 | CAGATCGTGCTGACACAGTCTCCTGC |
| | Variable Domain | | CATCATGTCCACCTCTCCAGGCGAGA |
| | | | AGGTGACCATGACATGTAGCGCCAGC |
| | | | TCCTCTGTGAGCTCCATGTACTGGTAT |
| | | | CAGCAGAAGCCAGGCTCTAGCCCCCG |
| | | | GCTGCTGATCTACGCCACATTCAACCT |
| | | | GGCATCCGGAGTGCCCGTGCGGTTCA |
| | | | GCGGCTCCGGCTCTGGCACAAGCTAT |
| | | | TCCCTGACCATCAGCAGGATGGAGGC |
| | | | AGAGGACGCAGCAACCTACTATTGCC |
| | | | AGCAGTGGTCCTCTTACCCCCTGACAT |
| | | | TCGGCGCCGGCACCAAGCTGGAGGT |
| | | | GAAG |
| | Light Chain | 55 | CAGATCGTGCTGACACAGTCTCCTGC |
| | | | CATCATGTCCACCTCTCCAGGCGAGA |
| | | | AGGTGACCATGACATGTAGCGCCAGC |
| | | | TCCTCTGTGAGCTCCATGTACTGGTAT |
| | | | CAGCAGAAGCCAGGCTCTAGCCCCCG |
| | | | GCTGCTGATCTACGCCACATTCAACCT |
| | | | GGCATCCGGAGTGCCCGTGCGGTTCA |
| | | | GCGGCTCCGGCTCTGGCACAAGCTAT |
| | | | TCCCTGACCATCAGCAGGATGGAGGG |
| | | | AGAGGACGCAGCAACCTACTATTGCC |
| | | | AGCAGTGGTCCTCTTACCCCCTGACAT |
| | | | TCGGCGCCGGCACCAAGCTGGAGGT |
| | | | GAAGAGAGCCGATGCCGCCCCTACAG |
| | | | TGAGCATCTTTCCCCCTAGCTCCGAGC |
| | | | AGCTGACCTCCGGAGGAGCATCTGTG |
| | | | GTGTGCTTCCTGAACAACTTCTACCC |
| | | | AAAGGACATCAACGTGAAGTGGAAG |
| | | | ATCGATGGCAGCGAGAGGCAGAACG |
| | | | GCGTGCTGAACAGCTGGACAGACCA |
| | | | GGATTCCAAGGACTCTACCTATAGCAT |
| | | | GTCTAGCACCCTGACACTGACCAAGG |
| | | | ATGAGTACGAGCGCCACAATTCCTATA |
| | | | CATGCGAGGCCACCCACAAGACATCT |
| | | | ACCAGCCCCATCGTGAAGTCTTTCAA |
| | | | CCGGAATGAGTGT |
| 7G5-4B8 | Heavy Chain | 37 | GACTACGGCATGCAC |
| | CDR1 | | |
| | Heavy Chain | 56 | TATATCAGCTCCGGCTCTAGCACCATC |
| | CDR2 | | TACTATGCCGACACAGTGAAGGGC |
| | Heavy Chain | 49 | AGAATGGCATAC |
| | CDR3 | | |
| | Heavy Chain | 57 | GAGGTGCAGCTGGTGGAGTCTGGAG |
| | Variable Domain | | GAGGACTGGTGAAGCCAGGAGGCTC |
| | | | TCTGAAGCTGTCCTGCTCTGCCAGCG |
| | | | GCTTCACCATCTCCGACTACGGCATGC |
| | | | ACTGGGTGAGGCAGGCACCTGAGAA |
| | | | GGGACTGGAGTGGTTTGCCTATATCA |
| | | | GCTCCGGCTCTAGCACCATCTACTATG |
| | | | CCGACACAGTGAAGGGCAGGTTCAC |
| | | | CATCAGCCGCGATAACGCCAAGAATA |
| | | | CACTGTTTCTGCAGATGACCTCCCTGC |
| | | | GCTCTGAGGACACAGCCATGTACTATT |
| | | | GTGCAAGGAGATGGCATACTGGGGA |
| | | | CAGGGCACCCTGGTGACAGTGTCCGC |
| | | | C |
| | Heavy Chain | 58 | GAGGTGCAGCTGGTGGAGTCTGGAG |
| | | | GAGGACTGGTGAAGCCAGGAGGCTC |
| | | | TCTGAAGCTGTCCTGCTCTGCCAGCG |
| | | | GCTTCACCATCTCCGACTACGGCATGC |
| | | | ACTGGGTGAGGCAGGCACCTGAGAA |
| | | | GGGACTGGAGTGGTTTGCCTATATCA |
| | | | GCTCCGGCTCTAGCACCATCCTACTATG |
| | | | CCGACACAGTGAAGGGCAGGTTCAC |
| | | | CATCAGCCGCGATAACGCCAAGAATA |
| | | | CACTGTTTCTGCAGATGACCTCCCTGC |
| | | | GCTCTGAGGACACAGCCATGTACTATT |
| | | | GTGCAAGGAGAATGGCATACTGGGGA |
| | | | CAGGGCACCCTGGTGACAGTGTCCGC |
| | | | CGCCAAGACCACACCACCTAGCGTGT |
| | | | ACCCTCTGGCACCAGGATGCGGCGAT |
| | | | ACCACAGGATCCTCTGTGACCCTGGG |
| | | | CTGTCTGGTGAAGGGCTACTTCCCAG |
| | | | AGAGCGTGACCGTGACATGGAACTCC |
| | | | GGCTCTCTGAGCTCCTCTGTGCACAC |
| | | | ATTTCCCGCCCTGCTGCAGTCCGGCCT |
| | | | GTATACCATGAGCTCCTCTGTGACCGT |
| | | | GCCAAGCTCCACATGGCCCTCTCAGA |
| | | | CCGTGACATGCAGCGTGGCCCACCCT |
| | | | GCCTCTAGCACCACAGTGGACAAGAA |
| | | | GCTGGAGCCCTCTGGCCCTATCAGCA |
| | | | CCATCAATCCCTGCCCACCCTGTAAGG |
| | | | AGTGCCACAAGTGTCCAGCACCTAAC |
| | | | CTGGAGGGAGGACCTAGCGTGTTCAT |
| | | | CTTTCCTCCAAATATCAAGGATGTGCT |
| | | | GATGATCTCCCTGACCCCTAAGGTGA |
| | | | CATGCGTGGTGGTGGACGTGAGCGAG |
| | | | GACGATCCAGATGTGCGGATCTCCTG |
| | | | GTTCGTGAACAATGTGGAGGTGCACA |
| | | | CAGCCCAGACCCAGACACACCGGGA |
| | | | GGACTACAACTCCACCATCAGAGTGG |
| | | | TGTCTGCCCTGCCAATCCAGCACCAG |
| | | | GACTGGATGAGCGGCAAGGAGTTTAA |
| | | | GTGCAAGGTGAACAATAAGGATCTGC |
| | | | CAAGCCCATCCGAGAGGACCATCTCC |
| | | | AAGATCAAGGGACTGGTGCGCGCACC |
| | | | ACAGGTGTACATCCTGCCACCTCCAG |
| | | | CAGAGCACTGTCCAGAAAGGACGT |
| | | | GTCTCTGACATGTCTGGTGGTGGGCTT |
| | | | TAATCCTGGCGATATCAGCGTGGAGTG |
| | | | GACCTCCAACGGCCACACAGAGGAG |
| | | | AATTATAAGGATACCGCCCAGTGCTG |
| | | | GACAGCGATGGCTCCTACTTCATCTAT |
| | | | TCTAAGCTGGACATCAAGACAAGCAA |
| | | | GTGGGAGAAGACCGATAGCTTTTCCT |
| | | | GCAACGTGCGGCACGAGGGCCTGAA |
| | | | GAATTACTATCTGAAGAAGACCATCTC |
| | | | TAGAAGCCCCGGCAAG |
| | Heavy Chain | 59 | TCTGCCAGCTCCTCTGTGAGCTCCATG |
| | CDR1 | | TAC |
| | Heavy Chain | 60 | GCCACATCCAACCTGGCCTCT |
| | CDR2 | | |
| | Heavy Chain | 53 | CAGCAGTGGTCCTCTTACCCCCTGAC |
| | CDR3 | | A |
| | Light Chain | 61 | CAGATCGTGCTGACCCAGTCCCCTGC |
| | Variable Domain | | CATCATGTCCGCCTCTCCAGGCGAGA |
| | | | AGGTGACCATGACATGTTCTGCCAGC |
| | | | TCCTCTGTGAGCTCCATGTACTGGTAT |
| | | | CAGCAGAAGCCAGGCTCTAGCCCCCG |
| | | | GCTGCTGATCTACGCCACATCCAACCT |
| | | | GGCCTCTGGAGTGCCCGTGAGATTCA |
| | | | GCGGATCCGGATCTGGCACAAGCTAT |
| | | | TCCCTGACCATCTCCAGGATGGAGGC |
| | | | AGAGGACGCAGCAACCTACTATTGCC |
| | | | AGCAGTGGTCCTCTTACCCCCTGACAT |
| | | | TCGGCGCGGCACCAAGCTGGAGCTG |
| | | | AAG |
| | Light Chain | 62 | CAGATCGTGCTGACCCAGTCCCCTGC |
| | | | CATCATGTCCGCCTCTCCAGGCGAGA |
| | | | AGGTGACCATGACATGTTCTGCCAGC |
| | | | TCCTCTGTGAGCTCCATGTACTGGTAT |
| | | | CAGCAGAAGCCAGGCTCTAGCCCCCG |
| | | | GCTGCTGATCTACGCCACATCCAACCT |
| | | | GGCCTCTGGAGTGCCCGTGAGATTCA |
| | | | GCGGATCCGGATCTGGCACAAGCTAT |
| | | | TCCCTGACCATCTCCAGGATGGAGGC |
| | | | AGAGGACGCAGCAACCTACTATTGCC |
| | | | AGCAGTGGTCCTCTTACCCCCTGACAT |
| | | | TCGGCGCCGGCACCAAGCTGGAGCTG |
| | | | AAGAGAGCCGATGCCGCCCCTACAGT |
| | | | GAGCATCTTTCCCCCTAGCTCCGAGC |
| | | | AGCTGACCAGCGGAGGAGCATCCGTG |
| | | | GTGTGCTTCCTGAACAACTTCTACCC |
| | | | AAAGGACATCAACGTGAAGTGGAAG |
| | | | ATCGATGGCTCTGAGAGGCAGAACGG |
| | | | CGTGCTGAATAGCTGGACAGACCAGG |
| | | | ATTCCAAGGACTCTACCTATAGCATGT |
| | | | CTAGCACCCTGACACTGACCAAGGAT |
| | | | GAGTACGAGCGCCACAATTCCTATAC |
| | | | ATGCGAGGCCACCCACAAGACATCTA |
| | | | CCAGCCCCATCGTGAAGAGCTTTAAC |
| | | | CGGAATGAGTGT |

TABLE 4
Sequences of 2H8-1G7(KC14S)

| | | SEQ ID | |
|---|---|---|---|
| Clone Name | | NO | Amino Acid Sequence |
| 2H8- | Heavy Chain | 1 | DYGMH |
| 1G7(KC14S) | CDR1 | | |
| | Heavy Chain | 11 | YISSGSSTIYYVDTVKG |
| | CDR2 | | |
| | Heavy Chain | 3 | RLSI |
| | CDR3 | | |
| | Heavy Chain | 12 | EVQLVESGGGLVKPGGSLKLSCAASGF |
| | Variable Domain | | TVSDYOGMHWVRQAPEKGLEWIAYISS |
| | | | GSSTIYYVDTVKGRFTISRDNAKNTLFL |
| | | | QMTSLRSEDTAMYYCARRLSIWGTGTT |
| | | | VTVSS |
| | Heavy Chain | 13 | EVQLVESGGGLVKPGGSLKLSCAASGF |
| | | | TVSDYGMHWVRQAPEKGLEWIAYISS |
| | | | GSSTIYYYDTVKGRFTISRDNAKNTLFL |
| | | | QMTSLRSEDTAMYYCARRLSIWGTGTT |
| | | | VTVSSAKTTPPSVYPLAPGCGDTTGSS |
| | | | VTLGCLVKGYFPESVTVTWNSGSLSSS |
| | | | VHTFPALLQSGLYTMSSSVTVPSSTWPS |
| | | | QTVTCSVAHPASSTTVDKKLEPSGPISTI |
| | | | NPCPPCKECHKCPAPNLEGGPSVFIFPP |
| | | | NIKDVLMISLTPKVTCVVVDVSEDDPD |
| | | | VRISWFVNNVEVHTAQTQTHREDYNST |
| | | | IRVVSALPIQHQDWMSGKEFKCKVNN |
| | | | KDLPSPIERTISKIKGLVRAPQVYILPPPA |
| | | | EQLSRKDVSLTCLVVGFNPGDISVEWTS |
| | | | NGHTEENYKDTAPVLDSDGSYFIYSKL |
| | | | DIKTSKWEKTDSFSCNVRHEGLKNYYL |
| | | | KKTISRSPGK |
| | Light Chain | 6 | SASSSVSSMY |
| | CDR1 | | |
| | Light Chain | 7 | DTSNLAS |
| | CDR2 | | |
| | Light Chain | 14 | LQWSSYPLT |
| | CDR3 | | |
| | Light Chain | 63 | QIVLTQSPAIMSASPGEKVTMTCSASSS |
| | Variable Domain | | VSSMYWYHQKPGSSPRLLIYDTSNLAS |
| | | | GVPVRFSGSGSGTSYSLTISRMEAEDAA |
| | | | TYYCLQWSSYPLTFGAGTKLELK |
| | Light Chain | 64 | QIVLTQSPAIMSASPGEKVTMTCSASSS |
| | | | VSSMYWYHQKPGSSPRLLIYDTSNLAS |
| | | | GYPVRFSGSGSGTSYSLTISRMEAEDAA |
| | | | TYYCLQWSSYPLTFGAGTKLELKRADA |
| | | | APTVSIFPPSSEQLTSGGASVVCFLNNF |
| | | | YPKDINVKWKIDGSERQNGVLNSWTD |
| | | | QDSKDSTYSMSSTLTLTKDEYERHNSY |
| | | | TCEATHKTSTSPIVKSFNRNEC |
| | | SEQ ID | |
| | | NO | Nucleotide Sequence |
| | Heavy Chain | 37 | GACTACGGCATGCAC |
| | CDR1 | | |
| | Heavy Chain | 38 | TATATCAGCTCCGGCTCTAGCACCATC |
| | CDR2 | | TACTATGTGGACACAGTGAAGGGC |
| | Heavy Chain | 39 | AGACTGTCCATC |
| | CDR3 | | |
| | Heavy Chain | 40 | GAGGTGCAGCTGGTGGAGTCCGGAG |
| | Variable Domain | | GAGGACTGGTGAAGCCAGGAGGCTC |
| | | | CCTGAAGCTGTCTTGCGCCGCCAGCG |
| | | | GCTTTACCGTGAGCGACTACGGCATG |
| | | | CACTGGGTGAGGCAGGCACCTGAGA |
| | | | AGGGACTGGAGTGGATCGCCTATATC |
| | | | AGCTCCGGCTCTAGCACCATCTACTAT |
| | | | GTGGACACAGTGAAGGGCAGGTTCA |
| | | | CCATCTCCCGCGATAACGCCAAGAAT |
| | | | ACACTGTTTCTGCAGATGACCAGCCT |
| | | | GCGCTCCGAGGACACAGCCATGTACT |
| | | | ATTGTGCACGGAGACTGTCCATCTGG |
| | | | GGAACCGGAACCACAGTGACAGTGT |
| | | | CCTCT |
| | Heavy Chain | 41 | GAGGTGCAGCTGGTGGAGTCCGGAG |
| | | | GAGGACTGGTGAAGCCAGGAGGCTC |
| | | | CCTGAAGCTGTCTTGCGCOGCCAGCG |
| | | | GCTTTACCGTGAGCGACTACGGCATG |
| | | | CACTGGGTGAGGCAGGCACCTGAGA |
| | | | AGGGACTGGAGTGGATCGCCTATATC |
| | | | AGCTCCGGCTCTAGCACCATCTACTAT |
| | | | GTGGACACAGTGAAGGGCAGGTTCA |
| | | | CCATCTCCCGCGATAACGCCAAGAAT |
| | | | ACACTGTTTCTGCAGATGACCAGCCT |
| | | | GCGCTCCGAGGACACAGCCATGTACT |
| | | | ATTGTGCACGGAGACTGTCCATCTGG |
| | | | GGAACCGGAACCACAGTGACAGTGT |
| | | | CCTCTGCCAAGACCACACCACCTAGG |
| | | | GTGTACCCTCTGGCACCAGGATGCGG |
| | | | CGATACCACAGGAAGCTCCGTGACCC |
| | | | TGGGCTGTCTGGTGAAGGGCTATTTC |
| | | | CCAGAGAGCGTGACCGTGACATGGA |
| | | | ACAGCGGCTCCCTGTCTAGCTCCGTG |
| | | | CACACATTTCCCGCCCTGCTGCAGTC |
| | | | CGGACTGTACACCATGTCTAGCTCCGT |
| | | | GACCGTGCCATCTAGCACATGGCCCT |
| | | | CCCAGACCGTGACATGCTCTGTGGCC |
| | | | CACCCTGCCTCCTCTACCACAGTGGA |
| | | | CAAGAAGCTGGAGCCCTCCGGCCCTA |
| | | | TCTCTACCATCAATCCCTGCCCACCCT |
| | | | GTAAGGAGTGCCACAAGTGTCCAGCA |
| | | | CCTAACCTGGAGGGAGGACCTAGCGT |
| | | | GTTCATCTTTCCTCCAAATATCAAGGA |
| | | | TGTGCTGATGATCAGCCTGACCCCTAA |
| | | | GGTGACATGCGTGGTGGTGGACGTGA |
| | | | GCGAGGACGATCCAGATGTGCGGATC |
| | | | TCCTGGTTCGTGAACAATGTGGAGGT |
| | | | GCACACAGCCCAGACCCAGACACAC |
| | | | CGGGAGGACTACAACAGCACCATCAG |
| | | | AGTGGTGTCCGCCCTGCCAATCCAGC |
| | | | ACCAGGACTGGATGTCCGGCAAGGA |
| | | | GTTTAAGTGCAAGGTGAACAATAAGG |
| | | | ATCTGCCATCTCCCATCGAGAGGACC |
| | | | ATCAGCAAGATCAAGGGACTGGTGCG |
| | | | CGCACCACAGGTGTACATCCTGCCAC |
| | | | CTCCAGCAGAGCAGCTGAGCAGAAA |
| | | | GGACGTGTCCCTGACATGTCTGGTGG |
| | | | TGGGCTTCAATCCTGGCGATATCTCTG |
| | | | TGGAGTGGACCAGCAACGGCCACAC |
| | | | AGAGGAGAATTATAAGGATACCGCCC |
| | | | CAGTGCTGGACTCTGATGGCAGCTAC |
| | | | TTCATCTATTCCAAGCTGGACATCAAG |
| | | | ACATCTAAGTGGGAGAAGACCGATTC |
| | | | TTTTAGCTGCAACGTGCGCACGAGG |
| | | | GCCTGAAGAATTACTATCTGAAGAAG |
| | | | ACCATCTCCAGATCTCCCGGCAAG |
| | Light Chain | 42 | AGCGCCAGCTCCTCTGTGAGCTCCAT |
| | CDR1 | | GTAC |
| | Light Chain | 43 | GATACATCTAACCTGGCAAGC |
| | CDR2 | | |
| | Light Chain | 44 | CTGCAGTGGTCCTCTTACCCCCTGAC |
| | CDR3 | | A |
| | Light Chain | 65 | CAGATCGTGCTGACCCAGAGCCCTGC |
| | Variable Domain | | AATCATGTCCGCCAGCCCAGGAGAGA |
| | | | AGGTGACCATGACATGTAGCGCCAGC |
| | | | TCCTCTGTGAGCTCCATGTACTGGTAT |
| | | | CACCAGAAGCCAGGCTCTAGCCCCCG |
| | | | GCTGCTGATCTACGATACATCTAACCT |
| | | | GGCAAGCGGAGTGCCCGTGAGATTCT |
| | | | CCGGCTCTGGCAGCGGCACATCCTATT |
| | | | CTCTGACCATCTCCAGGATGGAGGCA |
| | | | GAGGACGCAGCAACCTACTATTGCCT |
| | | | CCAGTGGTCCTCTTACCCCCTGACATT |
| | | | CGGCGCCGGCACCAAGCTGGAGCTG |
| | | | AAG |
| | Light Chain | 66 | CAGATCGTGCTGACCCAGAGCCCTGC |
| | | | AATCATGTCCGCCAGCCCAGGAGAGA |
| | | | AGGTGACCATGACATGTAGCGCCAGC |
| | | | TCCTCTGTGAGCTCCATGTACTGGTAT |
| | | | CACCAGAAGCCAGGCTCTAGCCCCCG |
| | | | GCTGCTGATCTACGATACATCTAACCT |
| | | | GGCAAGCGGAGTGCCCGTGAGATTCT |
| | | | CCGGCTCTGGCAGCGGCACATCCTATT |
| | | | CTCTGACCATCTCCAGGATGGAGGCA |
| | | | GAGGACGCAGCAACCTACTATTGCCT |
| | | | GCAGTGGTCCTCTTACCCCCTGACATT |
| | | | CGGCGCCGGCACCAAGCTGGAGCTG |
| | | | AAGAGAGCCGATGCCGCCCCTACAGT |
| | | | GAGCATCTTTCCCCCTAGCTCCGAGC |
| | | | AGCTGACCTCCGGAGGAGCATCTGTG |
| | | | GTGTGCTTCCTGAAGAACTTCTACCC |
| | | | AAAGGACATCAACGTGAAGTGGAAG |
| | | | ATCGATGGCTCTGAGAGGCAGAACGG |
| | | | CGTGCTGAATAGCTGGACAGACCAGG |
| | | | ATTCTAAGGACAGCACCTATTCCATGT |
| | | | CTAGCACCCTGACACTGACCAAGGAT |
| | | | GAGTACGAGCGCCACAATTCCTATAC |
| | | | ATGCGAGGCCACCCACAAGACAAGC |
| | | | ACCTCCCCCATCGTGAAGTCTTTTAAC |
| | | | CGGAATGAGTGT |

### Example 7: Reactivity to pT217 Tau Peptide by Sandwich ELISA

Anti-Tau monoclonal antibody BT2 (Thermo Fisher Scientific) was adjusted with 50 mM Tris/HCl (pH 7.5) to a concentration of 1 microgram/mL, and then injected at 100 microliter/well in a Maxisorp cup (NUNC). The cup was placed in a humid box and coated overnight at 4 degrees Celsius. After the antibody solution was removed by aspiration, a blocking solution (5% skimmed milk (FUJIFILM Wako Pure Chemical), 50 mM Tris/HCI (pH 7.5), 150 mM NaCl, 0.1% sodium azide) was injected at 200 microliter/well, followed by blocking at room temperature for 2 hours or at 4 degrees Celsius for one day or more (hereinafter referred to as “BT2 cup”). The test recombinant antibodies: 5F6-4D1, 7G5-4B8, 2H8-1G7, 2H8-1G7(KC14S) and comparator antibodies: PT3 and anti-pT217 Tau polyclonal antibody (Genscript poly; Cat. No. A00896, Genscript) were labeled with peroxidase using a Peroxidase Labeling Kit-NH2 (DOJINDO MOLECULAR TECHNOLOGIES) in accordance with the instruction manual attached to the kit (hereinafter referred to as “peroxidase-labeled pT217 Tau antibodies”).

The BT2 cup was washed three times with a washing solution (50 mM Tris/HCI (pH 7.5), 150 mM NaCl, 0.01% Tween®-20), and a calibrator long pT217 Tau peptide (KSGDRSGYSSPGSPGTPGSRSRTPSLP(PT)PPTREPKK (SEQ ID NO: 73); P217L) which had been diluted to 10,000 to 13.7 pg/mL by 3-fold manner and 0 pg/mL (blank) with a reaction buffer (5% skimmed milk (FUJIFILM Wako Pure Chemical), 50 mM Tris/HCl (pH 7.5), 150 mM NaCl, 0.2% EDTA-3Na, 0.01% Tween®-20, 4% polyethylene glycol 6000, 0.2% ProClin 150 (SUPELCO, 49376-U, Sigma-Aldrich) was injected at 100 microliter/well and reacted at room temperature for 2 hours. After washing three times with the washing solution, the peroxidase-labeled pT217 Tau antibodies which had been diluted to 100 ng/mL with a reaction buffer was injected at 100 microliter/well and reacted at room temperature for 1 hour. After washing three times with the washing solution, a 3,3′,5,5′-Tetramethylbenzidine (TMB) Liquid Substrate System for ELISA (KPL, SeraCare Life Sciences) was injected at 100 microliter/well and reacted at room temperature for 30 minutes. 0.5 M H₂SO₄ was injected at 100 microliter/well to stop the reaction, and OD (450 to 650 nm) was then measured for each well.

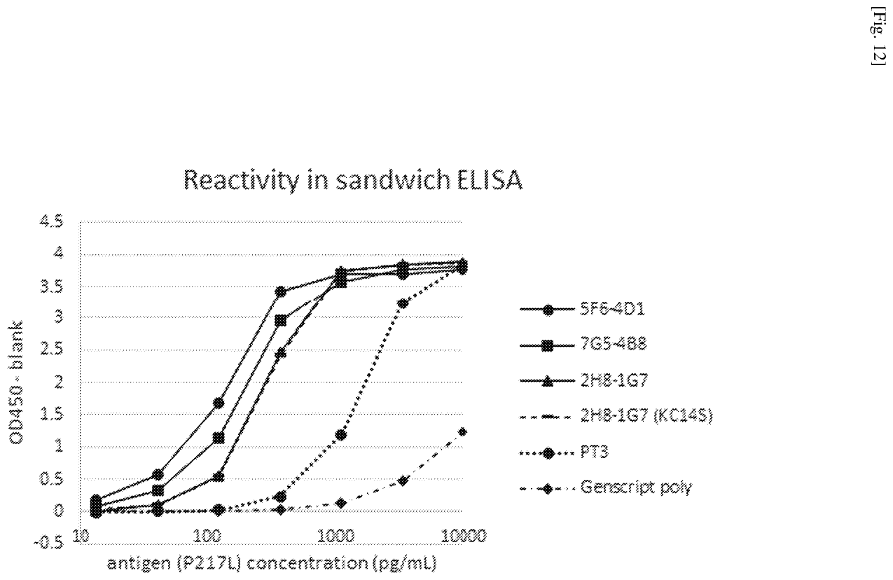

FIG. 12 shows the calibration curves for 5F6-4D1, 7G5-4B8, 2H8-1G7,

2H8-1G7(KC14S), PT3 and commercial pT217 Tau polyclonal antibody (Genscript poly). The sensitivity of sandwich ELISA with 5F6-4D1, 7G5-4B8, 2H8-1G7 and 2H8-1G7(KC14S) was far higher than those with PT3 and Genscript poly. That is, 5F6-4D1, 7G5-4B8, 2H8-1G7, 2H8-1G7(KC14S) have very high reactivity against pT217 Tau sequence on sandwich ELISA.

### Example 8: Selectivity to pT217 Tau Sequence by Sandwich ELISA

Anti-Tau monoclonal antibody BT2 (Thermo Fisher Scientific) was adjusted with 50 mM Tris/HCl (pH 7.5) to a concentration of 1 microgram/mL, and then injected at 100 microliter/well in a Maxisorp cup (NUNC). The cup was placed in a humid box and coated overnight at 4 degrees Celsius. After the antibody solution was removed by aspiration, a blocking solution (5% skimmed milk (FUJIFILM Wako Pure Chemical), 50 mM Tris/HCl (pH 7.5), 150 mM NaCl, 0.1% sodium azide) was injected at 200 microliter/well, followed by blocking at room temperature for 2 hours or at 4 degrees Celsius for one day or more (hereinafter referred to as “BT2 cup”). The test recombinant antibodies: 5F6-4D1, 7G5-4B8, 2H8-1G7, 2H8-1G7(KC14S) and comparator antibodies: PT3 and anti-pT217 Tau polyclonal antibody (Genscript poly; Cat. No. A00896, Genscript) were labeled with peroxidase using a Peroxidase Labeling Kit-NH2 (DOJINDO MOLECULAR TECHNOLOGIES) in accordance with the instruction manual attached to the kit (hereinafter referred to as “peroxidase-labeled pT217 Tau antibodies”).

The BT2 cup was washed three times with a washing solution (50 mM Tris/HCl (pH 7.5), 150 mM NaCl, 0.01% Tween®-20), and a long pT217 Tau peptide (KSGDRSGYSSPGSPGTPGSRSRTPSLP(PT)PPTREPKK (SEQ ID NO: 73); P217L) which had been diluted to 400 to 6.25 pg/mL for 5F6-4D1, 7G5-4B8, 2H8-1G7 and 2H8-1G7(KC14S), 1,000 to 15.6 pg/mL for PT3 and 10,000 to 156 for Genscript poly cach by 2-fold manner and 0 pg/mL (blank), and non-phosphorylated Tau (Tau-441 (2N4R), rPeptide LLC) which had been diluted to 80,000 to 1,250 pg/mL for 5F6-4D1, 7G5-4B8, 2H8-1G7 and 2H8-1G7(KC14S), 200,000 to 3,125 pg/mL for PT3 and 2,000,000 to 31,250 for Genscript poly cach by 2-fold manner and 0 pg/mL (blank) with a reaction buffer (5% skimmed milk (FUJIFILM Wako Pure Chemical), 50 mM Tris/HCl (pH 7.5), 150 mM NaCl, 0.2% EDTA-3Na, 0.01% Tween®-20, 4% polyethylene glycol 6000, 0.2% ProClin 150 (SUPELCO, 49376-U, Sigma-Aldrich) was injected at 100 microliter/well and reacted at room temperature for 2 hours. After washing three times with the washing solution, the peroxidase-labeled pT217 Tau antibodies which had been diluted to 33 ng/ml for 5F6-4D1, 40 ng/mL for 7G5-4B8, 80 ng/ml for 2H8-1G7 and 2H8-1G7(KC14S), 133 ng/mL for PT3 and Genscript poly cach with a reaction buffer was injected at 100 microliter/well and reacted at room temperature for 1 hour. After washing three times with the washing solution, a 3,3′,5,5′-Tetramethylbenzidine (TMB) Liquid Substrate System for ELISA (KPL, SeraCare Life Sciences) was injected at 100 microliter/well and reacted at room temperature for 30 minutes. 0.5 M H₂SO₄ was injected at 100 microliter/well to stop the reaction, and OD (450 to 650 nm) was then measured for each well.

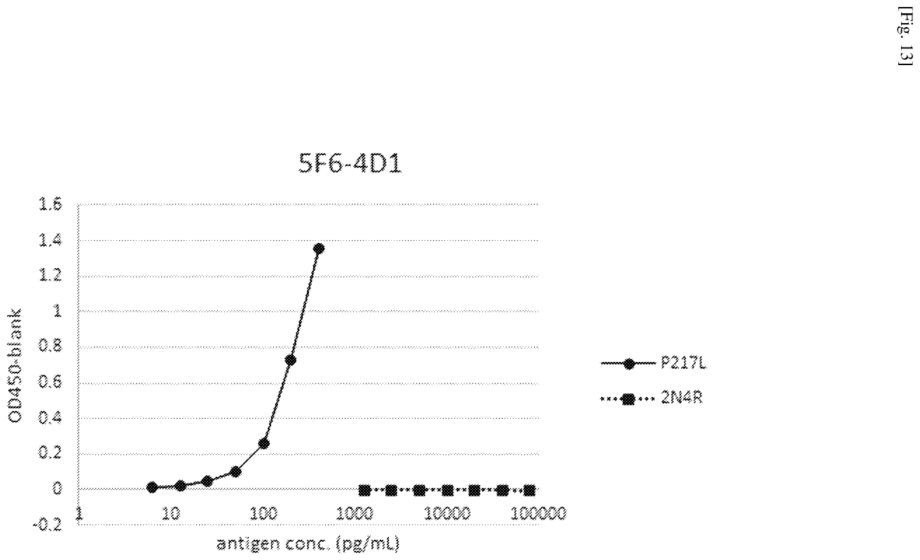

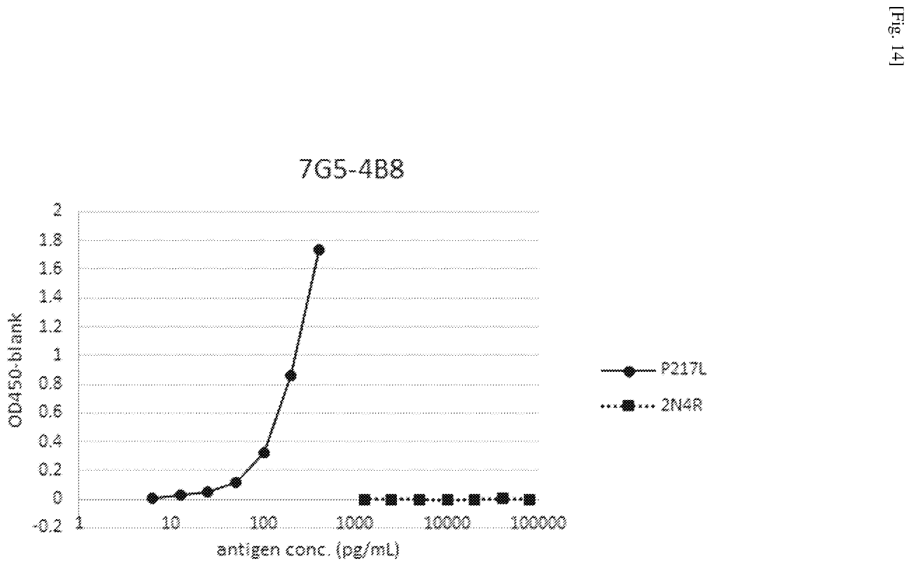

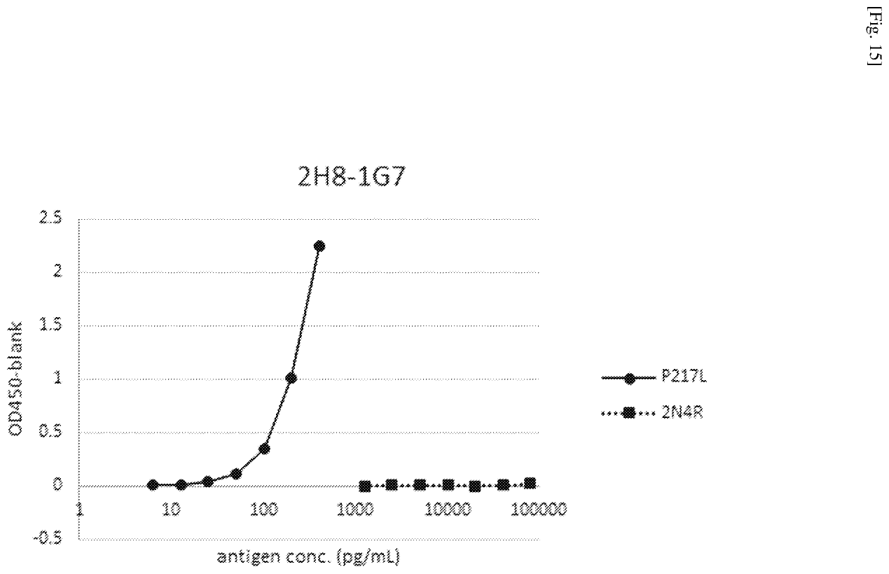

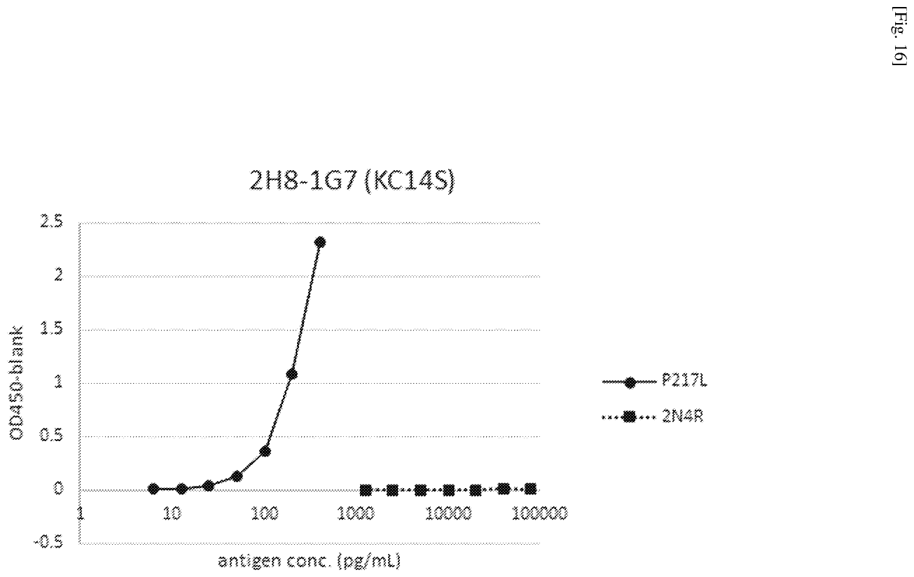

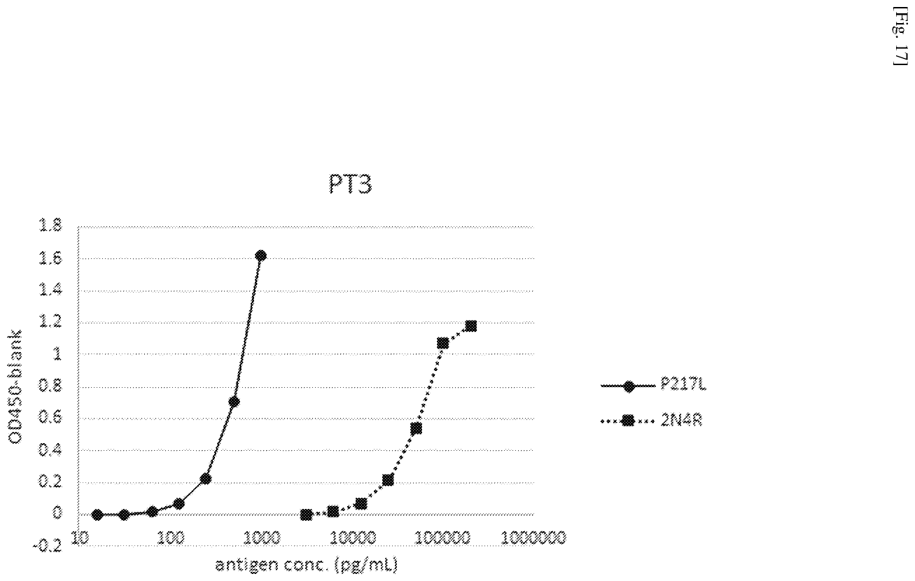

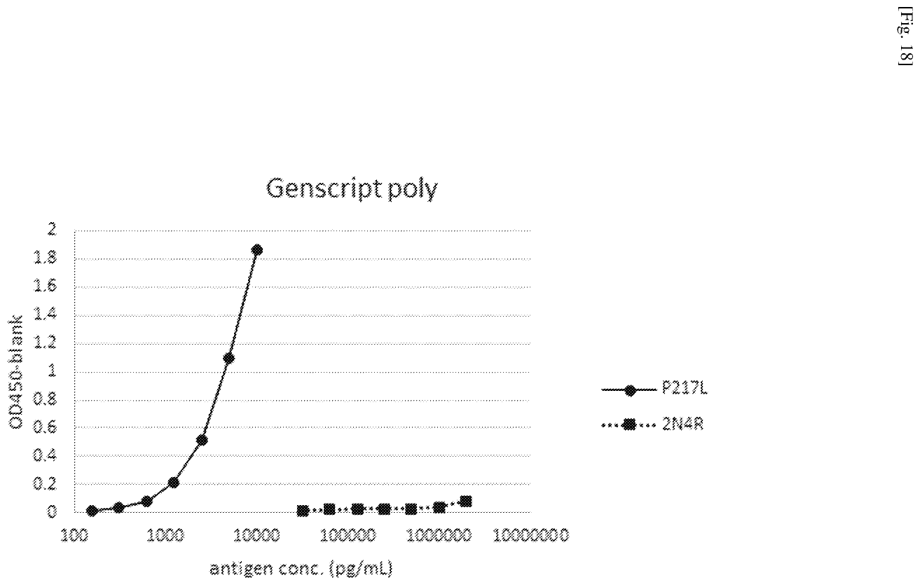

FIG. 13-18 show the calibration curves for 5F6-4D1, 7G5-4B8, 2H8-1G7, 2H8-1G7(KC14S), PT3 and Genscript poly against pT217 Tau peptide (P217L) respectively, and 200-fold concentration (16.3-fold as molecular number) of non-phosphorylated Tau protein (2N4R). The sandwich ELISA with 5F6-4D1, 7G5-4B8, 2H8-1G7 and 2H8-1G7(KC14S) reacted well against pT217 Tau peptide but they didn't react with non-phosphorylated Tau protein at all even in 200-fold higher concentration (16.3-fold as molecular number). The sandwich ELISA with Genscript poly reacted against pT217 Tau peptide by low sensitive manner but they have almost no reaction to non-phosphorylated Tau protein even in 200-fold higher concentration (16.3-fold as molecular number). On the other hand, the sandwich ELISA with PT3 reacted against pT217 Tau peptide and also to non-phosphorylated Tau protein (about 10 to 20-fold stronger to pT217 Tau peptide against non-phosphorylated Tau protein as molecular number). 5F6-4D1, 7G5-4B8, 2H8-1G7 and 2H8-1G7(KC14S) have high selectivity against pT217 Tau sequence.

### Example 9: Selectivity to pT217 Tau Sequence by Inhibition Test in Sandwich ELISA

Anti-Tau monoclonal antibody BT2 (Thermo Fisher Scientific) was adjusted with 50 mM Tris/HCl (pH 7.5) to a concentration of 1 microgram/mL, and then injected at 100 microliter/well in a Maxisorp cup (NUNC). The cup was placed in a humid box and coated overnight at 4 degrees Celsius. After the antibody solution was removed by aspiration, a blocking solution (5% skimmed milk (FUJIFILM Wako Pure Chemical), 50 mM Tris/HCl (pH 7.5), 150 mM NaCl, 0.1% sodium azide) was injected at 200 microliter/well, followed by blocking at room temperature for 2 hours or at 4 degrees Celsius for one day or more (hereinafter referred to as “BT2 cup”). The test recombinant antibodies: 5F6-4D1, 7G5-4B8, 2H8-1G7, 2H8-1G7(KC14S) and comparator antibodies: PT3 and anti-pT217 Tau polyclonal antibody (Genscript poly; Cat. No. A00896, Genscript) were labeled with peroxidase using a Peroxidase Labeling Kit-NH2 (DOJINDO MOLECULAR TECHNOLOGIES) in accordance with the instruction manual attached to the kit (hereinafter referred to as “peroxidase-labeled pT217 Tau antibodies”).

The BT2 cup was washed three times with a washing solution (50 mM Tris/HCI (pH 7.5), 150 mM NaCl, 0.01% Tween®-20), and a long pT217 Tau peptide (KSGDRSGYSSPGSPGTPGSRSRTPSLP(PT)PPTREPKK (SEQ ID NO: 73); P217L) which had been diluted to 120, 150, 250, 250, 1,500 and 15,000 pg/mL each for 5F6-4D1, 7G5-4B8, 2H8-1G7, 2H8-1G7(KC14S), PT3 and Genscript poly with a reaction buffer (5% skimmed milk (FUJIFILM Wako Pure Chemical), 50 mM Tris/HCI (pH 7.5), 150 mM NaCl, 0.2% EDTA-3Na, 0.01% Tween®-20, 4% polyethylene glycol 6000, 0.2% ProClin 150 (SUPELCO, 49376-U, Sigma-Aldrich) was injected at 100 microliter/well and reacted at room temperature for 2 hours. After washing three times with the washing solution, the peroxidase-labeled pT217 Tau antibodies which had been diluted to 100 ng/ml with a reaction buffer which contained 0, 0.24, 0.98, 3.90, 15.6, 62.5, 250 and 1,000 ng/ml of T17P short peptide (RSRTPSLP(PT)PPTREPKK (SEQ ID NO: 74)) or T17 short peptide (RSRTPSLPTPPTREPKK (SEQ ID NO: 75)) was injected at 100 microliter/well and reacted at room temperature for 1 hour. After washing three times with the washing solution, a 3,3′,5,5′-Tetramethylbenzidine (TMB) Liquid Substrate System for ELISA (KPL, SeraCare Life Sciences) was injected at 100 microliter/well and reacted at room temperature for 30 minutes. 0.5 M H₂SO₄ was injected at 100 microliter/well to stop the reaction, and OD (450 to 650 nm) was then measured for cach well.

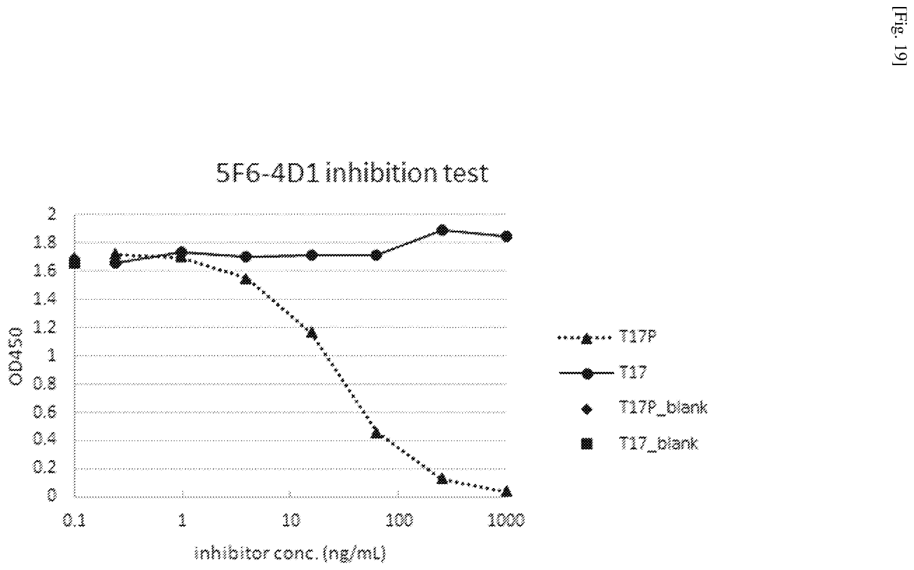

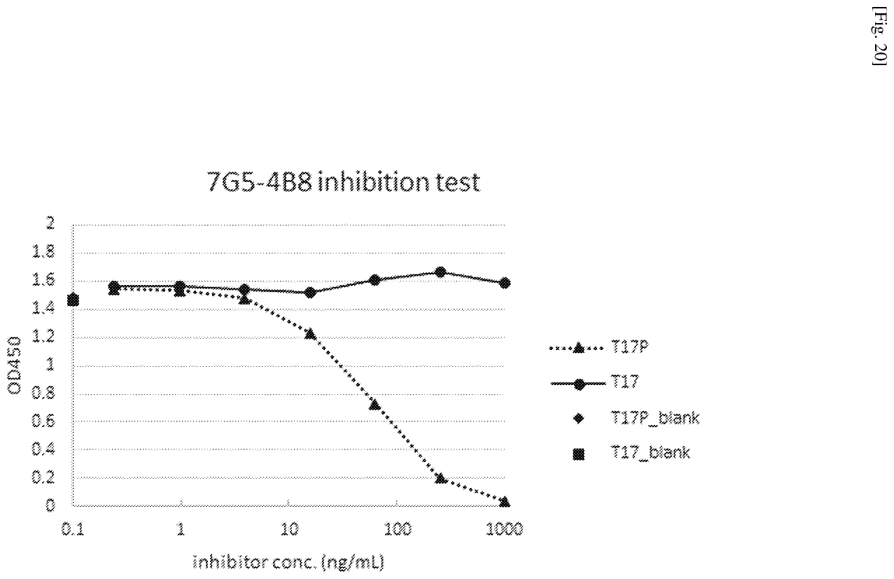

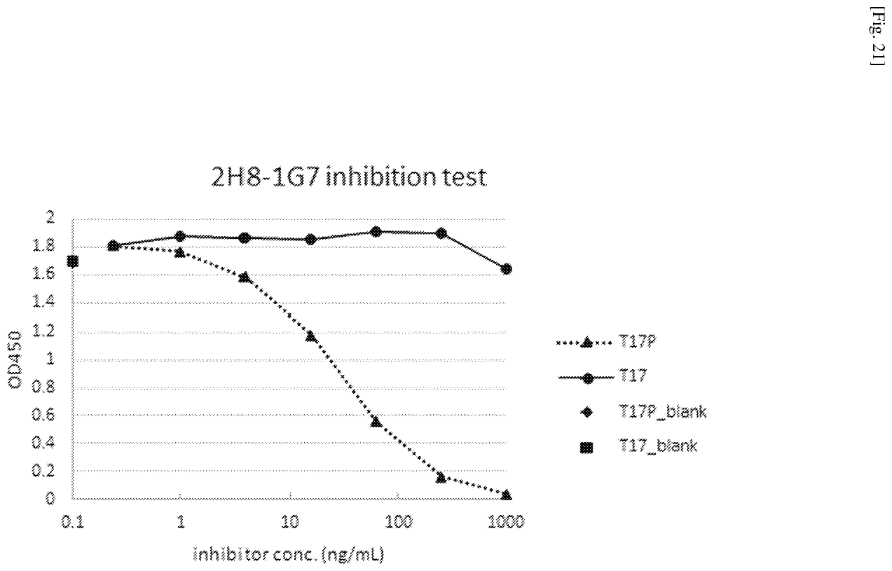

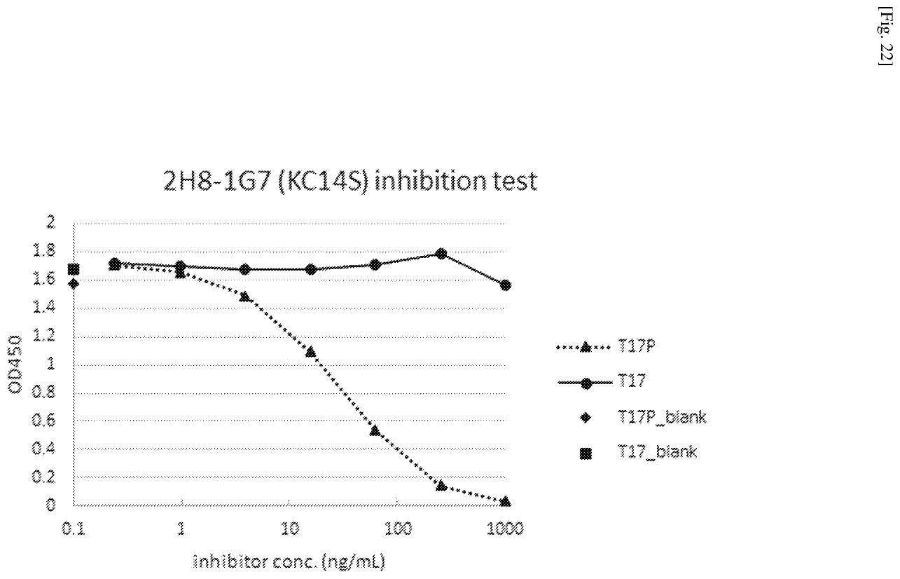

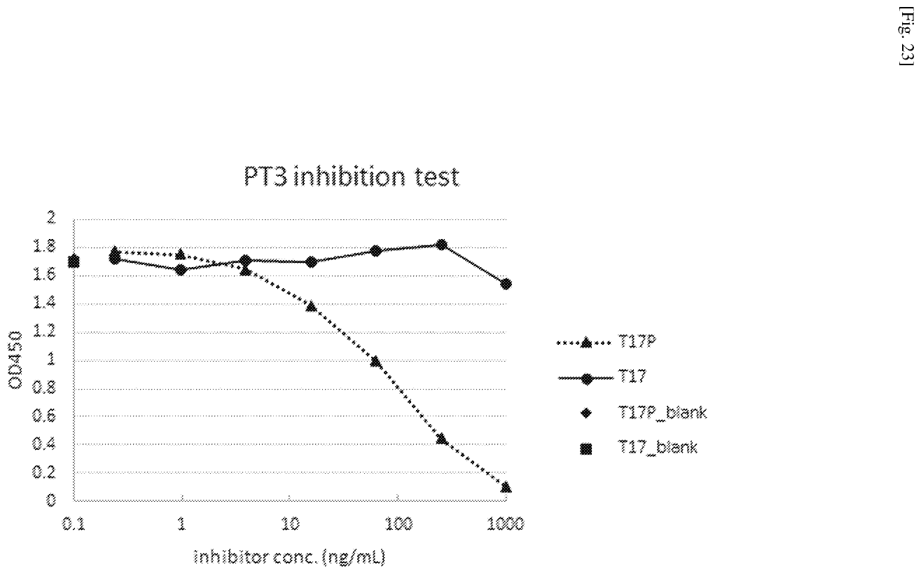

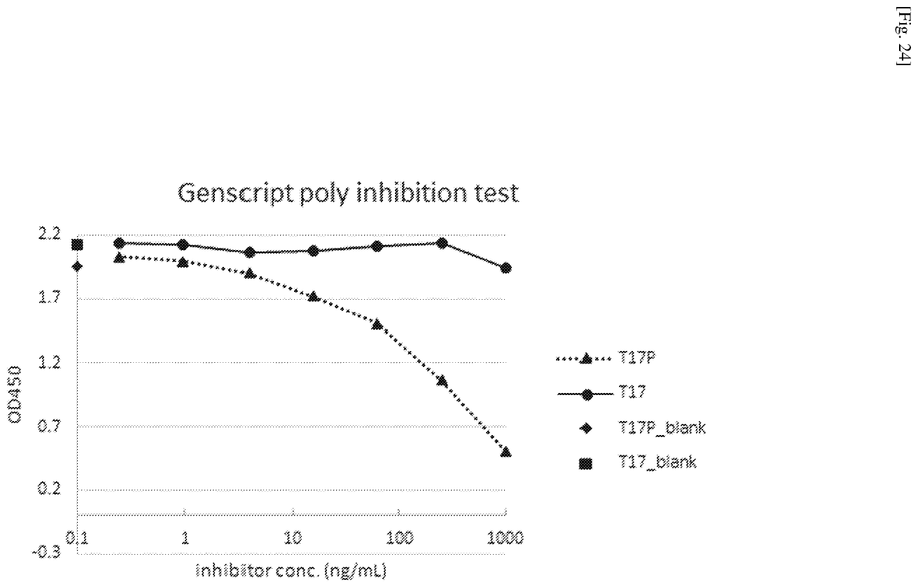

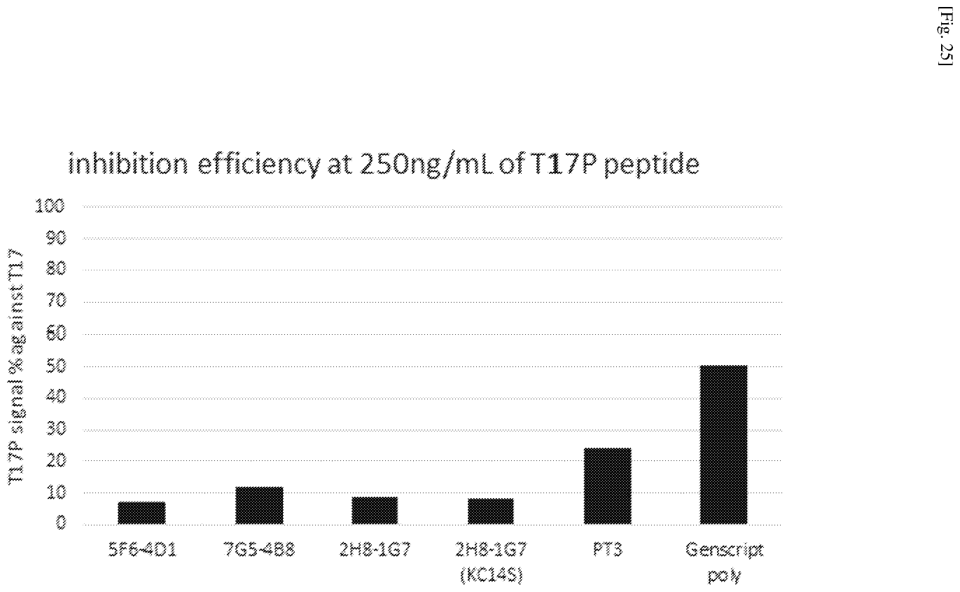

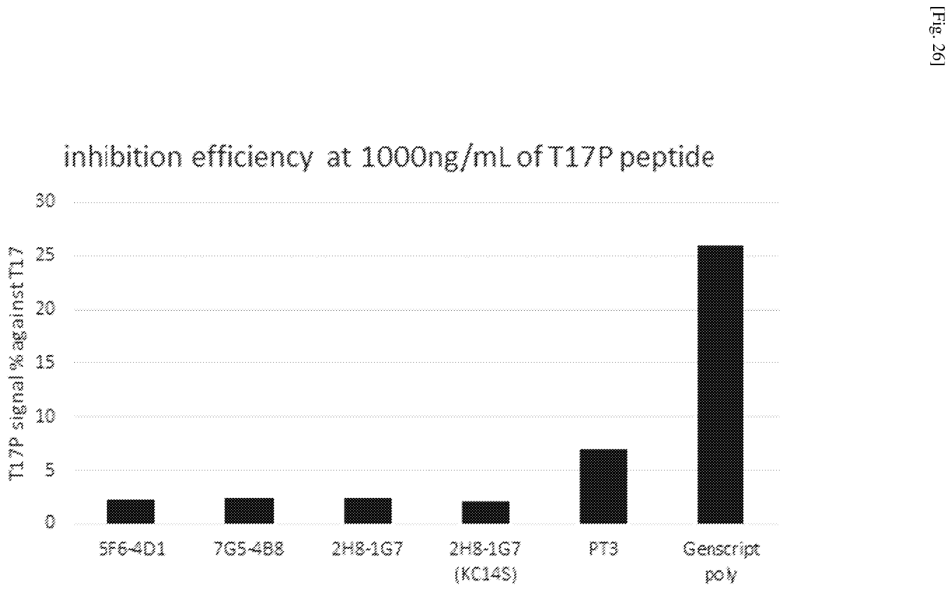

FIGS. 19-26 show the inhibition rate of 5F6-4D1, 7G5-4B8, 2H8-1G7, 2H8-1G7(KC14S), PT3 and Genscript poly by T17P or T17 peptide. The ELISA signals of 5F6-4D1, 7G5-4B8, 2H8-1G7 and 2H8-1G7(KC14S) were inhibited by T17P peptide by dose dependent manner and inhibited more than 97.5% in concentration of 1,000 ng/mL, but were inhibited scarcely by T17 peptide even in concentration of 1,000 ng/mL. On the other hand, the ELISA signal of PT3 and Genscript poly was also inhibited scarcely by T17 peptide but was inhibited rather weakly by T17P peptide about 93% and 74% in concentration of 1,000 ng/ml. 5F6-4D1, 7G5-4B8, 2H8-1G7 and 2H8-1G7(KC14S) were confirmed to have high selectivity against pT217 Tau sequence by the inhibition test.

Example 10: Expression and Purification of Recombinant anti-pT217 Tau Antibodies

Gene encoding heavy chain of antibody 2D6-2A2 (SEQ ID NO: 5) and gene encoding light chain of antibody 2D6-2A2 (SEQ ID NO: 10) were fused by gene encoding N-terminal signal sequence of heavy chain (amino acid: MEWSWVFLFFLSVTTGVHS; SEQ ID NO: 69; nucleotide: ATGGAATGGTCCTGGGTGTTCCTGTTCTTCCTGAGCGTGACAACCGGCGTGCACAGC; SEQ ID NO: 76) and gene encoding N-terminal signal sequence of light chain (amino acid: MSVPTQVLGLL-LLWLTDARC; SEQ ID NO: 71; nucleotide: ATGAGCGTGCCTACACAGG-GCTGGGCCTGCTCCTGCTGTGGCTGACCGACGCTAGATGT; SEQ ID NO: 77), respectively. Gene encoding heavy chain of antibody 7D11-1D10 (SEQ ID NO: 27) and gene encoding light chain of antibody 7D11-1D10 (SEQ ID NO: 31) were fused by gene encoding N-terminal signal sequence of heavy chain (amino acid: MEWSWVFLFFLSVTTGVHS; SEQ ID NO: 69; nucleotide: ATGGAATGGAGCTGGGTCTTTCTGTTCTTCCTGAGCGTGACAACCGGCGTGCACAGC; SEQ ID NO: 78) and gene encoding N-terminal signal sequence of light chain (amino acid: MSVPTQVLGLLLLWLTDARC; SEQ ID NO: 71; nucleotide: ATGAGCGTCCCCACACAGGTGCTGGGCCTGCTGCTGCTCTGGCTGACAGATGCCAGATGT; SEQ ID NO: 79), respectively. Fusion genes were synthesized and cloned into pcDNA3.4 expression vector (Thermo Fisher Scientific). Heavy chain constant regions and light chain constant regions of these antibodies were mouse IgG2b and mouse Ig kappa, respectively. 2D6-2A2 and 7D11-1D10 were expressed by transfecting genes to Expi293F cells (Thermo Fisher Scientific), then the antibodies were purified from supernatants by using MabSelect (Cytiva).

TABLE 5
Nucleotide sequences of 2D6-2A2 and 7D11-1D10

| | | SEQ ID | |
|---|---|---|---|
| Clone Name | | NO | Nucleotide Sequence |
| 2D6-2A2 | Heavy Chain | 37 | GACTACGGCATGCAC |
| | CDR1 | | |
| | Heavy Chain | 80 | TACATGAGCAGCGGAAGCTCCACCAT |
| | CDR2 | | CTACTACGTGGACACAGTGAAGGGC |
| | Heavy Chain | 81 | AGACTGTCTATC |
| | CDR3 | | |
| | Heavy Chain | 82 | GAGGTGCAGCTGGTCGAGTCTGGCGG |
| | Variable Domain | | CGGACTGGTGAAACCTGGGGGCTCCC |
| | | | TGAAACTGAGCTGCGCCGCCTCTGGA |
| | | | TTTACACTGAGCGACTACGGCATGCA |
| | | | CTGGGTGAGACAGGCCCCAGAGAAG |
| | | | GGCCTGGAATGGATCGCCTACATGAG |
| | | | CAGCGGAAGCTCCACCATCTACTACG |
| | | | TGGACACAGTGAAGGGCAGATTCACC |
| | | | ATCAGCCGGGACAACGCCAAGAACA |
| | | | CACTGTTTCTGCAGATGACAAGCCTT |
| | | | AGATCTGAGGACACAGCTATGTATTAC |
| | | | TGCGCTAGAAGACTGTCTATCTGGGG |
| | | | CACCGGAACCACCGTGACAGTGAGC |
| | | | AGC |
| | Heavy Chain | 83 | GAGGTGCAGCTGGTCGAGTCTGGCGG |
| | | | CGGACTGGTGAAACCTGGGGGCTCCC |
| | | | TGAAACTGAGCTGCGCCGCCTCTGGA |
| | | | TTTACACTGAGCGACTACGGCATGCA |
| | | | CTGGGTGAGACAGGCCCCAGAGAAG |
| | | | GGCCTGGAATGGATCGCCTACATGAG |
| | | | CAGCGGAAGCTCCACCATCTACTACG |
| | | | TGGACACAGTGAAGGGCAGATTCACC |
| | | | ATCAGCCGGGACAACGCCAAGAACA |
| | | | CACTGTTTCTGCAGATGACAAGCCTT |
| | | | AGATCTGAGGACACAGCTATGTATTAC |
| | | | TGCGCTAGAAGACTGTCTATCTGGGG |
| | | | CACCGGAACCACCGTGACAGTGAGC |
| | | | AGCGCCAAGACCACCCCTCCCAGCGT |
| | | | CTACCCCCTGGCCCCAGGCTGCGGCG |
| | | | ACACCACAGGAAGCAGCGTGACCCT |
| | | | GGGCTGTCTGGTGAAAGGCTACTTCC |
| | | | CTGAAAGCGTGACCGTGACATGGAAC |
| | | | AGCGGCAGTCTCAGCAGCTCTGTCCA |
| | | | CACCTTCCCCGCTCTGCTGCAATCTGG |
| | | | CCTCTACACCATGTCATCTAGCGTGAC |
| | | | TGTCCCCAGTAGCACCTGGCCTAGCC |
| | | | AGACAGTGACATGCAGCGTGGCCCAC |
| | | | CCCGCCAGCAGCACCACCGTGGACA |
| | | | AGAAGCTGGAACCTAGCGGTCCTATC |
| | | | TCCACAATCAACCCTTGCCCTCCATGT |
| | | | AAAGAGTGCCACAAGTGTCCTGCTCC |
| | | | AAACCTGGAAGGCGGCCCTAGCGTTT |
| | | | TCATCTTCCCTCCTAATATCAAGGACG |
| | | | TGCTGATGATCAGCCTGACACCTAAG |
| | | | GTGACCTGTGTGGTGGTGGATGTGTC |
| | | | TGAGGACGACCCTGATGTGCGCATCT |
| | | | CTTGGTTCGTGAACAACGTGGAAGTG |
| | | | CATACCGCCCAGACCCAGACCCACAG |
| | | | AGAGGATTACAACACCACCATTAGAG |
| | | | TGGTGTCCGCCCTGCCCATCCAACAC |
| | | | CAGGACTGGATGAGCGGAAAGGAGT |
| | | | TCAAGTGCAAGGTTAACAACAAGGAC |
| | | | CTGCCTAGCCCCATCGAGCGGACCAT |
| | | | CTCTAAAATCAAGGGCCTGGTGCGGG |
| | | | CCCCCCAGGTGTACATCCTGCCTCCTC |
| | | | CCGCCGAGCAGCTGAGCAGGAAGGA |
| | | | TGTGAGCCTGACCTGCCTGGTGGTGG |
| | | | GCTTCAACCCTGGCGATATCAGCGTC |
| | | | GAGTGGACCTCTAATGGCCACACGGA |
| | | | AGAGAACTATAAGGACACCGCCCCTG |
| | | | TGCTGGACTCCGACGGCAGCTACTTT |
| | | | ATCTACAGCAAGCTGGATATCAAGAC |
| | | | CAGCAAGTGGGAGAAGACAGATAGC |
| | | | TTCAGCTGCAACGTGCGGCACGAGGG |
| | | | CCTGAAGAACTACTACCTGAAGAAAA |
| | | | CCATTAGCAGAAGCCCAGGCAAG |
| | Light Chain | 84 | AGCGCCAGCAGCAGCGTGTCTAGCAT |
| | CDR1 | | GTAC |
| | Light Chain | 85 | GACACCTCTAATCTGGCCTCT |
| | CDR2 | | |
| | Light Chain | 86 | CAGCAGTGGTCCAGCTACCCTCTGAC |
| | CDR3 | | C |
| | Light Chain | 87 | CAAATCGTGCTGACACAGAGCCCTGC |
| | Variable Domain | | CATCATGTCCGCCAGCCCCGGCGAGA |
| | | | AGGTGACAATGACCTGTAGCGCCAGC |
| | | | AGCAGCGTGTCTAGCATGTACTGGTA |
| | | | CCACCAGAAGCCTGGCTCTTCTCCAA |
| | | | GACTGCTGATCTACGAGACCTCTAATC |
| | | | TGGCCTCTGGAGTCCCCGTGCGGTTC |
| | | | AGCGGCTCTGGCAGCGGCACATCATA |
| | | | TAGCCTGACAATCAGCAGAATGGAAG |
| | | | CTGAAGATGCCGCTACATACTACTGCC |
| | | | AGCAGTGGTCCAGCTACCCTCTGACC |
| | | | TTTGGAGCCGGCACCAAGCTGGAACT |
| | | | GAAG |
| | Light Chain | | CAAATCGTGCTGACACAGAGCCCTGC |
| | | | CATCATGTCCGCCAGCCCCGGCGAGA |
| | | | AGGTGACAATGACCTGTAGCGCCAGC |
| | | | AGCAGCGTGTCTAGCATGTACTGGTA |
| | | | CCACCAGAAGCCTGGCTCTTCTCCAA |
| | | | GACTGCTGATCTACGACACCTCTAATC |
| | | | TGGCCTCTGGAGTCCCCGTGCGGTTC |
| | | | AGCGGCTCTGGCAGCGGCACATCATA |
| | | | TAGCCTGACAATCAGCAGAATGGAAG |
| | | | CTGAAGATGCCGCTACATACTACTGCC |
| | | | AGCAGTGGTCCAGCTACCCTCTGACC |
| | | | TTTGGAGCCGGCACCAAGCTGGAACT |
| | | | GAAGCGGGCCGACGCCGCCCCTACCG |
| | | | TGTCCATCTTCCCACCTAGCTCTGAAC |
| | | | AGCTGACCAGCGGAGGCGCCTCTGTT |
| | | | GTGTGCTTCCTGAACAACTTCTACCCC |
| | | | AAGGACATCAACGTGAAGTGGAAGAT |
| | | | CGACGGCAGCGAGCGGCAGAACGGC |
| | | | GTGCTGAATAGCTGGACAGATCAGGA |
| | | | CTCCAAGGATAGCACCTACAGCATGA |
| | | | GCAGCACCCTGACCCTGACCAAAGAC |
| | | | GAGTACGAGAGACACAACACCTACA |
| | | | CCTGCGAGGCCACACACAAGACCTCC |
| | | | ACCAGCCCTATTGTGAAGAGCTTCAA |
| | | | CAGAAACGAGTGC |
| 7D11-1D10 | Heavy Chain | 37 | GACTACGGCATGCAC |
| | CDR1 | | |
| | Heavy Chain | 89 | TACATCAGCTCTGGCGACTOCACAATT |
| | CDR2 | | TACTACGCCGACACCGTGAAGGGA |
| | Heavy Chain | 90 | AGAATGGCCTAC |
| | CDR3 | | |
| | Heavy Chain | 91 | GAGGTGCAACTGGTTGAGAGCGGCG |
| | Variable Domain | | GTGGCCTGGTGAAACCTGGCGGCTCT |
| | | | CTGAAGCTGAGCTGCGCCCCCTCTGG |
| | | | CTTCACACTGTCTGACTACGGCATGC |
| | | | ACTGGGTGAGACAGGCTCCAGAGAA |
| | | | AGGACTCGAGTGGTTCGCCTACATCA |
| | | | GCTCTGGCGACTCCACAATTTACTACG |
| | | | CCGACACCGTGAAGGGAAGATTCACC |
| | | | ATCTCTAGGGATAACGCCAAGAACAC |
| | | | CCTGTTTCTGCAGATGACCAGCCTGA |
| | | | GATCTGAGGACACAGCTATGTACTACT |
| | | | GCGCCAGAAGAATGGCCTACTGGGGG |
| | | | CAGGGCACCCTGGTCACCGTGTCTGC |
| | | | C |
| | Heavy Chain | 92 | GAGGTGCAACTGGTTGAGAGCGGCG |
| | | | GTGGCCTGGTGAAACCTGGCGGCTCT |
| | | | CTGAAGCTGAGCTGCGCCGCCTCTGG |
| | | | CTTCACACTGTCTGACTACGGCATGC |
| | | | ACTGGGTGAGACAGGCTCCAGAGAA |
| | | | AGGAGTCGAGTGGTTCGCCTACATCA |
| | | | GCTCTGGCGACTCCACAATTTACTACG |
| | | | CCGACACCGTGAAGGGAAGATTCACC |
| | | | ATCTCTAGGGATAACGCCAAGAACAC |
| | | | CCTGTTTCTGCAGATGACCAGCCTGA |
| | | | GATCTGAGGACACAGCTATGTACTACT |
| | | | GCGCCAGAAGAATGGCCTACTGGGGC |
| | | | CAGGGCACCCTGGTCACCGTGTCTGC |
| | | | CGCTAAGACCACACCCCCTAGCGTTT |
| | | | ATCCTCTGGCCCCTGGATGTGGCGATA |
| | | | CCACCGGATCAAGCGTGACACTGGGC |
| | | | TGCCTAGTGAAGGGCTACTTTCCTGA |
| | | | GTCTGTGACCGTGACATGGAACAGCG |
| | | | GCAGCCTGTCTTCTTCTGTACATACAT |
| | | | TCCCAGCTCTTCTGCAGAGCGGCCTG |
| | | | TACACCATGAGCTCCAGCGTGACAGT |
| | | | GCCCAGCTCCACATGGCCTAGCCAGA |
| | | | CCGTGACGTGTAGCGTGGCCCACCCC |
| | | | GCCAGCAGCACCACCGTGGACAAGA |
| | | | AGCTGGAACCTAGCGGACCTATCAGC |
| | | | ACCATTAACCCTTGCCCCCCCTGCAA |
| | | | GGAATGCCACAAGTGCCCTGCCCCTA |
| | | | ATCTGGAAGGCGGCCCTAGCGTGTTC |
| | | | ATCTTCCCCCCCAATATCAAGGACGTG |
| | | | CTGATGATCTCTCTGACCCCTAAGGTT |
| | | | ACATGCGTGGTGGTGGATGTGTCCGA |
| | | | GGACGACCCCGACGTGCGGATCTCCT |
| | | | GGTTCGTGAACAACGTGGAAGTGCAC |
| | | | ACCGCGCAGACACAGACCCACCGGG |
| | | | AAGATTACAATTCTACAATCCGGGTGG |
| | | | TCTCCGCCCTGCCAATCCAGCACCAG |
| | | | GACTGGATGAGCGGAAAGGAGTTCA |
| | | | AGTGTAAAGTTAACAACAAGGACCTG |
| | | | CCTAGTCCTATCGAGAGAACCATCAG |
| | | | CAAGATCAAGGGCCTGGTGCGGGCCC |
| | | | CTCAAGTGTACATCCTGCCACCTCCTG |
| | | | CCGAGCAGCTGAGCAGAAAGGATGT |
| | | | GAGCCTGACCTGTCTGGTGGTCGGAT |
| | | | TTAACCCCGGCGACATCTCCGTGGAA |
| | | | TGGACCAGCAACGGCCACACCGAAG |
| | | | AGAACTACAAGGACACCGCCCCTGTG |
| | | | CTGGACAGCGACGGCAGCTACTTCAT |
| | | | CTACTCCAAACTCGATATCAAAACCA |
| | | | GCAAGTGGGAGAAGACTGATAGCTTC |
| | | | AGCTGCAACGTGCGCCACGAGGGCCT |
| | | | GAAAAATTACTATCTGAAGAAGACCA |
| | | | TCAGCAGAAGCCCTGGCAAG |
| | Light Chain | 93 | AGCGCCTCTAGCAGCGTGACCTACAT |
| | CDR1 | | GTAC |
| | Light Chain | 94 | GCCACCAGCAACCTGGCCTCT |
| | CDR2 | | |
| | Light Chain | 95 | CACCAGTGGTCCTCCTACCCCCTGAC |
| | CDR3 | | C |
| | Light Chain | 96 | CAGATCGTGCTGACCCAGAGCCCTGC |
| | Variable Domain | | CATCATGTCTGCCAGCCCTGGCGAGA |
| | | | AGGTGACAATGACCTGTAGCGCCTCT |
| | | | AGCAGCGTGACCTACATGTACTGGTA |
| | | | CCAGCAAAAGCCTGGAAGCTCTCCCA |
| | | | GACTGCTGATCTACGCCACCAGCAAC |
| | | | CTGGCCTCTGGAGTGCCTGTGCGGTT |
| | | | CAGCGGCTCTGGCAGCGGAACTAGCT |
| | | | ACAGCCTGACCATCAGCAGAATGGAA |
| | | | GCTGAAGATGCCGCCACCTACTACTG |
| | | | CCAGCAGTGGTCCTCCTACCCCCTGA |
| | | | CCTTCGGCGCCGGCACAAAGCTGGAA |
| | | | CTGAAG |
| | Light Chain | 97 | CAGATCGTGCTGACCCAGAGCCCTGC |
| | | | CATCATGTCTGCCAGCCCTGGCGAGA |
| | | | AGGTGACAATGACCTGTAGCGCCTCT |
| | | | AGCAGCGTGACCTACATGTACTGGTA |
| | | | CCAGCAAAAGCCTGGAAGCTCTCCCA |
| | | | GACTGCTGATCTACGCCACCAGCAAC |
| | | | CTGGCCTCTGGAGTGCCTGTGCGGTT |
| | | | CAGCGGCTCTGGCAGCGGAACTAGCT |
| | | | ACAGCCTGACCATCAGCAGAATGGAA |
| | | | GCTGAAGATGCCGCCACCTACTACTG |
| | | | CCAGCAGTGGTCCTCCTACCCCCTGA |
| | | | CCTTCGGCGCCGGCACAAAGCTGGAA |
| | | | CTGAAGCGGGCCGACGCCGCTCCTAC |
| | | | AGTGTCCATCTTTCCACCTAGCAGTGA |
| | | | ACAGCTGACATCTGGCGGCGCCAGCG |
| | | | TGGTGTGCTTCCTGAACAACTTCTACC |
| | | | CTAAGGACATTAACGTGAAATGGAAG |
| | | | ATCGACGGCAGCGAGCGGCAGAACG |
| | | | GCGTGCTGAATAGCTGGACCGACCAA |
| | | | GACAGCAAGGATAGCACCTATTCTATG |
| | | | TCCAGCACCCTGACACTGACCAAGGA |
| | | | CGAGTACGAGAGACACAACAGCTATA |
| | | | CATGCGAGGCTACCCACAAGACCTCC |
| | | | ACCAGCCCCATCGTTAAGAGCTTCAA |
| | | | TAGAAACGAGTGC |

### Example 11: Kinetic Analysis of anti-pT217 Tau Monoclonal Antibodies Binding to Synthesized Biotinylated Tau Phosphopeptides

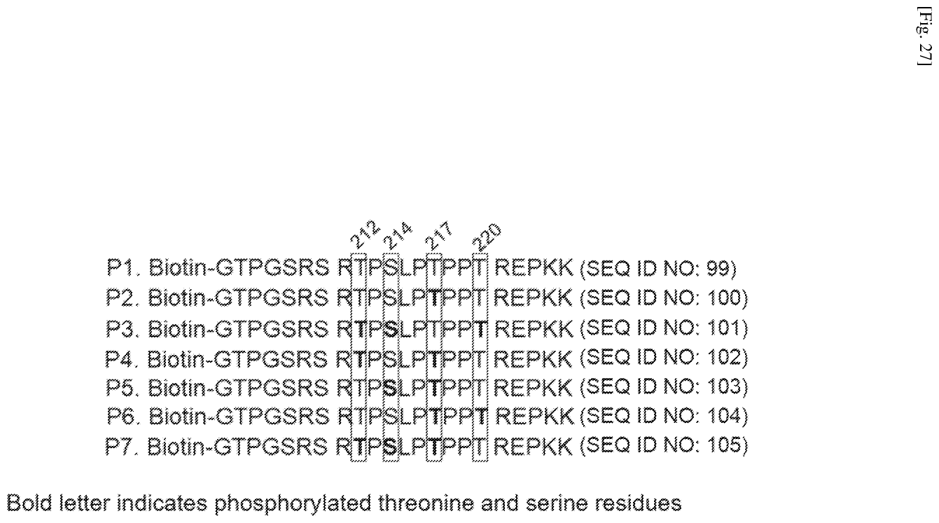

Kinetics of anti-pT217 Tau recombinant monoclonal antibodies interaction with biotinylated tau peptide was investigated using BIAcore S200 (Cytiva). The non-phosphorylated and phosphorylated tau peptides contained biotin moiety at the N-terminus were synthesized (Scrum). The panel of peptides covered tau residues 204-225 (GTPGSRSRTPSLPTPPTREPKK; SEQ ID NO: 98) and included combinations of phosphorylation at the following sites: T212, S214, T217, and T220 (FIG. 27).

The interaction was analyzed as shown below. Biotinylated peptides were captured on a carboxymethylated dextran matrix pre-immobilized with NeutraAvidin (Cytiva). Purified anti-pT217 Tau monoclonal antibodies: 5F6-4B4, 7G5-4B8, 2H8-1G7, 2H8-1G7(KC14S), 2D6-2A2, 7D11-1D10, and comparator antibodies: PT3 were flowed over the sensor chip at five different concentrations and their relative association rates (Kₐ), dissociation rates (Kₐ), and equilibrium dissociation constants (K_{D}) were calculated in accordance with the manufacturer's instructions. The results are shown in the Tables 6-12.

TABLE 6
Calculated affinity and kinetic parameters to biotinylated
non-phosphorylated tau peptide (P1)

| | Phosphor- | | | |
|---|---|---|---|---|
| | ylation | Ka | Kd | KD |
| Antibody | site | (1/Ms) | (1/s) | (M) |
| 5F6-4D1 | — | nd | nd | nd |
| 7G5-4B8 | — | nd | nd | nd |
| 2H8-1G7 | — | nd | nd | nd |
| 2H8-1G7 | — | nd | nd | nd |
| (KC14S) | | | | |
| 2D6-2A2 | — | nd | nd | nd |
| 7D11-1D10 | — | nd | nd | nd |
| PT3 | — | 1.28 × 10⁷ | 1.66 × 10⁻² | 1.29 × 10⁻⁹ |

nd; not determined (the experiments were conducted; however, no kinetic data were obtained)

TABLE 7
Calculated affinity and kinetic parameters
to biotinylated tau phosphopeptide (P2)

| | Phosphor- | | | |
|---|---|---|---|---|
| | ylation | Ka | Kd | KD |
| Antibody | site | (1/Ms) | (1/s) | (M) |
| 5F6-4D1 | pT217 | 1.71 × 10⁶ | 4.84 × 10⁻⁵ | 2.84 × 10⁻¹¹ |
| 7G5-4B8 | pT217 | 2.15 × 10⁶ | 1.85 × 10⁻⁴ | 8.61 × 10⁻¹¹ |
| 2H8-1G7 | pT217 | 1.35 × 10⁶ | 3.88 × 10⁻⁵ | 2.88 × 10⁻¹¹ |
| 2H8-1G7 | pT217 | 1.43 × 10⁶ | 5.70 × 10⁻⁵ | 3.98 × 10⁻¹¹ |
| (KC14S) | | | | |
| 2D6-2A2 | pT217 | 1.82 × 10⁶ | 1.39 × 10⁻⁴ | 7.62 × 10⁻¹¹ |
| 7D11- | pT217 | 2.56 × 10⁶ | 1.71 × 10⁻⁴ | 6.66 × 10⁻¹¹ |
| 1D10 | | | | |
| PT3 | pT217 | 1.75 × 10⁶ | 3.43 × 10⁻⁴ | 1.96 × 10⁻¹⁰ |

TABLE 8
Calculated affinity and kinetic parameters
to biotinylated tau phosphopeptide (P3)

| | Phosphor- | | | |
|---|---|---|---|---|
| | ylation | Ka1 | Kd1 | KD1 |
| Antibody | site | (1/Ms) | (1/s) | (M) |
| 5F6-4D1 | pT212pS214pT220 | 7.46 × 10⁵ | 5.16 × 10⁻¹ | 6.93 × 10⁻⁷ |
| 7G5-4B8 | pT212pS214pT220 | 3.81 × 10⁴ | 1.88 × 10⁻² | 4.95 × 10⁻⁷ |
| 2H8-1G7 | pT212pS214pT220 | 5.28 × 10⁴ | 4.61 × 10⁻³ | 8.74 × 10⁻⁸ |
| 2H8-1G7 | pT212pS214pT220 | 1.45 × 10⁵ | 8.58 × 10⁻³ | 5.92 × 10⁻⁸ |
| (KC14S) | | | | |
| 2D6-2A2 | pT212pS214pT220 | 7.45 × 10⁶ | 8.76 × 10⁻² | 1.18 × 10⁻⁸ |
| 7D11- | pT212pS214pT220 | 5.32 × 10⁶ | 1.08 × 10⁻¹ | 2.03 × 10⁻⁸ |
| 1D10 | | | | |
| PT3 | pT212pS214pT220 | 1.07 × 10⁵ | 4.58 × 10⁻² | 4.28 × 10⁻⁷ |

TABLE 9
Calculated affinity and kinetic parameters
to biotinylated tau phosphopeptide (P4)

| | Phosphor- | | | |
|---|---|---|---|---|
| | ylation | Ka | Kd | KD |
| Antibody | site | (1/Ms) | (1/s) | (M) |
| 5F6-4D1 | pT212pT217 | 1.99 × 10⁶ | 3.36 × 10⁻⁴ | 1.69 × 10⁻¹⁰ |
| 7G5-4B8 | pT212pT217 | 2.45 × 10⁶ | 3.23 × 10⁻⁴ | 1.32 × 10⁻¹⁰ |
| 2H8-1G7 | pT212pT217 | 1.55 × 10⁶ | 2.31 × 10⁻⁴ | 1.49 × 10⁻¹⁰ |
| 2H8-1G7 | pT212pT217 | 1.65 × 10⁶ | 1.85 × 10⁻⁴ | 1.12 × 10⁻¹⁰ |
| (KC14S) | | | | |
| 2D6-2A2 | pT212pT217 | 2.11 × 10⁶ | 2.16 × 10⁻⁴ | 1.02 × 10⁻¹⁰ |
| 7D11- | pT212pT217 | 1.82 × 10⁶ | 2.15 × 10⁻⁴ | 1.18 × 10⁻¹⁰ |
| 1D10 | | | | |
| PT3 | pT212pT217 | 1.56 × 10⁶ | 1.65 × 10⁻⁵ | 1.05 × 10⁻¹¹ |

TABLE 10
Calculated affinity and kinetic parameters
to biotinylated tau phosphopeptide (PS)

| | Phosphor- | | | |
|---|---|---|---|---|
| | ylation | Ka | Kd | KD |
| Antibody | site | (1/Ms) | (1/s) | (M) |
| 5F6-4D1 | pS214pT217 | 1.61 × 10⁶ | 4.58 × 10⁻⁵ | 2.84 × 10⁻¹¹ |
| 7G5-4B8 | pS214pT217 | 1.78 × 10⁶ | 9.81 × 10⁻⁵ | 5.50 × 10⁻¹¹ |
| 2H8-1G7 | pS214pT217 | 1.17 × 10⁶ | 2.14 × 10⁻⁵ | 1.84 × 10⁻¹¹ |
| 2H8-1G7 | pS214pT217 | 1.14 × 10⁶ | 7.70 × 10⁻⁶ | 6.74 × 10⁻¹² |
| (KC14S) | | | | |
| 2D6-2A2 | pS214pT217 | 1.55 × 10⁶ | 4.52 × 10⁻⁵ | 2.91 × 10⁻¹¹ |
| 7D11- | pS214pT217 | 2.02 × 10⁶ | 9.29 × 10⁻⁵ | 4.61 × 10⁻¹¹ |
| 1D10 | | | | |
| PT3 | pS214pT217 | 1.02 × 10⁶ | 7.45 × 10⁻⁵ | 7.33 × 10⁻¹¹ |

TABLE 11
Calculated affinity and kinetic parameters
to biotinylated tau phosphopeptide (P6)

| | Phosphor- | | | |
|---|---|---|---|---|
| | ylation | Ka | Kd | KD |
| Antibody | site | (1/Ms) | (1/s) | (M) |
| 5F6-4D1 | pT217pT220 | 2.16 × 10⁶ | 3.03 × 10⁻⁴ | 1.40 × 10⁻¹⁰ |
| 7G5-4B8 | pT217pT220 | 2.71 × 10⁶ | 4.27 × 10⁻⁴ | 1.58 × 10⁻¹⁰ |
| 2H8-1G7 | pT217pT220 | 1.74 × 10⁶ | 3.07 × 10⁻⁴ | 1.76 × 10⁻¹⁰ |
| 2H8-1G7 | pT217pT220 | 1.78 × 10⁶ | 3.12 × 10⁻⁴ | 1.75 × 10⁻¹⁰ |
| (KC14S) | | | | |
| 2D6-2A2 | pT217pT220 | 2.05 × 10⁶ | 3.42 × 10⁻⁴ | 1.67 × 10⁻¹⁰ |
| 7D11- | pT217pT220 | 2.87 × 10⁶ | 3.60 × 10⁻⁴ | 1.26 × 10⁻¹⁰ |
| 1D10 | | | | |
| PT3 | pT217pT220 | nd | nd | nd |

nd; not determined (the experiments were conducted; however, no kinetic data were obtained)

TABLE 12
Calculated affinity and kinetic parameters
to biotinylated tau phosphopeptide (P7)

| | Phosphor- | | | |
|---|---|---|---|---|
| | ylation | Ka | Kd | KD |
| Antibody | site | (1/Ms) | (1/s) | (M) |
| 5F6-4D1 | pT212pS214pT217 | 1.74 × 10⁶ | 1.82 × 10⁻⁴ | 1.05 × 10⁻¹⁰ |
| 7G5-4B8 | pT212pS214pT217 | 1.90 × 10⁶ | 1.93 × 10⁻⁴ | 1.02 × 10⁻¹⁰ |
| 2H8-1G7 | pT212pS214pT217 | 1.19 × 10⁶ | 1.57 × 10⁻⁴ | 1.32 × 10⁻¹⁰ |
| 2H8-1G7 | pT212pS214pT217 | 1.22 × 10⁶ | 7.48 × 10⁻⁵ | 6.15 × 10⁻¹¹ |
| (KC14S) | | | | |
| 2D6-2A2 | pT212pS214pT217 | 1.25 × 10⁶ | 1.57 × 10⁻⁴ | 1.26 × 10⁻¹⁰ |
| 7D11- | pT212pS214pT217 | 1.74 × 10⁶ | 1.98 × 10⁻⁴ | 1.14 × 10⁻¹⁰ |
| 1D10 | | | | |
| PT3 | pT212pS214pT217 | 1.99 × 10⁶ | 8.17 × 10⁻⁶ | 4.11 × 10⁻¹² |

### Example 12: pT217 Tau SIMOA® Assay for CSF Samples

The test pT217 Tau recombinant antibody: 5F6-4D1 coated beads and biotinylated Tau 12 (BioLegend) were prepared using Simoa® Homebrew Assay Development Kit (101354, Quanterix) in accordance with the manufacturer's instructions. Beads were diluted with Beads Diluent Buffer in Simoa® Homebrew Assay Development Kit (101354, Quanterix) and beads numbers were adjusted to about 2,500 for 5F6-4D1 beads and about 5,000 for helper beads (103208, Quanterix). Biotinylated Tau12 (Detector) was diluted with SIMOA® Tau Sample Diluent (103847, Quanterix) to concentration of 3 ug/mL. CSF samples of AD patients, mild cognitive impairment due to Alzheimer's disease (MCI) patients, and control (PrecisionMed) were 20-fold diluted with SIMOA® Tau Sample Diluent (103847, Quanterix).

The measurement of CSF samples was carried out automatically in SIMOA® HD-1 analyzer (Quanterix, Billerica) with assay definition of 2-step assay neat 2.0. The assay condition was described as the below Table 13. SBG (streptavidin-beta-galactosidase) concentrate was diluted with SBG Diluent to 150 pM. SBG Diluent and RGP (resorufin-beta-D-galactopyranoside) were contained in SIMOA® Enzyme and Substrate Kit (101361, Quanterix). The sample AEB (SIMOA® signal count) was transferred to the concentration compare to the AEB of calibrator sample (Tau-441 DYRKIA phosphorylated, T08-50RN, SignalChem Biotech).

TABLE 13
Assay condition of SIMOA assay for CSF samples

| | | Volume | Incubation Time |
|---|---|---|---|
| Step | Liquid | (μL) | (cad*) |
| Step 1 | Beads | 25 | — |
| | Sample | 100 | — |
| | Detector | 20 | 47 |
| Step 2 | SBG | 100 | 7 |
| Measuring | RGP | 25 | — |

*cad = cadences,
1 cad = 45 sec

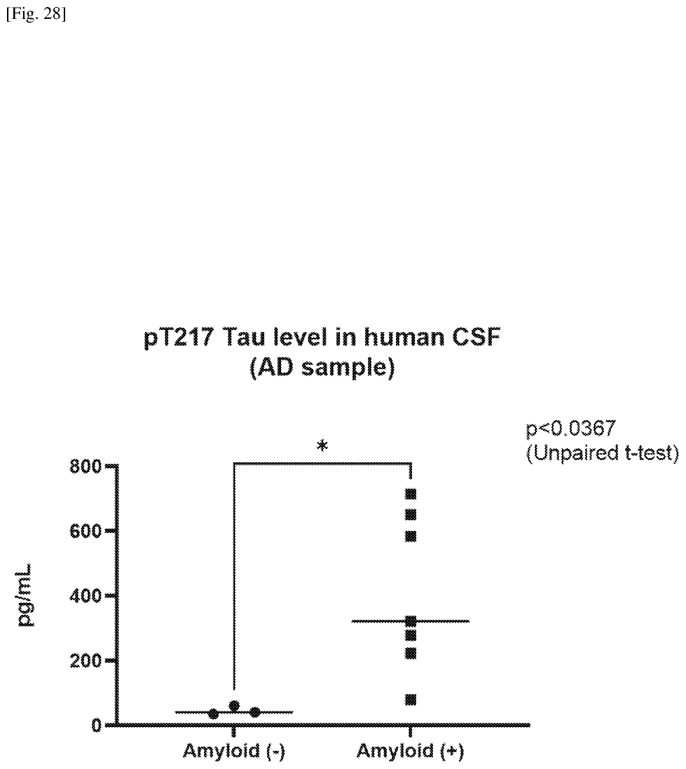

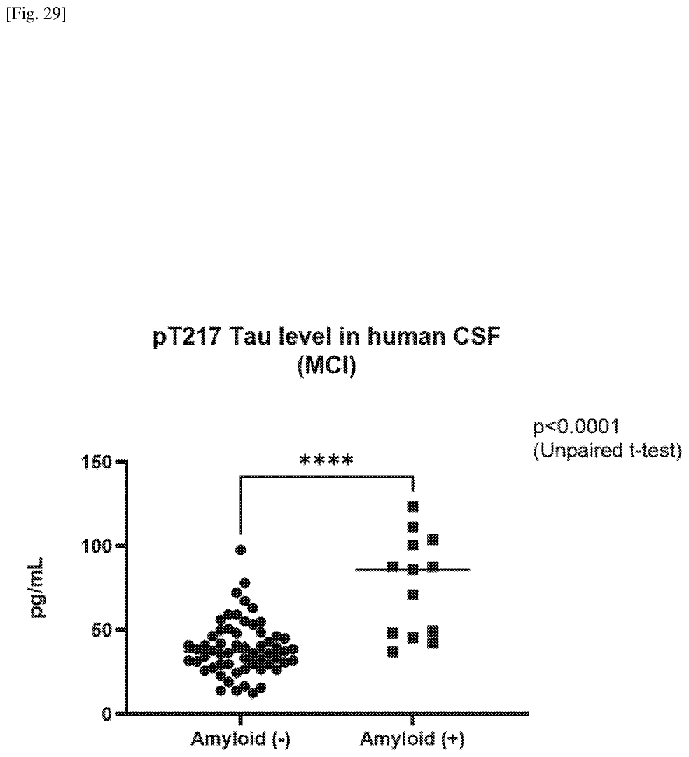

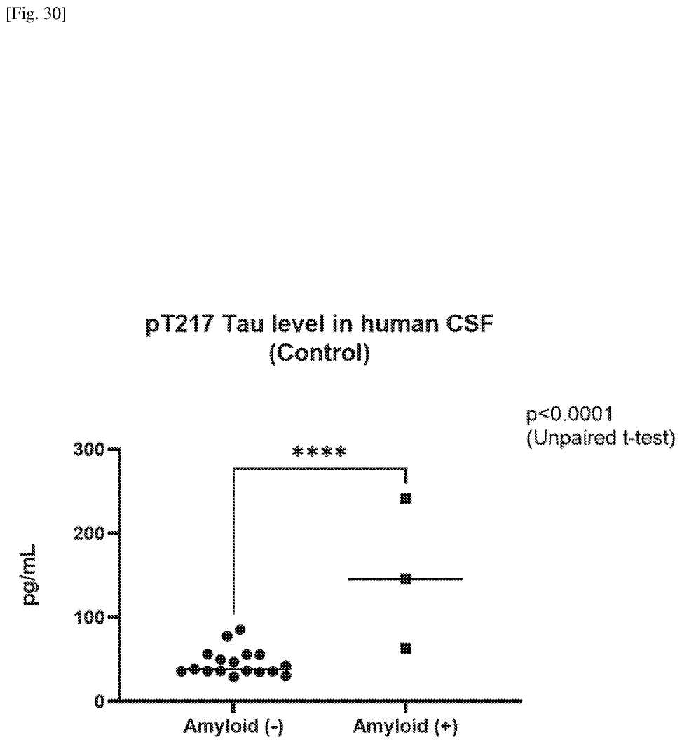

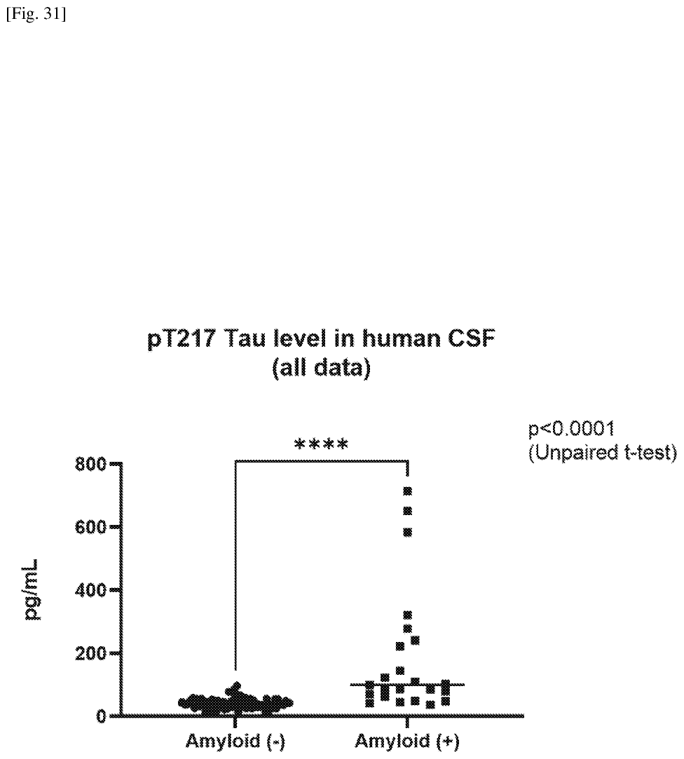

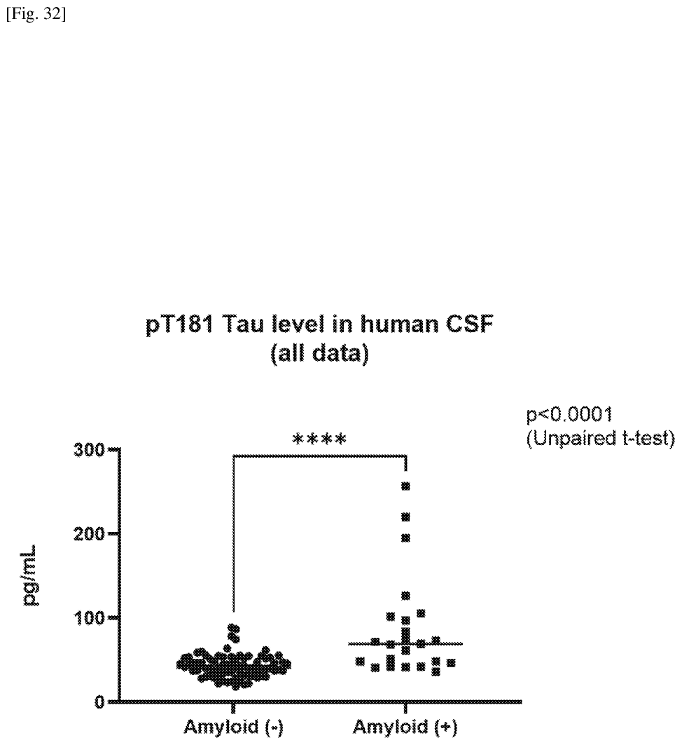

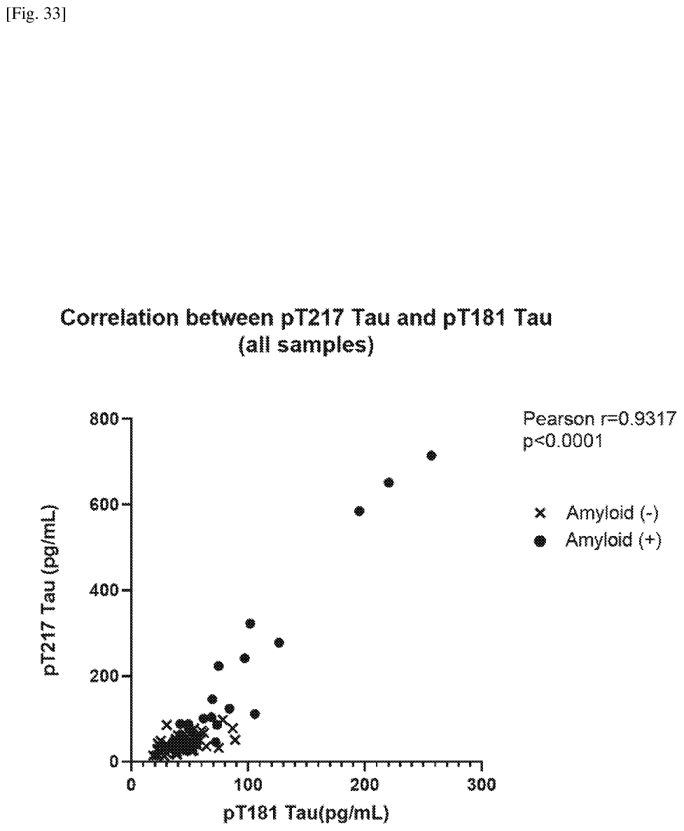

The distributions of CSF pT217 Tau in AD samples, MCI samples, control samples, and all samples were shown in FIG. 28-31. Brain amyloid deposition status of each sample was estimated as positive by CSF total tau (Lumipulse)/Ab42 (Lumipulse) >0.54. The results of CSF pT217 Tau for all samples were compared with the results of CSF pT181 Tau for all samples (FIG. 32) measured with Finoscholar® pT181 Tau (NS-PTU, NIPRO, Japanese distributer of Innotest®) in accordance with kit instruction manual. Statistical analysis was carried out with GraphPad Prism 9.0.2. Significance test was done by unpaired t-test between Amyloid (+) and Amyloid (−) groups. pT217 Tau levels of Amyloid (+) and Amyloid (−) groups were significantly different in all experiments and each p value was shown in each figure. Correlation between pT217 Tau and pT181 Tau was analyzed by Pearson correlation test. Pearson r valuc and p value were shown in FIG. 33.

### Example 13: pT217 Tau SIMOA® Assay for Plasma Samples

Reagents were same as the above pT217 Tau SIMOA® assay for CSF samples in Example 12. The assay was carried out manually. The 25 microliters of Beads coated with the test pT217 Tau recombinant antibody: 5F6-4D1, 100 microliters of 2-fold diluted plasma samples (PrecisionMed) with Tau Sample Diluent (103847, Quanterix), and 20 microliters of Detector was mixed by 800 rpm at 4 degrees Celsius for 4 hours. After this first reaction, the plate was washed with Wash Buffer A (103078, Quanterix) twice on plate washer (405TSRVS, Biotech with software of Quanterix). 100 microliters of 150 pM SBG solution (SBG concentrate was diluted with SBG Diluent) (101361, SIMOA® Enzyme and Substrate Kit, Quanterix) was added to the plate. The sample was mixed on SIMOA® plate shaker (102899, Quanterix) by 800 rpm at room temperature for 10 minutes. After the second reaction, the plate was washed with Wash Buffer A (103078, Quanterix) twice on plate washer (405TSRVS, Agilent, US with software of Quanterix). Then, the plate was washed with Wash Buffer B (103079, Quanterix) twice on plate washer (405TSRVS, Biotech with software of Quanterix). The plate after wash was set on SIMOA® SR-X analyzer (102917 Quanterix). The sample AEB (SIMOA® signal count) was transferred to the concentration compare to the AEB of calibrator sample (Tau-441 DYRKIA phosphorylated, T08-50RN, SignalChem Biotech).

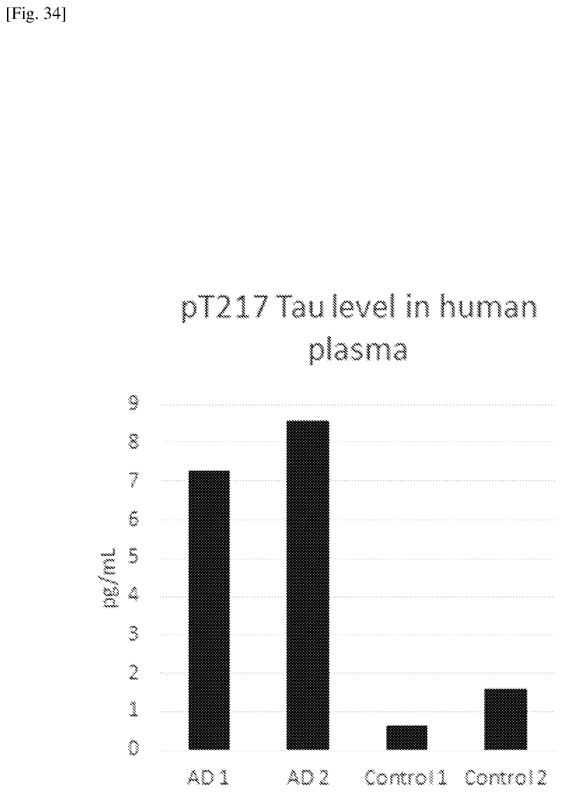

pT217 Tau concentration was measured for two AD plasma and two control plasma samples by this assay. FIG. 34 shows the results of pT217 Tau SIMOA® assay using the 5F6-4D1 antibody for plasma samples.

### Example 14: Use of the 5F6-4D1 Antibody to Detect Changes in pT217 Tau Levels in an in vivo Preclinical Model of Tau Deposition and Transmission

Tau protein has been hypothesized to form aggregated seeds that can initiate the pathological process in AD and other tauopathies. Furthermore, tau pathology spreads throughout the brain through synaptically connected pathways (de Vos et al., “Synaptic Tau Seeding Precedes Tau Pathology in Human Alzheimer's Disease Brain”, Front Neurosci. 2018;12: 267). This has been demonstrated preclinically by injecting tau seeds into specific brain regions of transgenic mice and then analyzing the presence of pathological tau in areas distal to the injection site (Narasimhan et al., “Pathological Tau Strains from Human Brains Recapitulate the Diversity of Tauopathies in Nontransgenic Mouse Brain”, J. Neurosci. 2017, vol. 37 (47) 11406-11423). In the example presented here, we injected material extracted as tau seeds from human AD brain into the left hippocampus of hTau mice which overexpress all 6 isoforms of human wild type tau (Andorfer et al., “Hyperphosphorylation and aggregation of tau in mice expressing normal human tau isoforms” 2003, vol. 86(3), 582-590). The resulting insoluble tau deposited near the injection site (ipsilateral hippocampus) as well as a distal arca (contralateral hippocampus on the right side) was analyzed at different timepoints (1, 6 and 12 weeks) following seed injection and demonstrated to be derived from the hTau mouse rather than residual material from the injected human extract. Levels of pT217 tau in the insoluble fractions of mouse tissue were evaluated using the 5F6-4D1 antibody described herein. Total tau was detected using the rabbit polyclonal antibody, K9JA (DAKO, catalogue number A0024).

Initial tau seed material was obtained from approximately 2g of fresh-frozen frontal cortex tissue from a Braak stage VI AD patient brain (Queen Square Brain Bank, London). The tissue piece was homogenized using a Tissue Ruptor (Qiagen) in 10× volume per tissue weight (v/w; 1 mL per 100 mg of tissue) Buffer A: 10 mM Tris-HCI pH7.5, 0.4 M NaCl and 11% sucrose. Homogenate was then centrifuged at 20,000 g for 20 minutes at 4° C. The resulting supernatant was retained, and the pellet was rehomogenized in 5× v/w (0.5 mL per 100 mg of starting tissue) Buffer A. Following an identical centrifugation step as before, supernatants were combined. Sarkosyl detergent (Sigma) was added to the combined supernatants to a final concentration of 1% and the mixture then agitated at room temperature for 1 hour. The sample was then split equally and centrifuged for a further 1 hour at 100,000 g and 4° C. in a 50.2 Ti rotor (Beckman). Supernatants were removed and pellets resuspended in ice-cold sterile PBS at a ratio of 0.2 mL per gram of starting material. Resuspended samples were pooled and left overnight in the fridge before aliquoting, snap freezing on dry ice and then stored at −80° C. as the ‘AD insoluble fraction’ containing tau seeds for use in the subsequent in vivo study. Presence of tau in the AD insoluble fraction was verified by western blotting.

To determine how levels of pT217 tau change over time in an in vivo model of tau deposition and spreading, hTau mice or KO littermate controls were injected with the ‘AD insoluble fraction’ described above (containing tau seed) or PBS. The KO littermate control animals were used to determine whether any insoluble pT217 tau measured was derived from the mouse or carried over from the original AD insoluble fraction seed injection material.

All mice were bred, and in vivo experimentation performed by QPS, Austria. Briefly, 10-month-old hTau transgenic mice or their KO littermate controls were injected with 3 microliters of AD insoluble fraction or vehicle (PBS) directly into the left hippocampus using the following coordinates from Bregma: anterior/posterior −1.8 mm; midline +1.4 mm (left); dorsoventral 2.1 mm. Brain tissue was collected at 1, 6, or 12 weeks following seed injection. This was performed by deeply anaesthetizing cach animal with 600 mg/kg Pentobarbital and then transcardially perfusing them with 0.9% saline. Brains were removed and hemisected before being further dissected into different brain regions which were snap frozen on dry ice and stored at -80° C. until fractionation.

For the fractionation, dissected brain tissues were sonicated in 19 v/w (1.9 mL per 100 mg) of RIPA buffer: 50 mM Tris-HCl (pH7.5), 5 mM EDTA, 1 mM EGTA, 1% NP-40 Alternative (EMD Millipore), 0.25% Sodium Deoxycholate (Bio world), 0.1 M NaCl, 0.5 mM PMSF (Sigma Aldrich), 1× PhosSTOP™M (Roche), and 1× Complete EDTA(−) (Roche). Homogenates were then centrifuged at 163,000 g at 4° C. for 25 minutes and the resulting pellet resuspended in 10× v/w (1 mL per 100 mg) of buffer containing 10 mM Tris-HCl (pH7.5), 0.5 M NaCl, 1 mM EGTA, 10% sucrose (Wako Pure Chemical), and 1% sarkosyl. Samples were then sonicated, incubated at 37° C. for 60 minutes, and then centrifuged again at 163,000 g at 4° C. for a further 25 minutes. Finally, 10 volumes of PBS were added to the pellet which was also sonicated. This formed the sarkosyl-insoluble fraction from the mouse brains.

The amount of Tau protein in each sarkosyl-insoluble fraction was quantified by Western blot analysis. Sarkosyl-insoluble fractions were solubilized in NuPAGE(R) LDS sample buffer and NuPAGE® sample reducing agent (both ThermoFisher), heated at 95° C. for 10 minutes, and denatured proteins separated using 4-12% Bis-Tris NuPAGE® gels (ThermoFisher). To aid quantitation of total tau, known amounts of non-phosphorylated recombinant 2N4R tau (SignalChem) standards were also resolved on each gel containing the fractionated tissue samples. Proteins were transferred to nitrocellulose membranes (GE Healthcare) and blots were blocked in blocking buffer: 50% Intercept Blocking Buffer (Licor), 50% TBS containing 0.1% Tween-20 (TBS-T). To detect pT217 tau or total tau, membranes were probed overnight at 4° C. with the 5F6-4D1 or K9JA antibody respectively that were diluted to 1 microgram/mL in blocking buffer. Blots were washed in TBS-T for 40 minutes and then incubated for a further 1 hour at room temperature with either a goat anti-mouse (for 5F6-4D1) or antirabbit (for K9JA) IgG conjugated to IRDye 680RD or IRDye 800CW, respectively (Licor). Secondary antibodies were removed, and blots were washed for another 40 minutes in TBS-T before scanning on an Odyssey CLX imager (Licor). Quantification was performed by band analysis using the Image Studio software (Licor).

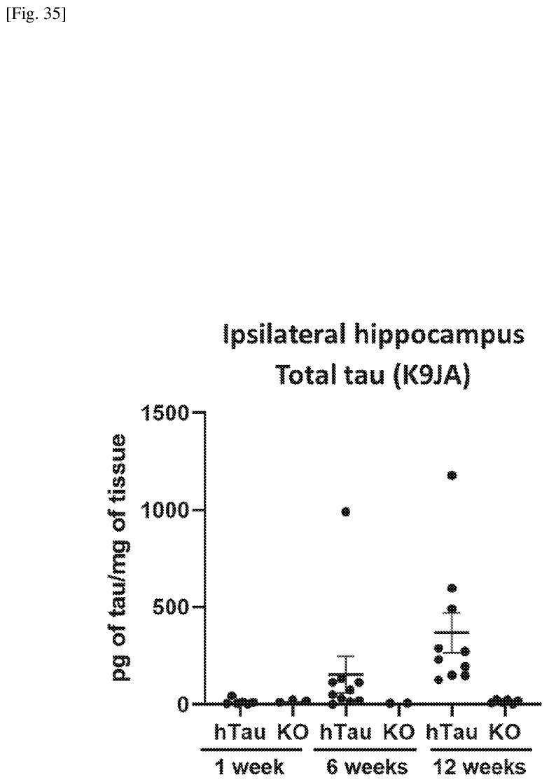

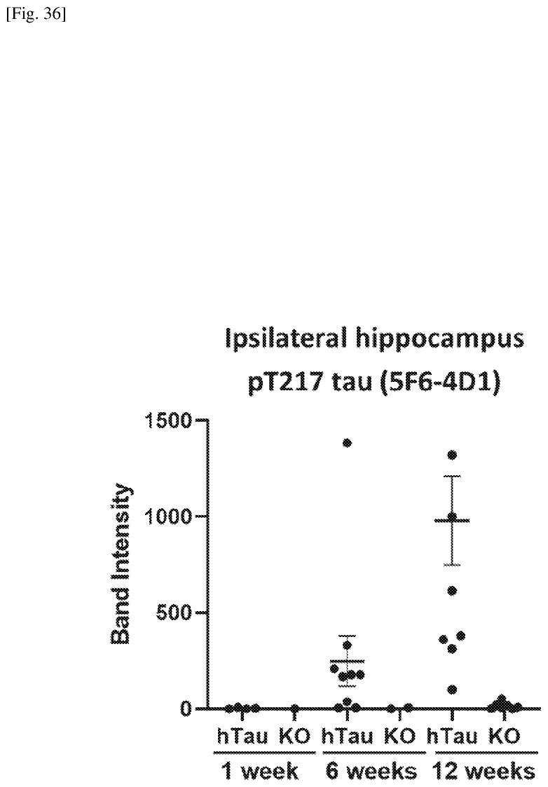

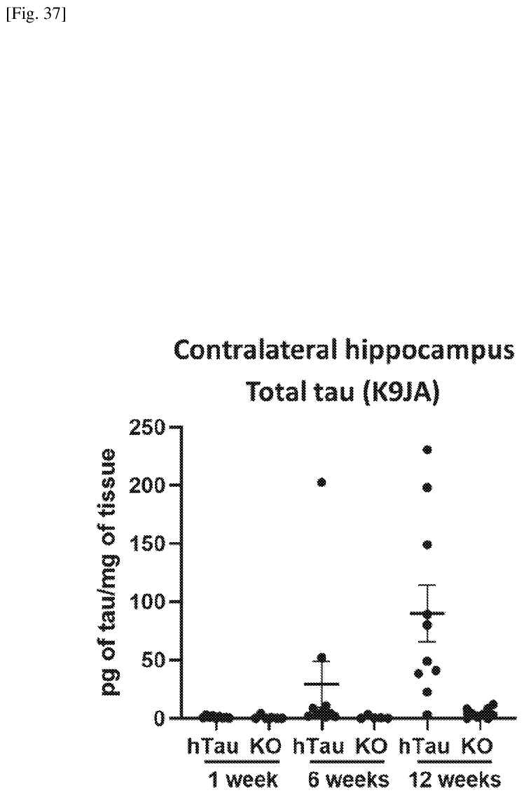

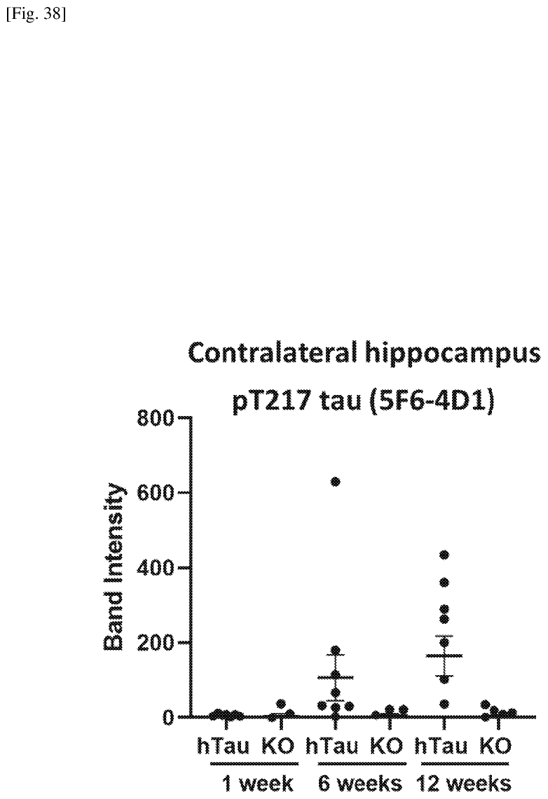

For both the ipsilateral and contralateral sides of the hippocampus, an age-dependent increase in both total tau and pT217 tau was observed in the hTau mice as detected by the K9JA and 5F6-4D1 antibodies (FIG. 35-38). Also, no signal was observed in any of the seed-injected KO littermate controls demonstrating that the insoluble tau observed following seed injection in the hTau mice is derived from expression of the transgenc. Finally, transgenic or KO animals injected with PBS instead of tau seed did not show any detectable levels of tau or pT217 tau following brain fractionation.

1. An anti-pT217 Tau antibody or a pT217 Tau-binding fragment thereof, wherein the anti-pT217 Tau antibody comprises a heavy chain and a light chain, further wherein:
(a) the heavy chain comprises a heavy chain CDR1 consisting of the amino acid sequence shown in SEQ ID NO: 1: a heavy chain CDR2 consisting of the amino acid sequence shown in SEQ ID NO: 2: and a heavy chain CDR3 consisting of the amino acid sequence shown in SEQ ID NO: 3: and the light chain comprises a light chain CDR1 consisting of the amino acid sequence shown in SEQ ID NO: 6: a light chain CDR2 consisting of the amino acid sequence shown in SEQ ID NO: 7: and a light chain CDR3 consisting of the amino acid sequence shown in SEQ ID NO: 8:
(b) the heavy chain comprises a heavy chain CDR1 consisting of the amino acid sequence shown in SEQ ID NO: 1: a heavy chain CDR2 consisting of the amino acid sequence shown in SEQ ID NO: 11: and a heavy chain CDR3 consisting of the amino acid sequence shown in SEQ ID NO: 3: and the light chain comprises a light chain CDR1 consisting of the amino acid sequence shown in SEQ ID NO: 6; a light chain CDR2 consisting of the amino acid sequence shown in SEQ ID NO: 7: and a light chain CDR3 consisting of the amino acid sequence shown in SEQ ID NO: 14:
(c) the heavy chain comprises a heavy chain CDR1 consisting of the amino acid sequence shown in SEQ ID NO: 17: a heavy chain CDR2 consisting of the amino acid sequence shown in SEQ ID NO: 18: and a heavy chain CDR3 consisting of the amino acid sequence shown in SEQ ID NO: 19; and the light chain comprises a light chain CDRI consisting of the amino acid sequence shown in SEQ ID NO: 6: a light chain CDR2 consisting of the amino acid sequence shown in SEQ ID NO: 22: and a light chain CDR3 consisting of the amino acid sequence shown in SEQ ID NO: 8:
(d) the heavy chain comprises a heavy chain CDR1 consisting of the amino acid sequence shown in SEQ ID NO: 1: a heavy chain CDR2 consisting of the amino acid sequence shown in SEQ ID NO: 25: and a heavy chain CDR3 consisting of the amino acid sequence shown in SEQ ID NO: 19; and the light chain comprises a light chain CDR1 consisting of the amino acid sequence shown in SEQ ID NO: 28: a light chain CDR2 consisting of the amino acid sequence shown in SEQ ID NO: 29: and a light chain CDR3 consisting of the amino acid sequence shown in SEQ ID NO: 8: or
(e) the heavy chain comprises a heavy chain CDR1 consisting of the amino acid sequence shown in SEQ ID NO: 1: a heavy chain CDR2 consisting of the amino acid sequence shown in SEQ ID NO: 32: and a heavy chain CDR3 consisting of the amino acid sequence shown in SEQ ID NO: 19; and the light chain comprises a light chain CDR1 consisting of the amino acid sequence shown in SEQ ID NO: 6; a light chain CDR2 consisting of the amino acid sequence shown in SEQ ID NO: 29; and a light chain CDR3 consisting of the amino acid sequence shown in SEQ ID NO: 8.

2.-6. (canceled)

7. The anti-pT217 Tau antibody or the pT217 Tau-binding fragment thereof according to claim 1, wherein the anti-pT217 Tau antibody comprises:
(f) a heavy chain variable domain consisting of the amino acid sequence shown in SEQ ID NO: 4, and a light chain variable domain consisting of the amino acid sequence shown in SEQ ID NO: 9;
(g) a heavy chain variable domain consisting of the amino acid sequence shown in SEQ ID NO: 12, and a light chain variable domain consisting of the amino acid sequence shown in SEQ ID NO: 15:
(h) a heavy chain variable domain consisting of the amino acid sequence shown in SEQ ID NO: 20, and a light chain variable domain consisting of the amino acid sequence shown in SEQ ID NO: 23;
(i) a heavy chain variable domain consisting of the amino acid sequence shown in SEQ ID NO: 26, and a light chain variable domain consisting of the amino acid sequence shown in SEQ ID NO: 30;
(j) a heavy chain variable domain consisting of the amino acid sequence shown in SEQ ID NO: 33, and a light chain variable domain consisting of the amino acid sequence shown in SEQ ID NO: 35; or
(k) a heavy chain variable domain consisting of the amino acid sequence shown in SEQ ID NO: 12, and a light chain variable domain consisting of the amino acid sequence shown in SEQ ID NO: 63.

8. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 4, and a light chain variable domain consisting of the amino acid sequence shown in SEQ ID NO: 9.

9. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 12, and a light chain variable domain consisting of the amino acid sequence shown in SEQ ID NO: 15.

10. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 20, and a light chain variable domain consisting of the amino acid sequence shown in SEQ ID NO: 23.

11. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 26, and a light chain variable domain consisting of the amino acid sequence shown in SEQ ID NO: 30.

12. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 33, and a light chain variable domain consisting of the amino acid sequence shown in SEQ ID NO: 35.

13. The anti-pT217 Tau antibody or the pT217 Tau-binding fragment thereof according to claim 7, wherein the anti-pT217 Tau antibody comprises a heavy chain variable domain consisting of the amino acid sequence shown in SEQ ID NO: 12, and a light chain variable domain consisting of the amino acid sequence shown in SEQ ID NO: 63.

14. A method for detecting the presence or amount of pT217 Tau in a biological sample, comprising:
contacting the biological sample with the anti-pT217 Tau antibody or the pT217 Tau-binding fragment thereof according to claim 1; and
detecting the presence or amount of complex formed between the anti-pT217 Tau antibody or pT217 Tau-binding fragment and pT217 Tau in the biological sample.

15. A method for evaluating amyloid beta accumulation in a subject, comprising:
contacting a biological sample from the subject with the anti-pT217 Tau antibody or the pT217 Tau-binding fragment thereof according to claim 1; and
detecting the presence or amount of complex formed between the anti-pT217 Tau antibody or pT217 Tau-binding fragment and pT217 Tau in the biological sample.

16. The method according to claim 15, wherein the method further comprises comparing the amount of complex in the biological sample to an amount of complex formed in a control biological sample.

17. The method according to claim 14, wherein the biological sample is blood, serum, plasma, or cerebral spinal fluid.

18. The method according to claim 14, wherein the biological sample is obtained from a subject that suffers from or is at risk of suffering from Alzheimer's disease or mild cognitive impairment due to Alzheimer's disease.

19.-20. (canceled)

21. The method according to claim 15, wherein the biological sample is blood, serum, plasma, or cerebral spinal fluid.

22. The method according to claim 15, wherein the biological sample is obtained from a subject that suffers from or is at risk of suffering from Alzheimer's disease or mild cognitive impairment due to Alzheimer's disease.

23. A kit for detecting the presence or amount of pT217 Tau, comprising the anti-pT217 Tau antibody or the pT217 Tau-binding fragment thereof according to claim 1.

24. A kit for evaluating amyloid beta accumulation, comprising the anti-pT217 Tau antibody or the pT217 Tau-binding fragment thereof according to claim 1.


SEQUENCE LISTING (105 sequences)

SEQ ID NO: 1 - protein, 5 aa, Mus musculus
DYGMH

SEQ ID NO: 2 - protein, 17 aa, Mus musculus
YMSSGSSTIYYVDTVKG

SEQ ID NO: 3 - protein, 4 aa, Mus musculus
RLSI

SEQ ID NO: 4 - protein, 113 aa, Mus musculus
EVQLVESGGGLVKPGGSLKLSCAASGFTLSDYGMHWVRQAPEKGLEWIAYMSSGSSTIYYVDTVKGRFTISRDNAKNTLFLQMTSLRSEDTAMYYCARRLSIWGTGTTVTVSS

SEQ ID NO: 5 - protein, 449 aa, Mus musculus
EVQLVESGGGLVKPGGSLKLSCAASGFTLSDYGMHWVRQAPEKGLEWIAYMSSGSSTIYYVDTVKGRFTISRDNAKNTLFLQMTSLRSEDTAMYYCARRLSIWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 6 - protein, 10 aa, Mus musculus
SASSSVSSMY

SEQ ID NO: 7 - protein, 7 aa, Mus musculus
DTSNLAS

SEQ ID NO: 8 - protein, 9 aa, Mus musculus
QQWSSYPLT

SEQ ID NO: 9 - protein, 106 aa, Mus musculus
QIVLTQSPAIMSASPGEKVTMTCSASSSVSSMYWYHQKPGSSPRLLIYDTSNLASGVPVRFSGSGSGTSYSLTISRMEAEDAATYYCQQWSSYPLTFGAGTKLELK

SEQ ID NO: 10 - protein, 213 aa, Mus musculus
QIVLTQSPAIMSASPGEKVTMTCSASSSVSSMYWYHQKPGSSPRLLIYDTSNLASGVPVRFSGSGSGTSYSLTISRMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 17 aa, Mus musculus
YISSGSSTIYYVDTVKG

SEQ ID NO: 12 - protein, 113 aa, Mus musculus
EVQLVESGGGLVKPGGSLKLSCAASGFTVSDYGMHWVRQAPEKGLEWIAYISSGSSTIYYVDTVKGRFTISRDNAKNTLFLQMTSLRSEDTAMYYCARRLSIWGTGTTVTVSS

SEQ ID NO: 13 - protein, 449 aa, Mus musculus
EVQLVESGGGLVKPGGSLKLSCAASGFTVSDYGMHWVRQAPEKGLEWIAYISSGSSTIYYVDTVKGRFTISRDNAKNTLFLQMTSLRSEDTAMYYCARRLSIWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 14 - protein, 9 aa, Mus musculus
LQWSSYPLT

SEQ ID NO: 15 - protein, 106 aa, Mus musculus
QIVLTQSPAIMSACPGEKVTMTCSASSSVSSMYWYHQKPGSSPRLLIYDTSNLASGVPVRFSGSGSGTSYSLTISRMEAEDAATYYCLQWSSYPLTFGAGTKLELK

SEQ ID NO: 16 - protein, 213 aa, Mus musculus
QIVLTQSPAIMSACPGEKVTMTCSASSSVSSMYWYHQKPGSSPRLLIYDTSNLASGVPVRFSGSGSGTSYSLTISRMEAEDAATYYCLQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 5 aa, Mus musculus
DYGIH

SEQ ID NO: 18 - protein, 17 aa, Mus musculus
YISSGSNTIYYIDTVKG

SEQ ID NO: 19 - protein, 4 aa, Mus musculus
RMAY

SEQ ID NO: 20 - protein, 113 aa, Mus musculus
EVQLVESGGGLVKPGGSLKLSCVASGFSVSDYGIHWVRQAPEKGLEWVAYISSGSNTIYYIDTVKGRFTISRDNAKNTLFLQMTSLRSEDTAMYYCARRMAYWGQGTLVTVSA

SEQ ID NO: 21 - protein, 449 aa, Mus musculus
EVQLVESGGGLVKPGGSLKLSCVASGFSVSDYGIHWVRQAPEKGLEWVAYISSGSNTIYYIDTVKGRFTISRDNAKNTLFLQMTSLRSEDTAMYYCARRMAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 22 - protein, 7 aa, Mus musculus
ATFNLAS

SEQ ID NO: 23 - protein, 106 aa, Mus musculus
QIVLTQSPAIMSTSPGEKVTMTCSASSSVSSMYWYQQKPGSSPRLLIYATFNLASGVPVRFSGSGSGTSYSLTISRMEAEDAATYYCQQWSSYPLTFGAGTKLEVK

SEQ ID NO: 24 - protein, 213 aa, Mus musculus
QIVLTQSPAIMSTSPGEKVTMTCSASSSVSSMYWYQQKPGSSPRLLIYATFNLASGVPVRFSGSGSGTSYSLTISRMEAEDAATYYCQQWSSYPLTFGAGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 17 aa, Mus musculus
YISSGDSTIYYADTVKG

SEQ ID NO: 26 - protein, 113 aa, Mus musculus
EVQLVESGGGLVKPGGSLKLSCAASGFTLSDYGMHWVRQAPEKGLEWFAYISSGDSTIYYADTVKGRFTISRDNAKNTLFLQMTSLRSEDTAMYYCARRMAYWGQGTLVTVSA

SEQ ID NO: 27 - protein, 449 aa, Mus musculus
EVQLVESGGGLVKPGGSLKLSCAASGFTLSDYGMHWVRQAPEKGLEWFAYISSGDSTIYYADTVKGRFTISRDNAKNTLFLQMTSLRSEDTAMYYCARRMAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 28 - protein, 10 aa, Mus musculus
SASSSVTYMY

SEQ ID NO: 29 - protein, 7 aa, Mus musculus
ATSNLAS

SEQ ID NO: 30 - protein, 106 aa, Mus musculus
QIVLTQSPAIMSASPGEKVTMTCSASSSVTYMYWYQQKPGSSPRLLIYATSNLASGVPVRFSGSGSGTSYSLTISRMEAEDAATYYCQQWSSYPLTFGAGTKLELK

SEQ ID NO: 31 - protein, 213 aa, Mus musculus
QIVLTQSPAIMSASPGEKVTMTCSASSSVTYMYWYQQKPGSSPRLLIYATSNLASGVPVRFSGSGSGTSYSLTISRMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 17 aa, Mus musculus
YISSGSSTIYYADTVKG

SEQ ID NO: 33 - protein, 113 aa, Mus musculus
EVQLVESGGGLVKPGGSLKLSCSASGFTISDYGMHWVRQAPEKGLEWFAYISSGSSTIYYADTVKGRFTISRDNAKNTLFLQMTSLRSEDTAMYYCARRMAYWGQGTLVTVSA

SEQ ID NO: 34 - protein, 449 aa, Mus musculus
EVQLVESGGGLVKPGGSLKLSCSASGFTISDYGMHWVRQAPEKGLEWFAYISSGSSTIYYADTVKGRFTISRDNAKNTLFLQMTSLRSEDTAMYYCARRMAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 35 - protein, 106 aa, Mus musculus
QIVLTQSPAIMSASPGEKVTMTCSASSSVSSMYWYQQKPGSSPRLLIYATSNLASGVPVRFSGSGSGTSYSLTISRMEAEDAATYYCQQWSSYPLTFGAGTKLELK

SEQ ID NO: 36 - protein, 213 aa, Mus musculus
QIVLTQSPAIMSASPGEKVTMTCSASSSVSSMYWYQQKPGSSPRLLIYATSNLASGVPVRFSGSGSGTSYSLTISRMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - DNA, 15 bp, Mus musculus
gactacggcatgcac

SEQ ID NO: 38 - DNA, 51 bp, Mus musculus
tatatcagctccggctctagcaccatctactatgtggacacagtgaagggc

SEQ ID NO: 39 - DNA, 12 bp, Mus musculus
agactgtccatc

SEQ ID NO: 40 - DNA, 339 bp, Mus musculus
gaggtgcagctggtggagtccggaggaggactggtgaagccaggaggctccctgaagctgtcttgcgccgccagcggctttaccgtgagcgactacggcatgcactgggtgaggcaggcacctgagaagggactggagtggatcgcctatatcagctccggctctagcaccatctactatgtggacacagtgaagggcaggttcaccatctcccgcgataacgccaagaatacactgtttctgcagatgaccagcctgcgctccgaggacacagccatgtactattgtgcacggagactgtccatctggggaaccggaaccacagtgacagtgtcctct

SEQ ID NO: 41 - DNA, 1347 bp, Mus musculus
gaggtgcagctggtggagtccggaggaggactggtgaagccaggaggctccctgaagctgtcttgcgccgccagcggctttaccgtgagcgactacggcatgcactgggtgaggcaggcacctgagaagggactggagtggatcgcctatatcagctccggctctagcaccatctactatgtggacacagtgaagggcaggttcaccatctcccgcgataacgccaagaatacactgtttctgcagatgaccagcctgcgctccgaggacacagccatgtactattgtgcacggagactgtccatctggggaaccggaaccacagtgacagtgtcctctgccaagaccacaccacctagcgtgtaccctctggcaccaggatgcggcgataccacaggaagctccgtgaccctgggctgtctggtgaagggctatttcccagagagcgtgaccgtgacatggaacagcggctccctgtctagctccgtgcacacatttcccgccctgctgcagtccggactgtacaccatgtctagctccgtgaccgtgccatctagcacatggccctcccagaccgtgacatgctctgtggcccaccctgcctcctctaccacagtggacaagaagctggagccctccggccctatctctaccatcaatccctgcccaccctgtaaggagtgccacaagtgtccagcacctaacctggagggaggacctagcgtgttcatctttcctccaaatatcaaggatgtgctgatgatcagcctgacccctaaggtgacatgcgtggtggtggacgtgagcgaggacgatccagatgtgcggatctcctggttcgtgaacaatgtggaggtgcacacagcccagacccagacacaccgggaggactacaacagcaccatcagagtggtgtccgccctgccaatccagcaccaggactggatgtccggcaaggagtttaagtgcaaggtgaacaataaggatctgccatctcccatcgagaggaccatcagcaagatcaagggactggtgcgcgcaccacaggtgtacatcctgccacctccagcagagcagctgagcagaaaggacgtgtccctgacatgtctggtggtgggcttcaatcctggcgatatctctgtggagtggaccagcaacggccacacagaggagaattataaggataccgccccagtgctggactctgatggcagctacttcatctattccaagctggacatcaagacatctaagtgggagaagaccgattcttttagctgcaacgtgcggcacgagggcctgaagaattactatctgaagaagaccatctccagatctcccggcaag

SEQ ID NO: 42 - DNA, 30 bp, Mus musculus
agcgccagctcctctgtgagctccatgtac

SEQ ID NO: 43 - DNA, 21 bp, Mus musculus
gatacatctaacctggcaagc

SEQ ID NO: 44 - DNA, 27 bp, Mus musculus
ctgcagtggtcctcttaccccctgaca

SEQ ID NO: 45 - DNA, 318 bp, Mus musculus
cagatcgtgctgacccagagccctgcaatcatgtccgcctgcccaggagagaaggtgaccatgacatgtagcgccagctcctctgtgagctccatgtactggtatcaccagaagccaggctctagcccccggctgctgatctacgatacatctaacctggcaagcggagtgcccgtgagattctccggctctggcagcggcacatcctattctctgaccatctccaggatggaggcagaggacgcagcaacctactattgcctgcagtggtcctcttaccccctgacattcggcgccggcaccaagctggagctgaag

SEQ ID NO: 46 - DNA, 639 bp, Mus musculus
cagatcgtgctgacccagagccctgcaatcatgtccgcctgcccaggagagaaggtgaccatgacatgtagcgccagctcctctgtgagctccatgtactggtatcaccagaagccaggctctagcccccggctgctgatctacgatacatctaacctggcaagcggagtgcccgtgagattctccggctctggcagcggcacatcctattctctgaccatctccaggatggaggcagaggacgcagcaacctactattgcctgcagtggtcctcttaccccctgacattcggcgccggcaccaagctggagctgaagagagccgatgccgcccctacagtgagcatctttccccctagctccgagcagctgacctccggaggagcatctgtggtgtgcttcctgaacaacttctacccaaaggacatcaacgtgaagtggaagatcgatggctctgagaggcagaacggcgtgctgaatagctggacagaccaggattctaaggacagcacctattccatgtctagcaccctgacactgaccaaggatgagtacgagcgccacaattcctatacatgcgaggccacccacaagacaagcacctcccccatcgtgaagtcttttaaccggaatgagtgt

SEQ ID NO: 47 - DNA, 15 bp, Mus musculus
gactacggcatccac

SEQ ID NO: 48 - DNA, 51 bp, Mus musculus
tatatcagctccggcagcaacaccatctactatatcgacacagtgaagggc

SEQ ID NO: 49 - DNA, 12 bp, Mus musculus
agaatggcatac

SEQ ID NO: 50 - DNA, 339 bp, Mus musculus
gaggtgcagctggtggagtctggaggaggactggtgaagccaggaggctctctgaagctgagctgcgtggcctccggcttttctgtgagcgactacggcatccactgggtgaggcaggcacctgagaagggactggagtgggtggcctatatcagctccggcagcaacaccatctactatatcgacacagtgaagggcaggttcaccatctcccgcgataacgccaagaatacactgtttctgcagatgacctccctgcgctctgaggacacagccatgtactattgtgcaaggagaatggcatactggggacagggcaccctggtgacagtgtccgcc

SEQ ID NO: 51 - DNA, 1347 bp, Mus musculus
gaggtgcagctggtggagtctggaggaggactggtgaagccaggaggctctctgaagctgagctgcgtggcctccggcttttctgtgagcgactacggcatccactgggtgaggcaggcacctgagaagggactggagtgggtggcctatatcagctccggcagcaacaccatctactatatcgacacagtgaagggcaggttcaccatctcccgcgataacgccaagaatacactgtttctgcagatgacctccctgcgctctgaggacacagccatgtactattgtgcaaggagaatggcatactggggacagggcaccctggtgacagtgtccgccgccaagaccacaccacctagcgtgtaccctctggcaccaggatgcggcgataccacaggatctagcgtgaccctgggctgtctggtgaagggctacttcccagagagcgtgaccgtgacatggaactccggctctctgtcctctagcgtgcacacatttcccgccctgctgcagtccggcctgtataccatgtcctctagcgtgaccgtgccatcctctacatggccctctcagaccgtgacatgcagcgtggcccaccctgccagctccaccacagtggacaagaagctggagccctctggccctatcagcaccatcaatccctgcccaccctgtaaggagtgccacaagtgtccagcacctaacctggagggaggacctagcgtgttcatctttcctccaaatatcaaggatgtgctgatgatctccctgacccctaaggtgacatgcgtggtggtggacgtgagcgaggacgatccagatgtgcggatctcctggttcgtgaacaatgtggaggtgcacacagcccagacccagacacaccgggaggactacaactccaccatcagagtggtgtctgccctgccaatccagcaccaggactggatgagcggcaaggagtttaagtgcaaggtgaacaataaggatctgccaagccccatcgagaggaccatctccaagatcaagggactggtgcgcgcaccacaggtgtacatcctgccacctccagcagagcagctgtccagaaaggacgtgtctctgacatgtctggtggtgggcttcaatcctggcgatatcagcgtggagtggacctccaacggccacacagaggagaattataaggataccgccccagtgctggacagcgatggctcctacttcatctattctaagctggacatcaagacaagcaagtgggagaagaccgatagcttttcctgcaacgtgcggcacgagggcctgaagaattactatctgaagaagaccatctctagaagccccggcaag

SEQ ID NO: 52 - DNA, 21 bp, Mus musculus
gccacattcaacctggcatcc

SEQ ID NO: 53 - DNA, 27 bp, Mus musculus
cagcagtggtcctcttaccccctgaca

SEQ ID NO: 54 - DNA, 318 bp, Mus musculus
cagatcgtgctgacacagtctcctgccatcatgtccacctctccaggcgagaaggtgaccatgacatgtagcgccagctcctctgtgagctccatgtactggtatcagcagaagccaggctctagcccccggctgctgatctacgccacattcaacctggcatccggagtgcccgtgcggttcagcggctccggctctggcacaagctattccctgaccatcagcaggatggaggcagaggacgcagcaacctactattgccagcagtggtcctcttaccccctgacattcggcgccggcaccaagctggaggtgaag

SEQ ID NO: 55 - DNA, 639 bp, Mus musculus
cagatcgtgctgacacagtctcctgccatcatgtccacctctccaggcgagaaggtgaccatgacatgtagcgccagctcctctgtgagctccatgtactggtatcagcagaagccaggctctagcccccggctgctgatctacgccacattcaacctggcatccggagtgcccgtgcggttcagcggctccggctctggcacaagctattccctgaccatcagcaggatggaggcagaggacgcagcaacctactattgccagcagtggtcctcttaccccctgacattcggcgccggcaccaagctggaggtgaagagagccgatgccgcccctacagtgagcatctttccccctagctccgagcagctgacctccggaggagcatctgtggtgtgcttcctgaacaacttctacccaaaggacatcaacgtgaagtggaagatcgatggcagcgagaggcagaacggcgtgctgaacagctggacagaccaggattccaaggactctacctatagcatgtctagcaccctgacactgaccaaggatgagtacgagcgccacaattcctatacatgcgaggccacccacaagacatctaccagccccatcgtgaagtctttcaaccggaatgagtgt

SEQ ID NO: 56 - DNA, 51 bp, Mus musculus
tatatcagctccggctctagcaccatctactatgccgacacagtgaagggc

SEQ ID NO: 57 - DNA, 339 bp, Mus musculus
gaggtgcagctggtggagtctggaggaggactggtgaagccaggaggctctctgaagctgtcctgctctgccagcggcttcaccatctccgactacggcatgcactgggtgaggcaggcacctgagaagggactggagtggtttgcctatatcagctccggctctagcaccatctactatgccgacacagtgaagggcaggttcaccatcagccgcgataacgccaagaatacactgtttctgcagatgacctccctgcgctctgaggacacagccatgtactattgtgcaaggagaatggcatactggggacagggcaccctggtgacagtgtccgcc

SEQ ID NO: 58 - DNA, 1347 bp, Mus musculus
gaggtgcagctggtggagtctggaggaggactggtgaagccaggaggctctctgaagctgtcctgctctgccagcggcttcaccatctccgactacggcatgcactgggtgaggcaggcacctgagaagggactggagtggtttgcctatatcagctccggctctagcaccatctactatgccgacacagtgaagggcaggttcaccatcagccgcgataacgccaagaatacactgtttctgcagatgacctccctgcgctctgaggacacagccatgtactattgtgcaaggagaatggcatactggggacagggcaccctggtgacagtgtccgccgccaagaccacaccacctagcgtgtaccctctggcaccaggatgcggcgataccacaggatcctctgtgaccctgggctgtctggtgaagggctacttcccagagagcgtgaccgtgacatggaactccggctctctgagctcctctgtgcacacatttcccgccctgctgcagtccggcctgtataccatgagctcctctgtgaccgtgccaagctccacatggccctctcagaccgtgacatgcagcgtggcccaccctgcctctagcaccacagtggacaagaagctggagccctctggccctatcagcaccatcaatccctgcccaccctgtaaggagtgccacaagtgtccagcacctaacctggagggaggacctagcgtgttcatctttcctccaaatatcaaggatgtgctgatgatctccctgacccctaaggtgacatgcgtggtggtggacgtgagcgaggacgatccagatgtgcggatctcctggttcgtgaacaatgtggaggtgcacacagcccagacccagacacaccgggaggactacaactccaccatcagagtggtgtctgccctgccaatccagcaccaggactggatgagcggcaaggagtttaagtgcaaggtgaacaataaggatctgccaagccccatcgagaggaccatctccaagatcaagggactggtgcgcgcaccacaggtgtacatcctgccacctccagcagagcagctgtccagaaaggacgtgtctctgacatgtctggtggtgggctttaatcctggcgatatcagcgtggagtggacctccaacggccacacagaggagaattataaggataccgccccagtgctggacagcgatggctcctacttcatctattctaagctggacatcaagacaagcaagtgggagaagaccgatagcttttcctgcaacgtgcggcacgagggcctgaagaattactatctgaagaagaccatctctagaagccccggcaag

SEQ ID NO: 59 - DNA, 30 bp, Mus musculus
tctgccagctcctctgtgagctccatgtac

SEQ ID NO: 60 - DNA, 21 bp, Mus musculus
gccacatccaacctggcctct

SEQ ID NO: 61 - DNA, 318 bp, Mus musculus
cagatcgtgctgacccagtcccctgccatcatgtccgcctctccaggcgagaaggtgaccatgacatgttctgccagctcctctgtgagctccatgtactggtatcagcagaagccaggctctagcccccggctgctgatctacgccacatccaacctggcctctggagtgcccgtgagattcagcggatccggatctggcacaagctattccctgaccatctccaggatggaggcagaggacgcagcaacctactattgccagcagtggtcctcttaccccctgacattcggcgccggcaccaagctggagctgaag

SEQ ID NO: 62 - DNA, 639 bp, Mus musculus
cagatcgtgctgacccagtcccctgccatcatgtccgcctctccaggcgagaaggtgaccatgacatgttctgccagctcctctgtgagctccatgtactggtatcagcagaagccaggctctagcccccggctgctgatctacgccacatccaacctggcctctggagtgcccgtgagattcagcggatccggatctggcacaagctattccctgaccatctccaggatggaggcagaggacgcagcaacctactattgccagcagtggtcctcttaccccctgacattcggcgccggcaccaagctggagctgaagagagccgatgccgcccctacagtgagcatctttccccctagctccgagcagctgaccagcggaggagcatccgtggtgtgcttcctgaacaacttctacccaaaggacatcaacgtgaagtggaagatcgatggctctgagaggcagaacggcgtgctgaatagctggacagaccaggattccaaggactctacctatagcatgtctagcaccctgacactgaccaaggatgagtacgagcgccacaattcctatacatgcgaggccacccacaagacatctaccagccccatcgtgaagagctttaaccggaatgagtgt

SEQ ID NO: 63 - protein, 106 aa, Mus musculus
QIVLTQSPAIMSASPGEKVTMTCSASSSVSSMYWYHQKPGSSPRLLIYDTSNLASGVPVRFSGSGSGTSYSLTISRMEAEDAATYYCLQWSSYPLTFGAGTKLELK

SEQ ID NO: 64 - protein, 213 aa, Mus musculus
QIVLTQSPAIMSASPGEKVTMTCSASSSVSSMYWYHQKPGSSPRLLIYDTSNLASGVPVRFSGSGSGTSYSLTISRMEAEDAATYYCLQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - DNA, 318 bp, Mus musculus
cagatcgtgctgacccagagccctgcaatcatgtccgccagcccaggagagaaggtgaccatgacatgtagcgccagctcctctgtgagctccatgtactggtatcaccagaagccaggctctagcccccggctgctgatctacgatacatctaacctggcaagcggagtgcccgtgagattctccggctctggcagcggcacatcctattctctgaccatctccaggatggaggcagaggacgcagcaacctactattgcctgcagtggtcctcttaccccctgacattcggcgccggcaccaagctggagctgaag

SEQ ID NO: 66 - DNA, 639 bp, Mus musculus
cagatcgtgctgacccagagccctgcaatcatgtccgccagcccaggagagaaggtgaccatgacatgtagcgccagctcctctgtgagctccatgtactggtatcaccagaagccaggctctagcccccggctgctgatctacgatacatctaacctggcaagcggagtgcccgtgagattctccggctctggcagcggcacatcctattctctgaccatctccaggatggaggcagaggacgcagcaacctactattgcctgcagtggtcctcttaccccctgacattcggcgccggcaccaagctggagctgaagagagccgatgccgcccctacagtgagcatctttccccctagctccgagcagctgacctccggaggagcatctgtggtgtgcttcctgaacaacttctacccaaaggacatcaacgtgaagtggaagatcgatggctctgagaggcagaacggcgtgctgaatagctggacagaccaggattctaaggacagcacctattccatgtctagcaccctgacactgaccaaggatgagtacgagcgccacaattcctatacatgcgaggccacccacaagacaagcacctcccccatcgtgaagtcttttaaccggaatgagtgt

SEQ ID NO: 67 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 68 - protein, 12 aa, Homo sapiens
  MISC_FEATURE (6)..(6): phosphorylated
TPSLPTPPTREC

SEQ ID NO: 69 - protein, 19 aa, Mus musculus
MEWSWVFLFFLSVTTGVHS

SEQ ID NO: 70 - DNA, 57 bp, Mus musculus
atggagtggagctgggtgttcctgttctttctgagcgtgaccacaggcgtgcactcc

SEQ ID NO: 71 - protein, 20 aa, Mus musculus
MSVPTQVLGLLLLWLTDARC

SEQ ID NO: 72 - DNA, 60 bp, Mus musculus
atgtccgtgcctacacaggtgctgggactgctgctgctgtggctgaccgacgccagatgc

SEQ ID NO: 73 - protein, 36 aa, Homo sapiens
  MISC_FEATURE (28)..(28): phosphorylated
KSGDRSGYSSPGSPGTPGSRSRTPSLPTPPTREPKK

SEQ ID NO: 74 - protein, 17 aa, Homo sapiens
  MISC_FEATURE (9)..(9): phosphorylated
RSRTPSLPTPPTREPKK

SEQ ID NO: 75 - protein, 17 aa, Homo sapiens
RSRTPSLPTPPTREPKK

SEQ ID NO: 76 - DNA, 57 bp, Mus musculus
atggaatggtcctgggtgttcctgttcttcctgagcgtgacaaccggcgtgcacagc

SEQ ID NO: 77 - DNA, 60 bp, Mus musculus
atgagcgtgcctacacaggtgctgggcctgctcctgctgtggctgaccgacgctagatgt

SEQ ID NO: 78 - DNA, 57 bp, Mus musculus
atggaatggagctgggtctttctgttcttcctgagcgtgacaaccggcgtgcacagc

SEQ ID NO: 79 - DNA, 60 bp, Mus musculus
atgagcgtccccacacaggtgctgggcctgctgctgctctggctgacagatgccagatgt

SEQ ID NO: 80 - DNA, 51 bp, Mus musculus
tacatgagcagcggaagctccaccatctactacgtggacacagtgaagggc

SEQ ID NO: 81 - DNA, 12 bp, Mus musculus
agactgtctatc

SEQ ID NO: 82 - DNA, 339 bp, Mus musculus
gaggtgcagctggtcgagtctggcggcggactggtgaaacctgggggctccctgaaactgagctgcgccgcctctggatttacactgagcgactacggcatgcactgggtgagacaggccccagagaagggcctggaatggatcgcctacatgagcagcggaagctccaccatctactacgtggacacagtgaagggcagattcaccatcagccgggacaacgccaagaacacactgtttctgcagatgacaagccttagatctgaggacacagctatgtattactgcgctagaagactgtctatctggggcaccggaaccaccgtgacagtgagcagc

SEQ ID NO: 83 - DNA, 1347 bp, Mus musculus
gaggtgcagctggtcgagtctggcggcggactggtgaaacctgggggctccctgaaactgagctgcgccgcctctggatttacactgagcgactacggcatgcactgggtgagacaggccccagagaagggcctggaatggatcgcctacatgagcagcggaagctccaccatctactacgtggacacagtgaagggcagattcaccatcagccgggacaacgccaagaacacactgtttctgcagatgacaagccttagatctgaggacacagctatgtattactgcgctagaagactgtctatctggggcaccggaaccaccgtgacagtgagcagcgccaagaccacccctcccagcgtctaccccctggccccaggctgcggcgacaccacaggaagcagcgtgaccctgggctgtctggtgaaaggctacttccctgaaagcgtgaccgtgacatggaacagcggcagtctcagcagctctgtccacaccttccccgctctgctgcaatctggcctctacaccatgtcatctagcgtgactgtccccagtagcacctggcctagccagacagtgacatgcagcgtggcccaccccgccagcagcaccaccgtggacaagaagctggaacctagcggtcctatctccacaatcaacccttgccctccatgtaaagagtgccacaagtgtcctgctccaaacctggaaggcggccctagcgttttcatcttccctcctaatatcaaggacgtgctgatgatcagcctgacacctaaggtgacctgtgtggtggtggatgtgtctgaggacgaccctgatgtgcgcatctcttggttcgtgaacaacgtggaagtgcataccgcccagacccagacccacagagaggattacaacagcaccattagagtggtgtccgccctgcccatccaacaccaggactggatgagcggaaaggagttcaagtgcaaggttaacaacaaggacctgcctagccccatcgagcggaccatctctaaaatcaagggcctggtgcgggccccccaggtgtacatcctgcctcctcccgccgagcagctgagcaggaaggatgtgagcctgacctgcctggtggtgggcttcaaccctggcgatatcagcgtcgagtggacctctaatggccacacggaagagaactataaggacaccgcccctgtgctggactccgacggcagctactttatctacagcaagctggatatcaagaccagcaagtgggagaagacagatagcttcagctgcaacgtgcggcacgagggcctgaagaactactacctgaagaaaaccattagcagaagcccaggcaag

SEQ ID NO: 84 - DNA, 30 bp, Mus musculus
agcgccagcagcagcgtgtctagcatgtac

SEQ ID NO: 85 - DNA, 21 bp, Mus musculus
gacacctctaatctggcctct

SEQ ID NO: 86 - DNA, 27 bp, Mus musculus
cagcagtggtccagctaccctctgacc

SEQ ID NO: 87 - DNA, 318 bp, Mus musculus
caaatcgtgctgacacagagccctgccatcatgtccgccagccccggcgagaaggtgacaatgacctgtagcgccagcagcagcgtgtctagcatgtactggtaccaccagaagcctggctcttctccaagactgctgatctacgacacctctaatctggcctctggagtccccgtgcggttcagcggctctggcagcggcacatcatatagcctgacaatcagcagaatggaagctgaagatgccgctacatactactgccagcagtggtccagctaccctctgacctttggagccggcaccaagctggaactgaag

SEQ ID NO: 88 - DNA, 639 bp, Mus musculus
caaatcgtgctgacacagagccctgccatcatgtccgccagccccggcgagaaggtgacaatgacctgtagcgccagcagcagcgtgtctagcatgtactggtaccaccagaagcctggctcttctccaagactgctgatctacgacacctctaatctggcctctggagtccccgtgcggttcagcggctctggcagcggcacatcatatagcctgacaatcagcagaatggaagctgaagatgccgctacatactactgccagcagtggtccagctaccctctgacctttggagccggcaccaagctggaactgaagcgggccgacgccgcccctaccgtgtccatcttcccacctagctctgaacagctgaccagcggaggcgcctctgttgtgtgcttcctgaacaacttctaccccaaggacatcaacgtgaagtggaagatcgacggcagcgagcggcagaacggcgtgctgaatagctggacagatcaggactccaaggatagcacctacagcatgagcagcaccctgaccctgaccaaagacgagtacgagagacacaacagctacacctgcgaggccacacacaagacctccaccagccctattgtgaagagcttcaacagaaacgagtgc

SEQ ID NO: 89 - DNA, 51 bp, Mus musculus
tacatcagctctggcgactccacaatttactacgccgacaccgtgaaggga

SEQ ID NO: 90 - DNA, 12 bp, Mus musculus
agaatggcctac

SEQ ID NO: 91 - DNA, 339 bp, Mus musculus
gaggtgcaactggttgagagcggcggtggcctggtgaaacctggcggctctctgaagctgagctgcgccgcctctggcttcacactgtctgactacggcatgcactgggtgagacaggctccagagaaaggactcgagtggttcgcctacatcagctctggcgactccacaatttactacgccgacaccgtgaagggaagattcaccatctctagggataacgccaagaacaccctgtttctgcagatgaccagcctgagatctgaggacacagctatgtactactgcgccagaagaatggcctactggggccagggcaccctggtcaccgtgtctgcc

SEQ ID NO: 92 - DNA, 1347 bp, Mus musculus
gaggtgcaactggttgagagcggcggtggcctggtgaaacctggcggctctctgaagctgagctgcgccgcctctggcttcacactgtctgactacggcatgcactgggtgagacaggctccagagaaaggactcgagtggttcgcctacatcagctctggcgactccacaatttactacgccgacaccgtgaagggaagattcaccatctctagggataacgccaagaacaccctgtttctgcagatgaccagcctgagatctgaggacacagctatgtactactgcgccagaagaatggcctactggggccagggcaccctggtcaccgtgtctgccgctaagaccacaccccctagcgtttatcctctggcccctggatgtggcgataccaccggatcaagcgtgacactgggctgcctagtgaagggctactttcctgagtctgtgaccgtgacatggaacagcggcagcctgtcttcttctgtacatacattcccagctcttctgcagagcggcctgtacaccatgagctccagcgtgacagtgcccagctccacatggcctagccagaccgtgacgtgtagcgtggcccaccccgccagcagcaccaccgtggacaagaagctggaacctagcggacctatcagcaccattaacccttgccccccctgcaaggaatgccacaagtgccctgcccctaatctggaaggcggccctagcgtgttcatcttcccccccaatatcaaggacgtgctgatgatctctctgacccctaaggttacatgcgtggtggtggatgtgtccgaggacgaccccgacgtgcggatctcctggttcgtgaacaacgtggaagtgcacaccgcgcagacacagacccaccgggaagattacaattctacaatccgggtggtctccgccctgccaatccagcaccaggactggatgagcggaaaggagttcaagtgtaaagttaacaacaaggacctgcctagtcctatcgagagaaccatcagcaagatcaagggcctggtgcgggcccctcaagtgtacatcctgccacctcctgccgagcagctgagcagaaaggatgtgagcctgacctgtctggtggtcggatttaaccccggcgacatctccgtggaatggaccagcaacggccacaccgaagagaactacaaggacaccgcccctgtgctggacagcgacggcagctacttcatctactccaaactcgatatcaaaaccagcaagtgggagaagactgatagcttcagctgcaacgtgcgccacgagggcctgaaaaattactatctgaagaagaccatcagcagaagccctggcaag

SEQ ID NO: 93 - DNA, 30 bp, Mus musculus
agcgcctctagcagcgtgacctacatgtac

SEQ ID NO: 94 - DNA, 21 bp, Mus musculus
gccaccagcaacctggcctct

SEQ ID NO: 95 - DNA, 27 bp, Mus musculus
cagcagtggtcctcctaccccctgacc

SEQ ID NO: 96 - DNA, 318 bp, Mus musculus
cagatcgtgctgacccagagccctgccatcatgtctgccagccctggcgagaaggtgacaatgacctgtagcgcctctagcagcgtgacctacatgtactggtaccagcaaaagcctggaagctctcccagactgctgatctacgccaccagcaacctggcctctggagtgcctgtgcggttcagcggctctggcagcggaactagctacagcctgaccatcagcagaatggaagctgaagatgccgccacctactactgccagcagtggtcctcctaccccctgaccttcggcgccggcacaaagctggaactgaag

SEQ ID NO: 97 - DNA, 639 bp, Mus musculus
cagatcgtgctgacccagagccctgccatcatgtctgccagccctggcgagaaggtgacaatgacctgtagcgcctctagcagcgtgacctacatgtactggtaccagcaaaagcctggaagctctcccagactgctgatctacgccaccagcaacctggcctctggagtgcctgtgcggttcagcggctctggcagcggaactagctacagcctgaccatcagcagaatggaagctgaagatgccgccacctactactgccagcagtggtcctcctaccccctgaccttcggcgccggcacaaagctggaactgaagcgggccgacgccgctcctacagtgtccatctttccacctagcagtgaacagctgacatctggcggcgccagcgtggtgtgcttcctgaacaacttctaccctaaggacattaacgtgaaatggaagatcgacggcagcgagcggcagaacggcgtgctgaatagctggaccgaccaagacagcaaggatagcacctattctatgtccagcaccctgacactgaccaaggacgagtacgagagacacaacagctatacatgcgaggctacccacaagacctccaccagccccatcgttaagagcttcaatagaaacgagtgc

SEQ ID NO: 98 - protein, 22 aa, Homo sapiens
GTPGSRSRTPSLPTPPTREPKK

SEQ ID NO: 99 - protein, 22 aa, Homo sapiens
  MISC_FEATURE (1)..(1): biotinylated
GTPGSRSRTPSLPTPPTREPKK

SEQ ID NO: 100 - protein, 22 aa, Homo sapiens
  MISC_FEATURE (1)..(1): biotinylated
  MISC_FEATURE (14)..(14): phosphorylated
GTPGSRSRTPSLPTPPTREPKK

SEQ ID NO: 101 - protein, 22 aa, Homo sapiens
  MISC_FEATURE (1)..(1): biotinylated
  MISC_FEATURE (9)..(9): phosphorylated
  MISC_FEATURE (11)..(11): phosphorylated
  MISC_FEATURE (17)..(17): phosphorylated
GTPGSRSRTPSLPTPPTREPKK

SEQ ID NO: 102 - protein, 22 aa, Homo sapiens
  MISC_FEATURE (1)..(1): biotinylated
  MISC_FEATURE (9)..(9): phosphorylated
  MISC_FEATURE (14)..(14): phosphorylated
GTPGSRSRTPSLPTPPTREPKK

SEQ ID NO: 103 - protein, 22 aa, Homo sapiens
  MISC_FEATURE (1)..(1): biotinylated
  MISC_FEATURE (11)..(11): phosphorylated
  MISC_FEATURE (14)..(14): phosphorylated
GTPGSRSRTPSLPTPPTREPKK

SEQ ID NO: 104 - protein, 22 aa, Homo sapiens
  MISC_FEATURE (1)..(1): biotinylated
  MISC_FEATURE (14)..(14): phosphorylated
  MISC_FEATURE (17)..(17): phosphorylated
GTPGSRSRTPSLPTPPTREPKK

SEQ ID NO: 105 - protein, 22 aa, Homo sapiens
  MISC_FEATURE (1)..(1): biotinylated
  MISC_FEATURE (9)..(9): phosphorylated
  MISC_FEATURE (11)..(11): phosphorylated
  MISC_FEATURE (14)..(14): phosphorylated
GTPGSRSRTPSLPTPPTREPKK